(19) United States
(12) Patent Application Publication
(10) Pub. No.: US 2016/0250266 A1
(43) Pub. Date: Sep. 1, 2016

(54) PURE FILAMENTOUS BACTERIOPHAGE AND METHODS OF PRODUCING SAME

(71) Applicant: NeuroPhage Pharmaceuticals, Inc., Cambridge, MA (US)

(72) Inventors: Jason Wright, San Francisco, CA (US); Antony Hitchcock, Crewe (GB); Frank Sugar, Athens, GA (US); Tim Davies, Ceredigion (GB); Shreekant Adhikari, Athens, GA (US); Nanda Menon, Watkinsville, GA (US); Quentin Florence, Loganville, GA (US)

(21) Appl. No.: 14/964,858

(22) Filed: Dec. 10, 2015

Related U.S. Application Data
(63) Continuation of application No. 13/566,274, filed on Aug. 3, 2012, now abandoned.
(60) Provisional application No. 61/515,726, filed on Aug. 5, 2011.

(51) Int. Cl. A61K 35/76; C12N 7/00

(52) U.S. Cl. CPC A61K 35/76; C12N 7/00; C12N 2795/00032; A61K 2035/11

(57) ABSTRACT

The invention relates to compositions of purified filamentous bacteriophage, as well as methods that allow reproducible purification of high concentrations of filamentous bacteriophage.

This application claims the benefit of priority of U.S. Provisional Patent Application No. 61/515,726, filed Aug. 5, 2011, which is incorporated by reference in its entirety herein.

The invention relates to compositions of filamentous bacteriophage having sufficiently low levels of host cell contaminants, such as bacterial endotoxin, for use in the preparation of therapeutically effective pharmaceutical compositions, as well as drug product and pharmaceutical compositions prepared therefrom. The invention also relates to methods for producing such compositions.

Filamentous bacteriophage are emerging as therapeutic agents for treatment of neurodegenerative diseases and disorders, including Parkinson's disease or susceptibility to Parkinson's disease (see PCT Patent Publication WO20100060073), and diseases and disorders characterized by amyloid plaque formation in the brain and elsewhere in the body (see, e.g., U.S. Patent Publication 20110142803, U.S. Patent Publication 20090180991, and PCT patent publication WO2008011503). Filamentous bacteriophage are also emerging as therapeutic agents for treatment of neurodegenerative tauopathies (see PCT Patent Application No. PCT/US2012/028762, filed Mar. 12, 2012). These references also indicate that filamentous bacteriophage can reduce susceptibility to neurodegenerative tauopathies and/or plaque forming diseases. In addition, filamentous bacteriophage engineered to express a therapeutic agent, antigen, or antibody have also been suggested as useful therapeutic agents. See, for example, PCT patent publications WO2002074243, WO2004030694, WO2007094003, and WO2007001302; and U.S. Patent Publication US20020044922.

Filamentous bacteriophage are produced by fermentation, using gram-negative bacterial cell hosts for their growth. Gram-negative bacteria are cultured with a complex growth medium, containing sugars, amino acids, and growth factors, usually supplied from preparations of animal serum. Bacterial DNA and proteins are undesirable contaminants that are typically found in the fermentation media along with the phage. Moreover, gram-negative bacteria produce endotoxin, a toxic and highly undesirable contaminant in any therapeutic agent, which is difficult to separate from the filamentous bacteriophage. The United States Food and Drug Administration has set forth guidelines for the maximum amount of endotoxin allowed in drug products at 5.0 endotoxin units (“EU”)/kg body weight/dose and at 0.2 EU/kg/dose for intrathecally injected drug products. See Food and Drug Administration Inspection Technical Guide No. 40, Mar. 20, 1985, available as file ucm07298.htm in the ICECI/Inspections/InspectionGuides/InspectionTechnicalGuides subdirectory of the FDA website (URL: http://www.fda.gov/ICECI/Inspections/InspectionGuides/InspectionTechnicalGuides/ucm072918.htm). Accordingly, the difficulties associated with large-scale, economic purification of filamentous bacteriophage are an increasingly important problem for the biotechnology industry.

Advances in fermentation techniques have greatly increased the concentration of filamentous bacteriophage capable of being produced in any given composition. This increase in upstream efficiency has led, however, to difficulties in downstream processing. Producing higher concentrations of bacteriophage requires higher concentrations of bacterial hosts and concomitantly higher concentrations of bacterial DNA, proteins and endotoxin. Bacteriophage must be separated from the bacterial hosts in which they grow and these bacterial by-products present in the fermentation media in order to be used as therapeutic compositions.

Procedures for purification of filamentous bacteriophage have typically relied on PEG precipitation and CsCl gradients formed by ultracentrifugation. See, for example, Sambrook J. and Russell D. W. “Molecular Cloning. A Laboratory Manual”; Third Edition (2001) at Chapter 3. The bacteriophage produced by these procedures are not adequate for therapeutic use because the procedures do not remove sufficient quantities of bacterial cell by-products to allow for administration to humans. Thus, improved methods for purifying compositions of filamentous bacteriophage are greatly needed.

The purification techniques must be scaleable, efficient, cost-effective, reliable, and meet the rigorous purity requirements of the final product.

The present invention is based in part on the discovery of novel purification techniques resulting in filamentous bacteriophage compositions comprising acceptably low levels of bacterial cell contaminants, such as, for example, endotoxin. These novel purification techniques are scaleable, efficient, cost-effective and reliable. Most importantly, however, the purification techniques of this invention are useful to produce filamentous bacteriophage compositions that are suitable for administration to humans. The levels of endotoxin are low enough to allow for any type of administration, including, for example, direct injection into the brain, which may be the preferred delivery method in many diseases characterized by plaque formation in the brain.

Methods for purifying high concentrations of filamentous bacteriophage on a large scale are vital for the commercial preparation of therapeutic filamentous bacteriophage to be used in the treatment and prevention of neuronal diseases and disorders.

Embodiments of the invention include compositions comprising filamentous bacteriophage having an endotoxin to phage ratio of less than 5×10⁻¹⁴ endotoxin units (“EU”) per phage. The compositions may also comprise filamentous bacteriophage having an endotoxin to phage ratio of less than 1×10⁻¹³ EU per phage, less than 1×10⁻¹² EU per phage, less than 1×10⁻¹¹ EU per phage, and less than 1×10⁻¹⁰ EU per phage.

Further embodiments of the invention include compositions comprising wild-type filamentous bacteriophage or filamentous phage which does not display an antibody or a non-filamentous bacteriophage antigen on its surface, said composition comprising less than 1×10⁻¹⁰ endotoxin units per filamentous bacteriophage, less than 1×10⁻¹¹ EU per phage, less than 1×10⁻¹² EU per phage, less than 1×10⁻¹³ EU per phage, or less than 5×10⁻¹⁴ EU per phage.

Additional embodiments of the invention include compositions comprising filamentous bacteriophage for use in the diagnosis, treatment or prevention of a brain disease or a disease characterized by the presence of amyloid plaque, said composition comprising less than 1×10⁻¹⁰ endotoxin units per filamentous bacteriophage, less than 1×10⁻¹¹ EU per phage, less than 1×10⁻¹² EU per phage, less than 1×10⁻¹³ EU per phage, or less than 5×10⁻¹⁴ EU per phage. In still further embodiments, the invention provides methods for the diagnosis, treatment or prevention of a brain disease or a disease characterized by the presence of amyloid plaque, comprising administering to a subject in need thereof a composition comprising less than 1×10⁻¹⁰ endotoxin units per filamentous bacteriophage, less than 1×10⁻¹¹ EU per phage, less than 1×10⁻¹² EU per phage, less than 1×10⁻¹³ EU per phage, or less than 5×10⁻¹⁴ EU per phage.

### BRIEF DESCRIPTION OF THE DRAWINGS

FIG. 1 is a chromatogram from the Phenyl HIC step. Fluorescence emission at 334 nm is measured after excitation at 242 nm. The M13 peak is labeled. In this example, 420 mLs of the M13 containing retentate from the first ultrafiltration step was diluted with equal volume of 25 mM Tris pH 7.4/4M NaCl, and loaded onto the Phenyl HIC column with a peristaltic pump at 100 mL/min. M13 was eluted with a step gradient of 25 mM Tris, pH 7.4, 250 mM NaCl after a wash step with 25 mM Tris, pH 7.4, 2M NaCl.

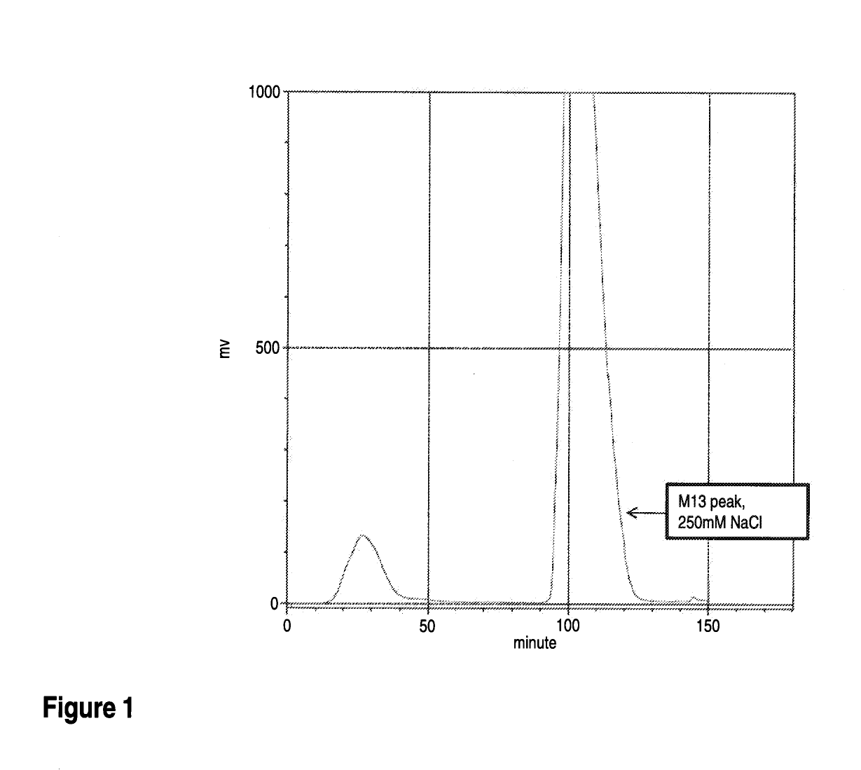

FIG. 2 is a chromatogram from the Phenyl HIC step. Fluorescence is shown (Ex. 242 nm; Em 334 nm). The M13 peak is labeled. In this example, 320 mLs of the M13 containing retentate from the first ultrafiltration step was diluted with an equal volume of 25 mM Tris pH 7.4/4M NaCl and loaded onto a Phenyl HIC column. M13 was eluted with a step gradient of 25 mM Tris, pH 7.4, 250 mM NaCl, after a wash step with 25 mM Tris, pH 7.4, 2M NaCl.

FIG. 3 is a chromatogram from the Phenyl HIC step. Absorbance at A254 nm is shown. The M13 peak is labeled. In this example, 320 mLs of the M13 containing retentate from the first ultrafiltration step was diluted with equal volume of 25 mM Tris pH 7.4/4M NaCl and loaded onto a Phenyl HIC column. M13 was eluted with a step gradient of 25 mM Tris, pH 7.4, 250 mM NaCl, after a wash step with 25 mM Tris, pH 7.4, 2M NaCl

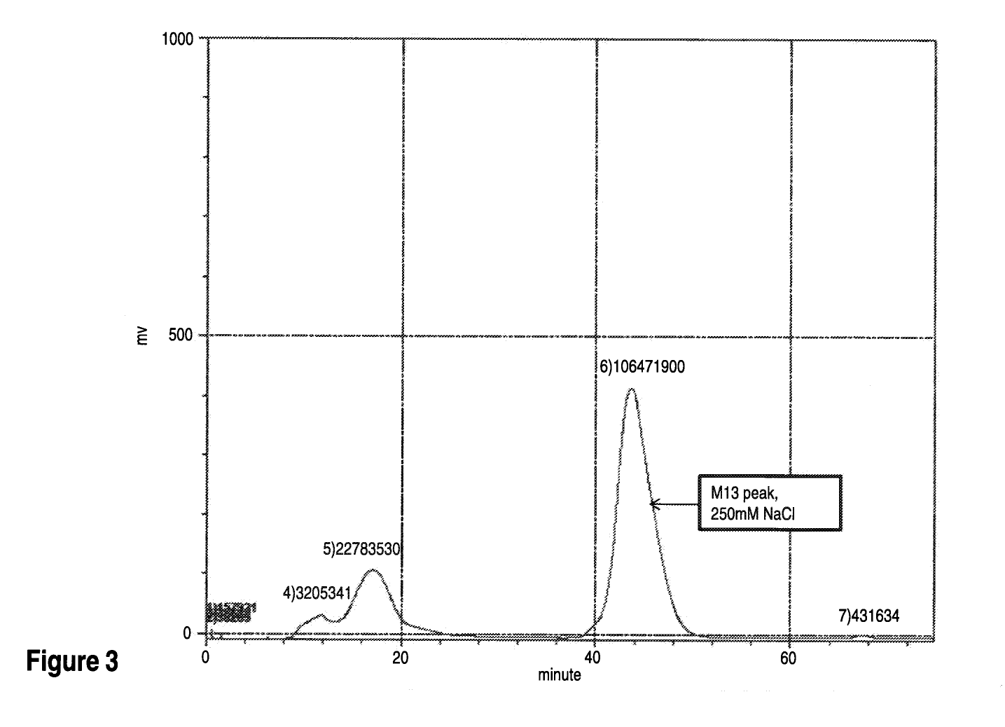

FIG. 4 is a chromatogram from the DEAE AEX step. Fluorescence is shown (Ex. 242 nm; Em 334 nm). The M13 peak is labeled. In this example, eluate from the HIC Phenyl step, which contains M13, was diluted six times with 25 mM Phosphate, pH 6.5 and loaded onto the DEAE column with a peristalitic pump at 100 ml/min. M13 was eluted with a step gradient in 25 mM Phosphate, pH 6.5, 300 mM NaCl after successive washes with 25 mM Phosphate, pH 7.4, 150 mM NaCl and 25 mM Phosphate, pH 7.4, 250 mM NaCl at a flow rate of 100 ml/min.

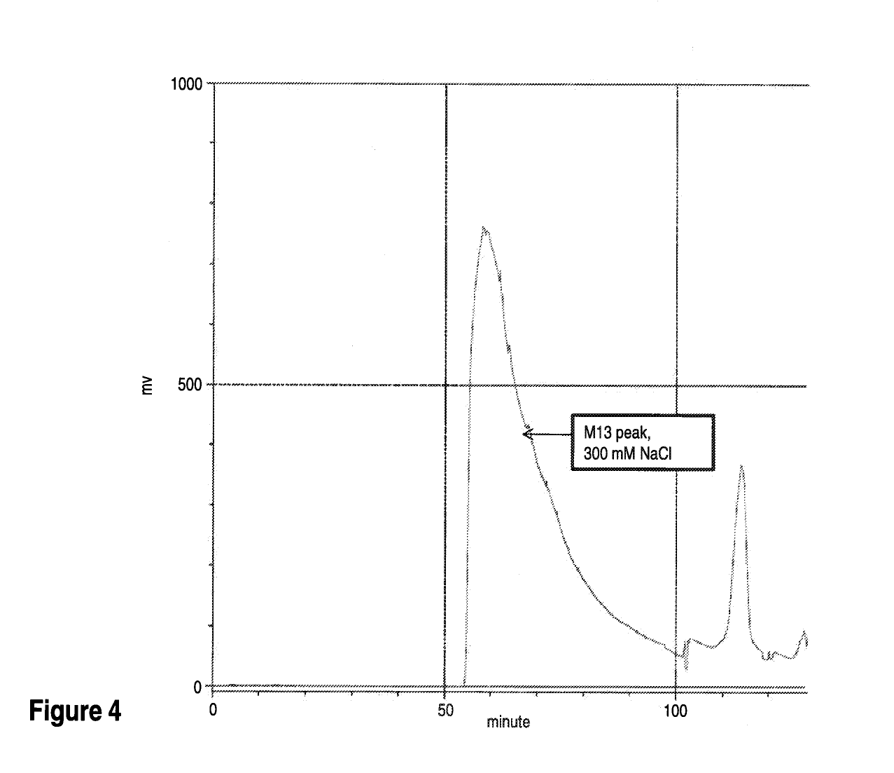

FIG. 5 is a chromatogram from the DEAE AEX step. Fluorescence at excitation at 242 nm and Emission at 334 nm is shown. The M13 peak is labeled. In this example, approximately 2 L of the eluate from the HIC Phenyl step, which contains M13, was diluted with 10 L of 25 mM Phosphate pH 6.5 and loaded onto the DEAE column. M13 was eluted with a step gradient of 25 mM Phosphate, pH 6.5, 300 mM NaCl after successive washes with 25 mM Phosphate, pH 7.4, 150 mM NaCl and 25 mM Phosphate, pH 7.4, 250 mM NaCl.

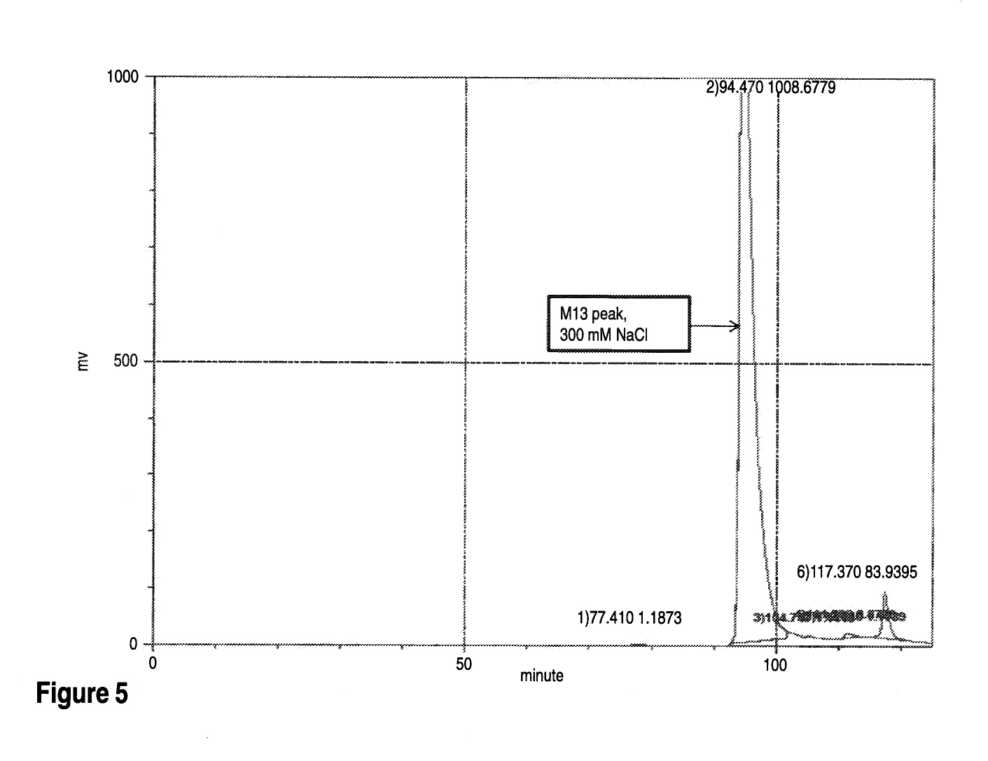

FIG. 6 is a chromatogram from the AEX Q step. Fluorescence at excitation 242 nm and Emission at 334 nm is shown (the corresponding adsorbance trace for this run is provided in FIG. 7). The M13 peak is labeled. In this example, approximately 750 mL of the eluate from the DEAE AEX step, which contains M13, was diluted with 750 mL of 25 mM Tris pH 7.4 and loaded onto the AEX Q column. M13 was eluted with a step gradient of 25 mM Tris, pH 7.4, 280 mM NaCl after a wash step with 25 mM Tris, pH 7.4, 200 mM NaCl.

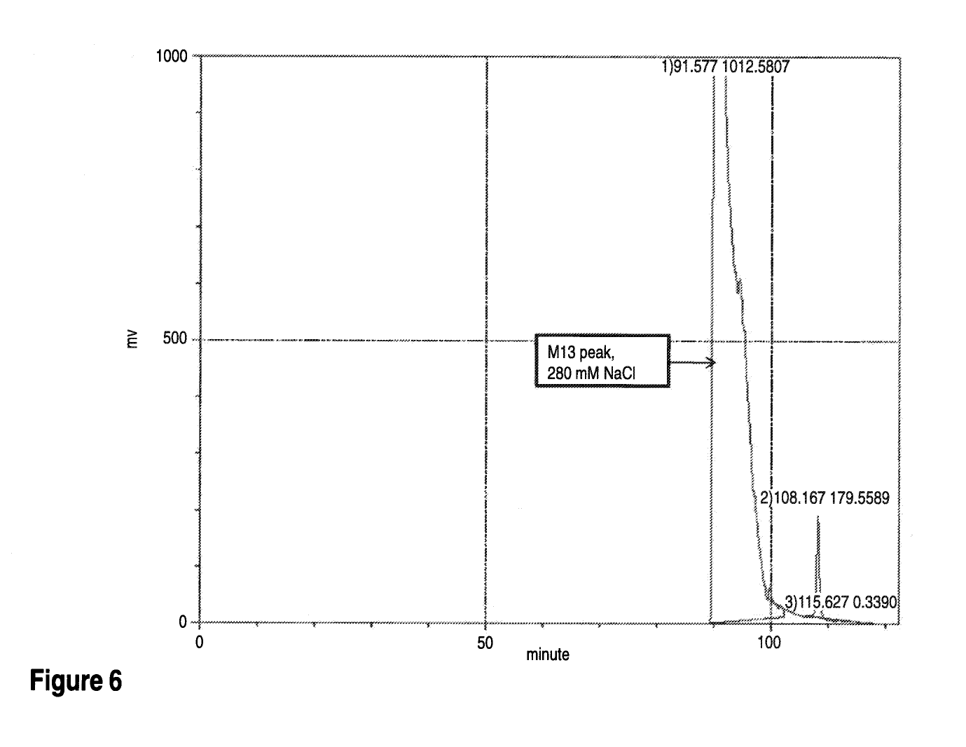

FIG. 7 is a chromatogram from the AEX Q step. Absorbance at A254 nm is shown (the corresponding fluorescence trace for this run is provided in FIG. 6). The M13 peak is labeled In this example, approximately 750 mL of the eluate from the DEAE AEX step, which contains M13, was diluted with 750 mL of 25 mM Tris pH 7.4 and loaded onto the AEX Q column. M13 was eluted with a step gradient of 25 mM Tris, pH 7.4, 280 mM NaCl after a wash step with 25 mM Tris, pH 7.4, 200 mM NaCl.

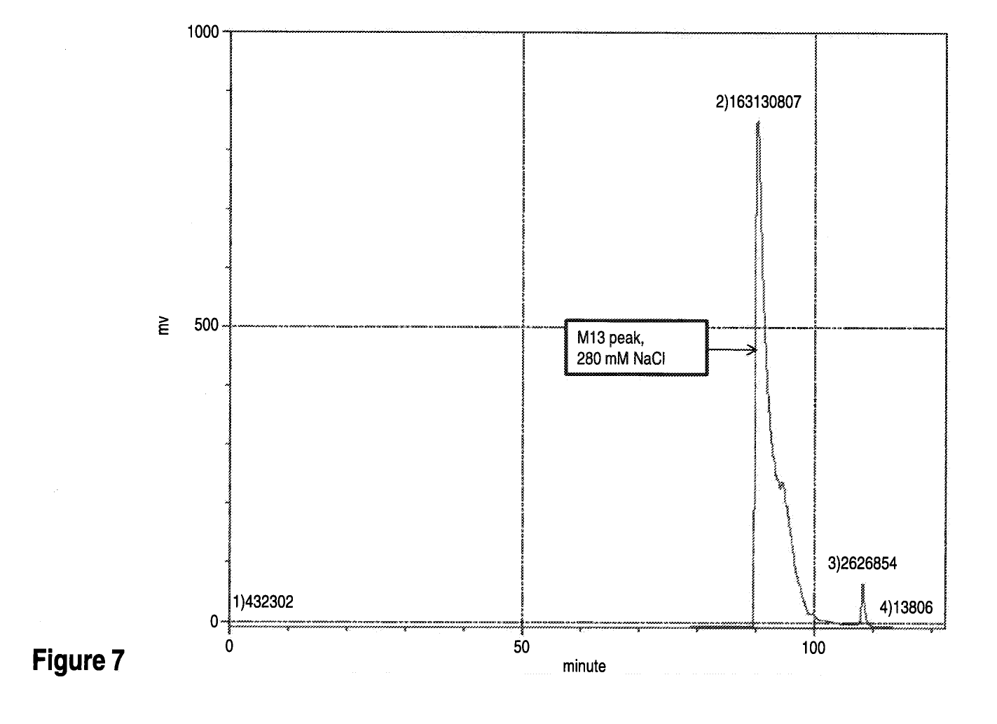

FIG. 8 is a chromatogram from the Phenyl HIC step. Fluorescence at excitation 242 nm and Emission at 334 nm is shown. The M13 peak is labeled. In this example, 400 mLs of the M13 containing retentate from the first ultrafiltration step was diluted with equal volume of 25 mM Tris pH 7.4/4M NaCl, and loaded onto the Phenyl HIC column with a peristaltic pump at 100 mL/min. M13 was eluted with a step gradient of 25 mM Tris, pH 7.4, 250 mM NaCl after a wash step with 25 mM Tris, pH 7.4, 2M NaCl.

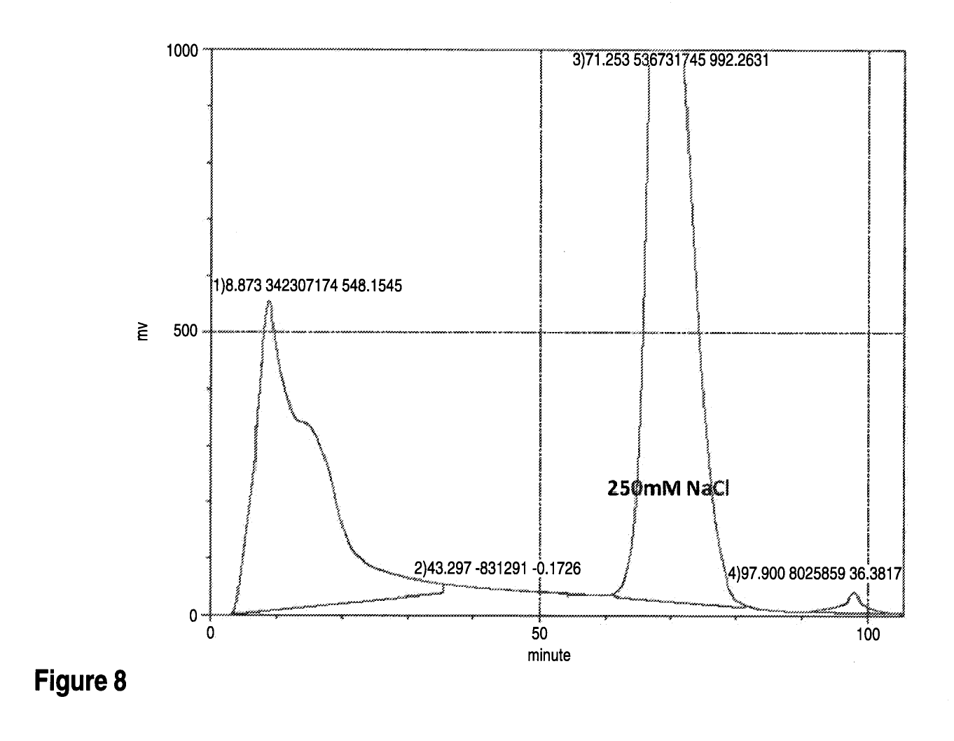

FIG. 9 is a chromatogram from the DEAE step. Fluorescence at excitation 242 nm and Emission at 334 nm is shown. The M13 peak is labeled. In this example, eluate from the HIC Phenyl step, which contains M13, was diluted six times with 25 mM Phosphate, pH 6.5 and loaded onto the DEAE column with a peristalitic pump at 100 ml/min. M13 was eluted with a step gradientof 25 mM Phosphate, pH 6.5, 300 mM NaCl after successive washes with 25 mM Phosphate, pH 7.4, 150 mM NaCl and 25 mM Phosphate, pH 7.4, 250 mM NaCl.

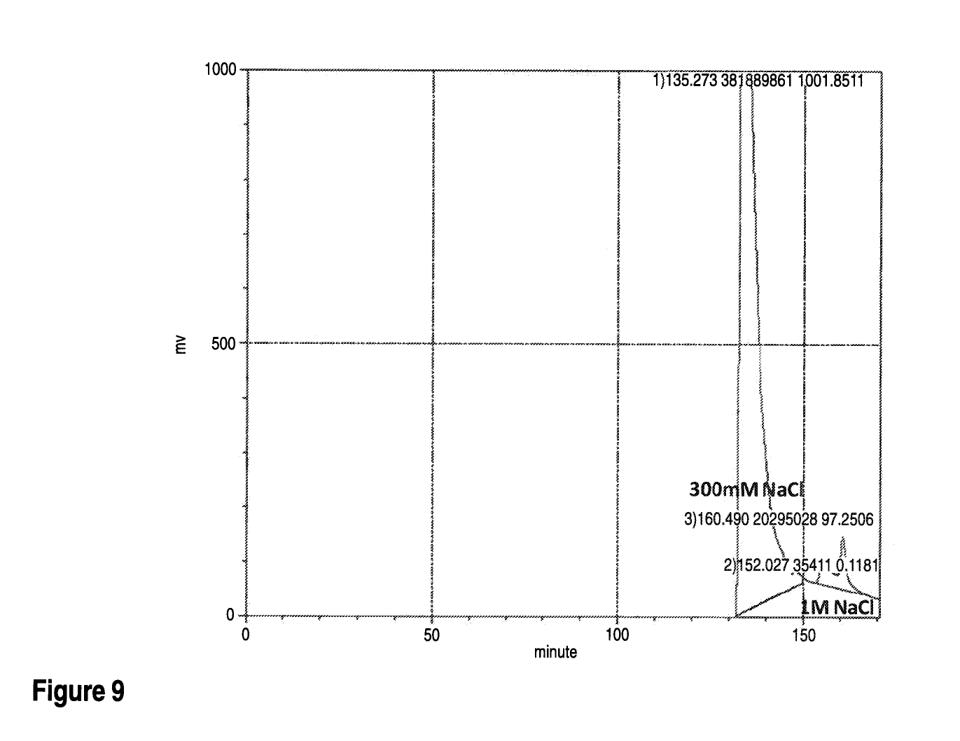

FIG. 10 is a chromatogram from the AEX Q step. Fluorescence at excitation 242 nm and Emission at 334 nm is shown. The M13 peak is labeled. In this example, the eluate from the DEAE AEX step, which contains M13, was diluted with an equal volume of 25 mM Tris pH 7.4 and loaded onto the AEX Q column. M13 was eluted with a step gradient of 25 mM Tris, pH 7.4, 280 mM NaCl after a wash step with 25 mM Tris, pH 7.4, 200 mM NaCl.

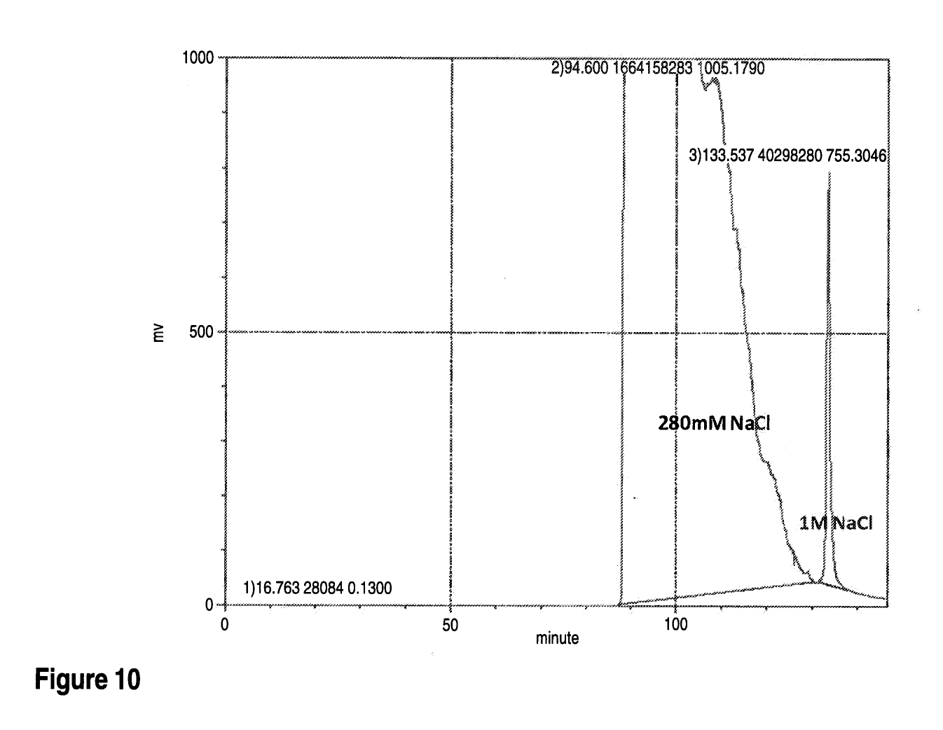

FIG. 11 is a chromatogram from the Phenyl HIC step. Fluorescence at excitation 242 nm and Emission at 334 nm is shown. The M13 peak is labeled. In this example, the supernatant from the depth filtration step, which contains M13, was diluted with equal volume of 25 mM Tris pH 7.5/4M NaCl, and loaded onto the Phenyl HIC column with a peristaltic pump at 100 mL/min. M13 was eluted with a step gradient of 25 mM Tris, pH 7.4, 250 mM NaCl, after a wash step with 25 mM Tris, pH 7.4, 2M NaCl.

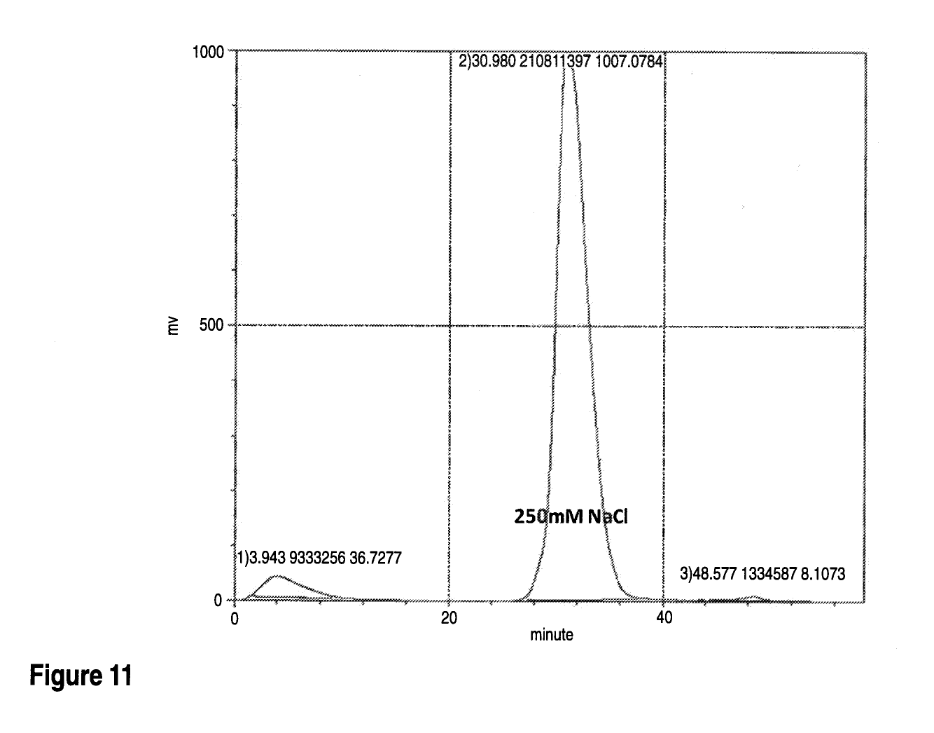

FIG. 12 is a chromatogram from the DEAE step. Fluorescence at excitation 242 nm and Emission at 334 nm is shown. The M13 peak is labeled. In this example, 3 L of the eluate from the HIC Phenyl step, which contains M13, was diluted with 10 L 25 mM Phosphate, pH 6.5 and loaded onto the DEAE column with a peristalitic pump at 100 ml/min. M13 was eluted with a step gradient of 25 mM Tris, pH 7.4, 300 mM NaCl after a wash step with 25 mM Tris, pH 7.4, 200 mM NaCl.

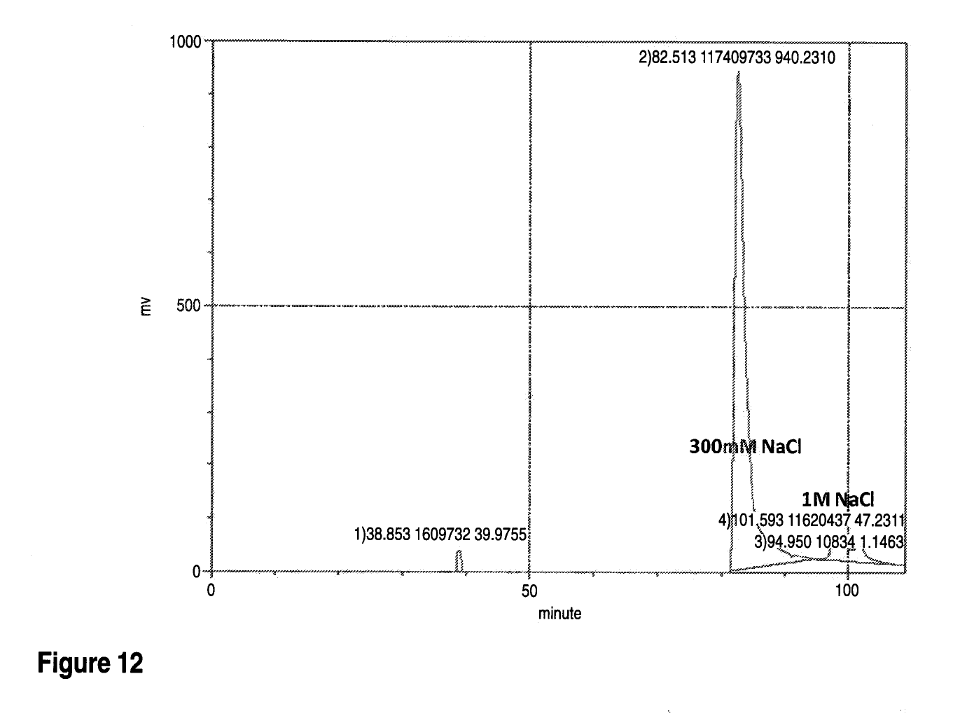

FIG. 13 is a chromatogram from the AEX Q step. Fluorescence at excitation 242 nm and Emission at 334 nm is shown. The M13 peak is labeled. In this example, approximately 3 L of the eluate from the DEAE AEX step, which contains M13, was diluted with 2 L of 25 mM Tris pH 7.4 and loaded onto the AEX Q column. M13 was eluted with a step gradient of 25 mM Tris, pH 7.4, 280 mM NaCl after a wash step with 25 mM Tris, pH 7.4, 200 mM NaCl.

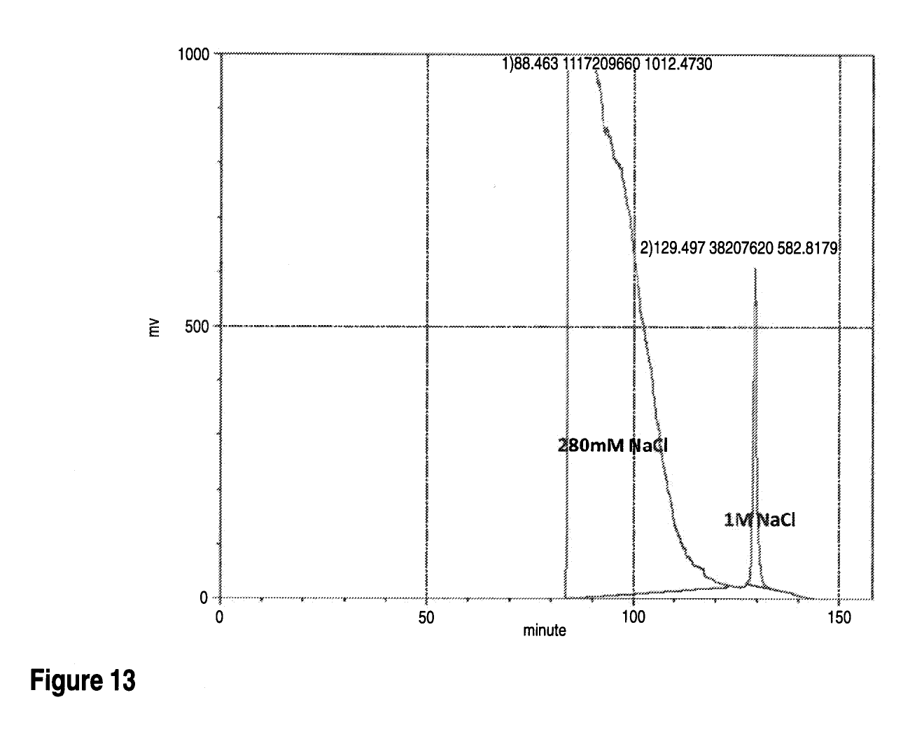

FIG. 14 shows the elution profile of M13 purified with the process described in Example 5 from an analytical AEX column (ProSwift WAX-1S), 5 μl of neat M13 was diluted with 75 μl of Buffer A (50 mM Phosphate, pH 7.5). M13 was eluted in a linear gradient from 100% Buffer A to 100% Buffer B (50 mM Phosphate, pH 2.2/2M NaCl).

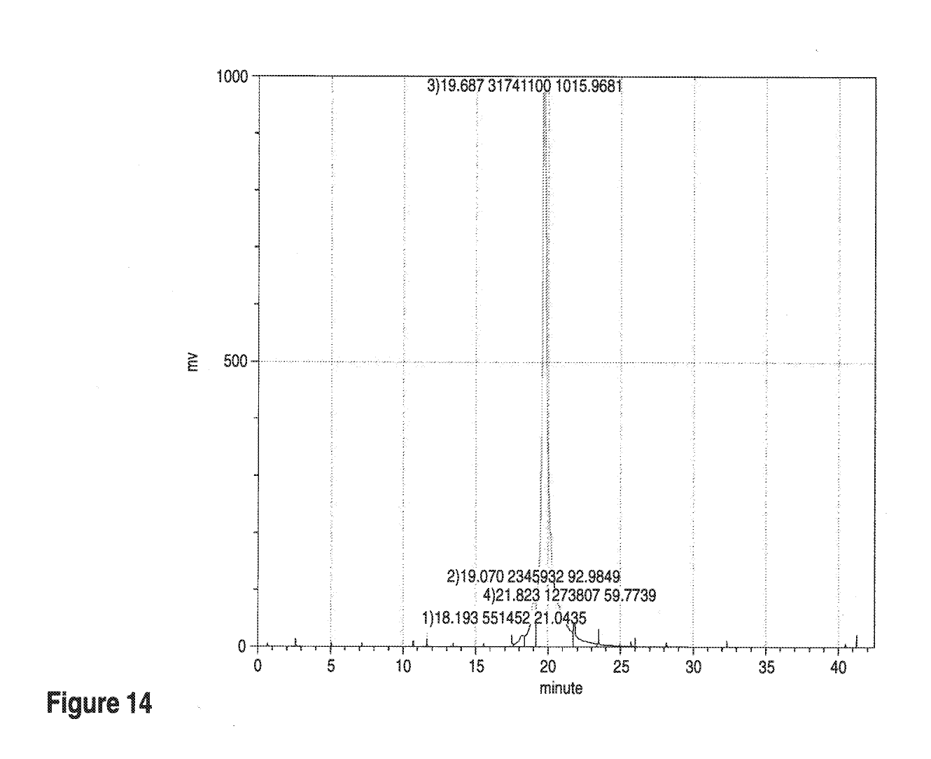

FIG. 15 shows an image of an SDS PAGE Gel stained with Coomassie, where column 1 is loaded with the filamentous bacteriophage produced by the purification procedure outlined in Example 5. Column 2 is loaded with 10 μl of a molecular weight marker (Marker 12; Invitrogen), and column 3 with a positive control (reference M13; Batch 5). M13 is loaded at 1.5×10¹¹ in all lanes (except marker). This gel shows the presence of the major coat protein g8p and the lack of other major protein contaminant bands.

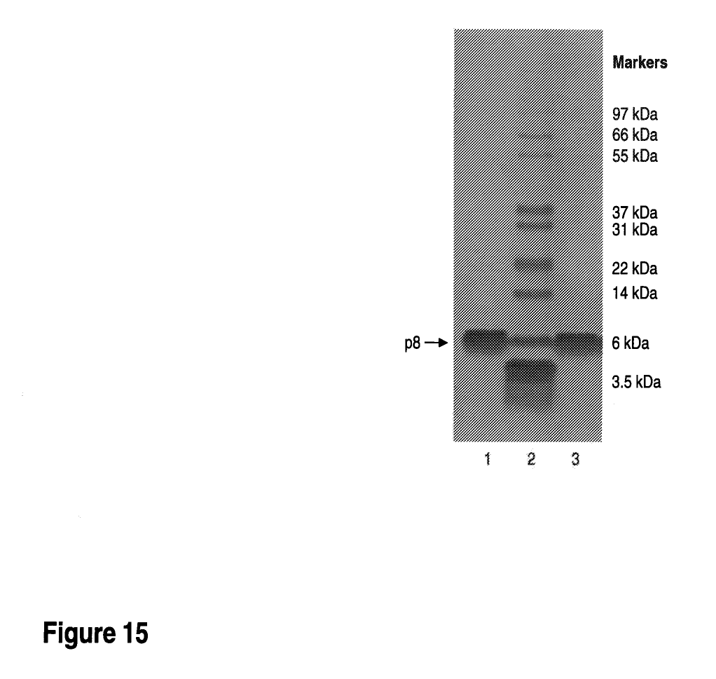

FIG. 16 shows the elution profile of M13 purified with the process described in Example 4 (Batch 2) from an analytical AEX column (ProSwift WAX-1S). 5 μl of neat M13 was diluted with 75 μl of Buffer A (50 mM Phosphate, pH 7.5). M13 was eluted in a linear gradient from 100% Buffer A to 100% Buffer B (50 mM Phosphate, pH 2.2/2M NaCl).

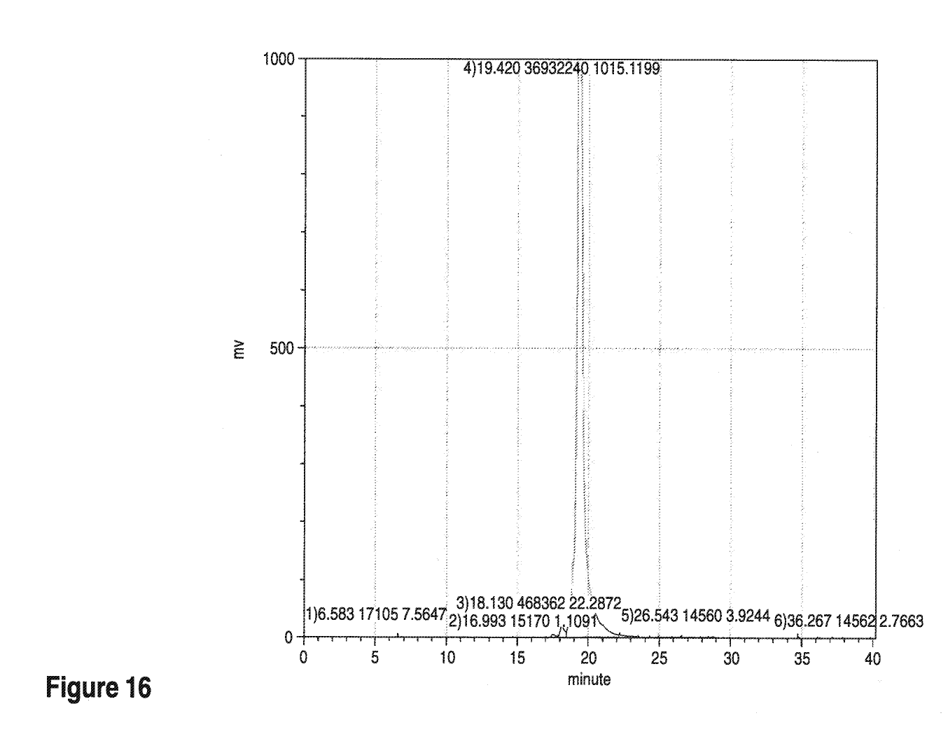

FIG. 17 shows an SDS PAGE Gel, where column 1 is loaded with a positive control (Batch 5), column 2 is loaded with the filamentous bacteriophage produced by the purification procedure outlined in Example 4 (Batch 2), and column 3 with 10 μl of a molecular weight marker (Marker 12; Invitrogen). M13 is loaded at 1.5×10¹¹ in all lanes (except marker). This gel shows the presence of the major coat protein g8p and the lack of other major protein contaminant bands.

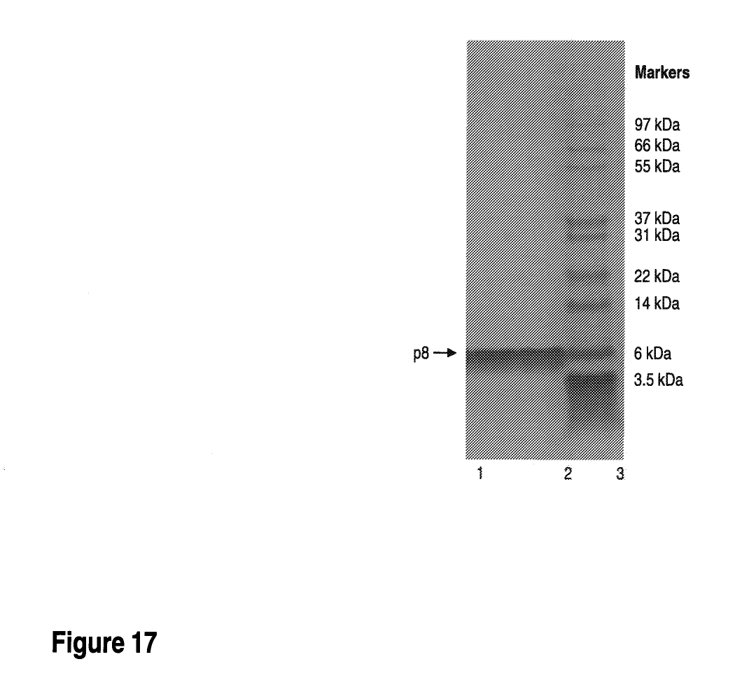

FIG. 18 shows the elution profile of M13 produced with the PEG precipitation and 2×CsCl density gradient (ultracentrifugation method) from an analytical AEX column (ProSwift WAX-1S). See, Example 7. 5 μl of neat M13 was diluted with 75 μl of Buffer A (50 mM Phosphate, pH 7.5). M13 was eluted in a linear gradient from 100% Buffer A to 100% Buffer B (50 mM Phosphate, pH 2.2/2M NaCl).

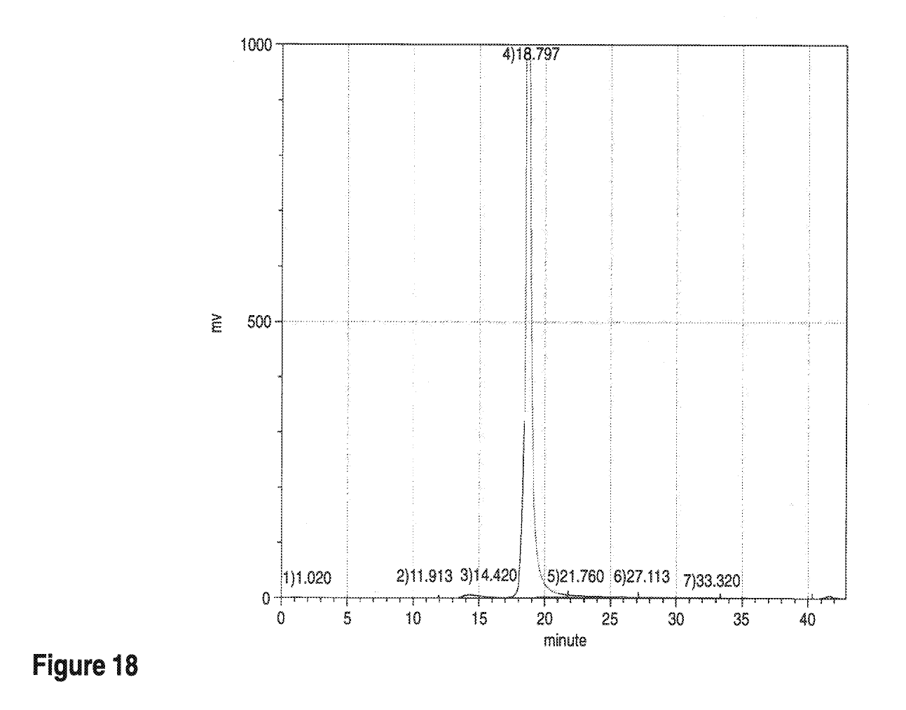

FIG. 19 shows an SDS PAGE Gel, where column 1 is loaded with an M13 batch generated using the PEG precipitation and 2×CsCl density gradient method. See, Example 7. Column 2 is loaded with 10 μl of a molecular weight marker (Marker 12; Invitrogen), and column 3 is loaded with a positive control (Batch 2; Example 4) sample of purified filamentous bacteriophage (Batch 2; Example 4), and column 3 with a marker. M13 is loaded at 1.5×10¹¹ in all lanes (except marker). This gel shows the presence of the major coat protein g8p and the lack of other major protein contaminant bands.

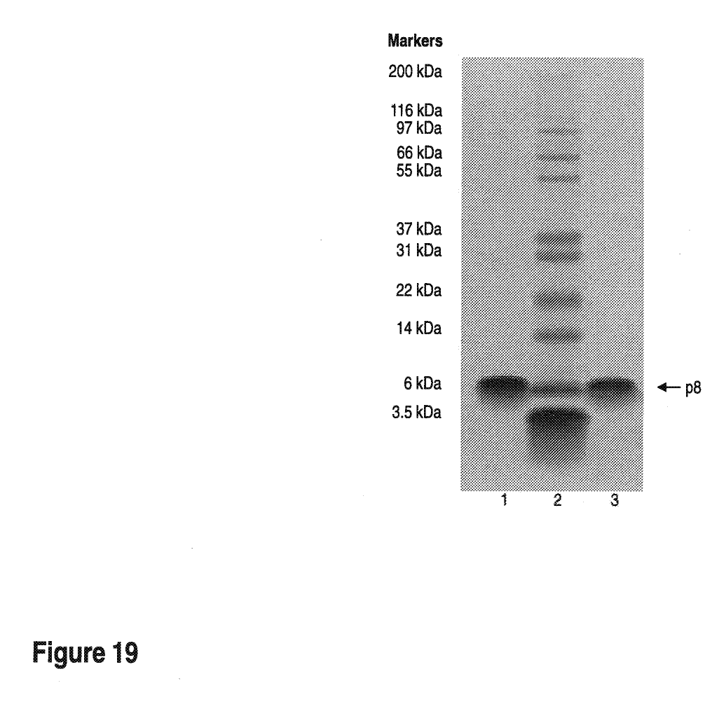

### DESCRIPTION OF EMBODIMENTS

Definitions

Filamentous bacteriophage are a group of related viruses that infect gram negative bacteria, such as, e.g., E. coli. See, e.g., Rasched and Oberer, Microbiology Reviews (1986) December: 401-427. In the present application, filamentous bacteriophage may also be referred to as “bacteriophage,” or “phage.” Unless otherwise specified, the term “filamentous bacteriophage” includes both wild type filamentous bacteriophage and recombinant filamentous bacteriophage.

“Wild type filamentous bacteriophage” refers to filamentous bacteriophage that express only filamentous phage proteins and do not contain any heterologous nucleic acid sequences, e.g. non-phage sequences that have been added to the bacteriophage through genetic engineering or manipulation. One such wild-type filamentous bacteriophage useful in the invention is M13. The term “M13” is used herein to denote a form of M13 phage that only expresses M13 proteins and does not contain any heterologous nucleic acid sequences. M13 proteins include those encoded by M13 genes I, II, III, IIIp, IV, V, VI, VII, VIII, VIIIp, IX and X. van Wezenbeek et al. Gene (1980) 11:129-148.

Suitable wild type filamentous bacteriophage for use in the compositions and methods of the invention include at least M13, f1, or fd, or mixtures thereof. Although M13 was used in the Examples presented below, any closely related wild type filamentous bacteriophage is expected to behave and function similarly to M13. Closely related wild type filamentous bacteriophage refer to bacteriophage that share at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity, to the sequence of M13, f1, or fd at the nucleotide or amino acid level. In some embodiments, closely related filamentous bacteriophage refers to bacteriophage that share at least 95% identity to the DNA sequence of M13 (See, e.g., GenBank:V00604; Refseq: NC 003287).

“Recombinant filamentous bacteriophage” refers to filamentous bacteriophage that have been genetically engineered to express at least one non-filamentous phage protein and/or comprise at least one heterologous nucleic acid sequence. For example, recombinant filamentous bacteriophage may be engineered to express a therapeutic protein, including, e.g., an antibody, an antigen, a detectable marker (for diagnostic use), a peptide that modulates a receptor, a peptide composed of beta-breaker amino acids like proline, cyclic peptides made of alternating D and L residues that form nanotubes, and a metal binding protein.

The filamentous bacteriophage compositions of the invention may be purified in any desired volume by adjusting the processes set forth below as necessary and as would be readily understood by those of skill in the art. In each embodiment, the compositions comprise filamentous bacteriophage or recombinant filamentous bacteriophage that have been purified to reduce the levels of bacterial cell contaminants, such as, for example, endotoxin. The levels of endotoxin are sufficiently low to administer to humans via any route of administration, including, for example, direct injection into the brain. In one embodiment, the purified filamentous bacteriophage have a concentration of at least 4×10¹² phage/ml, at least 1×10¹³ phage/ml, at least 5×10¹³ phage/ml, at least 9×10¹³ phage/ml, or at least 1×10¹⁴ phage/ml. Importantly, the EU/phage ratio is less than 1×10⁻¹⁰ EU/phage, less than 1×10⁻¹¹ EU/phage, less than 1×10⁻¹² EU/phage, less than 1×10⁻¹³ EU/phage, or less than 5×10⁻¹⁴ EU/phage.

“Endotoxin” is found in the outer cell membrane of all gram-negative bacteria. “Endotoxin” may also be referred to as “lipopolysaccharide” or “LPS” throughout.

As used herein a “pharmaceutical composition” refers to a preparation of filamentous bacteriophage described herein with other chemical components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered filamentous bacteriophage compound. An adjuvant is included under these phrases.

The term “excipient” refers to an inert substance added to a pharmaceutical composition to further facilitate administration of an active ingredient. Examples, without limitation, include, for example, calcium carbonate, calcium phosphate, various sugars and types of starch, cellulose derivatives, gelatin, vegetable oils, polyethylene glycols, and surfactants, including, for example, polysorbate 20.

The term “dose” refers to an amount administered to a patient, particularly a human, over not more than one hour. “Dose” includes single bolus or solid dosage forms, as well as infusions and amounts delivered by implanted pumps.

The term “unit dosage form” or “single dosage form” generally refers to the drug product of the invention that is intended to provide delivery of a single dose of a drug to the patient at the time of administration for use, e.g., in homes, hospitals, facilities, etc. The drug product is dispensed in a unit dose container—a non-reusable container, tablet, pill, etc. designed to hold a quantity of drug intended for administration (other than the parenteral route) as a single dose, directly from the container, tablet, pill, etc., employed generally in a unit dose system. The advantages of unit dose dispensing are that the drug is fully identifiable and the integrity of the dosage form is protected until the actual moment of administration. If the drug is not used and the container, tablet, pill, etc. is intact, the drug may be retrieved and redispensed without compromising its integrity.

The term “retentate” refers to the part of a solution that does not cross a filtration membrane. This is in contrast to the “permeate” part of the solution that passes across the membrane.

As used herein, the term “eluate” generally refers to an entity that is released from another entity by a changing solvent condition (e.g. the release of bound M13 from a charged chromatography matrix by increasing the salt concentration).

The term “treating” is intended to mean substantially inhibiting, slowing or reversing the progression of a disease, substantially ameliorating clinical symptoms of a disease or substantially preventing the appearance of clinical symptoms of a disease. Also as used herein, the term “plaque forming disease” refers to diseases characterized by formation of plaques by an aggregating protein (plaque forming peptide), such as, but not limited to, alpha-synuclein, beta-amyloid, serum amyloid A, cystatin C, IgG kappa light chain, tau protein, or prion protein. Such diseases include, but are not limited to, early onset Alzheimer's disease, late onset Alzheimer's disease, presymptomatic Alzheimer's disease, SAA amyloidosis, hereditary Icelandic syndrome, senility, multiple myeloma, to prion diseases that are known to affect humans (such as for example, kuru, Creutzfeldt-Jakob disease (CJD), Gerstmann-Straussler-Scheinker disease (GSS), and fatal familial insomnia (FFI)) or animals (such as, for example, scrapie and bovine spongiform encephalitis (BSE)), Parkinson's Disease, Argyrophilic grain dementia, Corticobasal degeneration, Dementia pugilistica, diffuse neurofibrillary tangles with calcification, Down's syndrome, Frontotemporal dementia with parkinsonism linked to chromosome 17, Hallervorden-Spatz disease, Myotonic dystrophy, Niemann-Pick disease type C, Non-Guamanian motor neuron disease with neurofibrillary tangles, Pick's disease, Postencephalitic parkinsonism, Progressive subcortical gliosis, Progressive supranuclear palsy, Subacute sclerosing panencephalitis, and Tangle only dementia.

Compositions

In some embodiments, the invention provides large-scale compositions of filamentous bacteriophage. The term “large-scale composition” refers to a composition that comprises a sufficient number of filamentous bacteriophage for at least 10, 100, 1,000, 10,000, 100,000, or more therapeutically effective doses. In some aspects of this embodiment, the compositions comprise at least 2×10¹⁶ to 4.5×10²¹ total filamentous bacteriophage. The filamentous bacteriophage in these compositions have a concentration of at least 4×10¹² phage/ml, or at least 1×10¹⁴ phage/ml. The EU/phage ratio of the composition is less than 1×10⁻¹⁰ EU/phage, less than 1×10⁻¹¹ EU/phage, less than 1×10⁻¹² EU/phage, less than 1×10⁻¹³ EU/phage, or less than 5×10⁻¹⁴ EU/phage.

In some aspects of the invention, the compositions comprise less than 20 ng/mL bacterial cell DNA, and less than 10 ng/mL bacterial cell protein (also referred to as host cell protein or HCP).

In some embodiments, the large-scale compositions of this invention may be concentrated or converted to a solid form for subsequent reconstitution by methods well known in the art, such as ultrafiltration, evaporation, spray-drying, lyophilization, etc. When such methods are applied and the resulting form is still liquid, the concentrations of bacteriophage and endotoxin (and in some cases, bacterial cell DNA and bacterial cell protein) will increase, but the ratio of endotoxin to bacteriophage will remain approximately the same as in the large scale composition. When such methods are applied and the resulting form is solid, the ratio of bacteriophage to endotoxin will remain approximately the same as in the large scale composition. Such solid form or concentrated compositions are also part of the present invention.

In certain embodiments, the invention provides pharmaceutically acceptable compositions comprising filamentous bacteriophage having an EU/phage ratio of less than 5×10⁻¹⁴ EU/phage. Pharmaceutically acceptable compositions may, for example, be in the form of a saline solution.

In some embodiments, the invention provides pharmaceutically acceptable compositions in single dosage forms. In some aspects, single dosage forms comprise a portion of the large-scale pharmaceutical composition of the invention. The ratio of endotoxin to bacteriophage will remain approximately the same in the single dosage form as in the large-scale composition. Single dosage forms may be in a liquid or a solid form. Single dosage forms may be administered directly to a patient without modification or may be diluted or reconstituted prior to administration. In certain embodiments, the single dosage forms contain less than 200 endotoxin units, less than 100 endotoxin units, less than 50 endotoxin units, less than 20 endotoxin units, less than 10 endotoxin units, less than 8 endotoxin units, less than 5 endotoxin units, less than 3 endotoxin units, less than 2 endotoxin units, less than 1 endotoxin units, less than 0.5 endotoxin units, or less than 0.2 endotoxin units.

In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, such as by infusion, or via an implanted pump, such as an ICV pump. In the latter embodiment, the single dosage form may be an infusion bag or pump reservoir pre-filled with the indicated number of filamentous bacteriophage. Alternatively, the infusion bag or pump reservoir may be prepared just prior to administration to a patient by mixing a single dose of the filamentous bacteriophage with the infusion bag or pump reservoir solution.

In some embodiments, when administered to a human patient, the pharmaceutically acceptable composition or single dosage form thereof provides less than 5.0 endotoxin units per kilogram body weight per dose. In a more specific aspect of this embodiment, when administered to a human patient, the pharmaceutically acceptable composition or single dosage form thereof provides less than 0.2 endotoxin units per kilogram body weight per dose.

In one embodiment, the pharmaceutical compositions described above are prepared by admixing all or a portion of the large-scale composition with at least one pharmaceutically acceptable excipient. Accordingly, methods for preparing a pharmaceutical composition of filamentous bacteriophage comprising admixing a portion of the large-scale composition comprising filamentous bacteriophage with at least one pharmaceutically acceptable excipient are also encompassed.

In certain embodiments, the pharmaceutical compositions are further subjected to dilution or concentration; or to tabletting, lyophilization, direct compression, melt methods, or spray drying to form tablets, granulates, nano-particles, nano-capsules, micro-capsules, micro-tablets, pellets, or powders.

Single dosage forms of the pharmaceutical composition of the invention may be prepared by portioning the large-scale composition or the pharmaceutical composition into smaller aliquots or into single dose containers or formulating the large-scale composition or the pharmaceutical composition into single dose solid forms, such as tablets, granulates, nano-particles, nano-capsules, micro-capsules, micro-tablets, pellets, or powders. Containers for the smaller aliquots or the single dose containers include vials, infusion bags and pump reservoirs. Vials contemplated for single dose include 1 ml vials, 2 ml vials, 3 ml vials, 5 ml vials, 10 ml vials, 20 ml vials, 30 ml vials, 40 ml vials, 50 ml vials, 60 ml vials, 70 ml vials, 80 ml vials, 90 ml vials, and 100 ml vials. Vials may contain a single dose in a liquid form or a solid form. Vials containing a single dose in a solid form may be reconstituted by adding liquid, typically sterile water or saline solution, prior to administration to a patient. Vials containing a single dose in a liquid form are typically filled with the filamentous bacteriophage composition or pharmaceutical composition at 50% to 90% of the vial volume or from 60% to 80% of the vial volume.

In some embodiments, compositions according to the invention comprise an amount of endotoxin that when administered to a human provides less than 5.0 endotoxin units per kilogram body weight per dose, or less than 0.2 endotoxin units per kilogram body weight per dose. For purposes of this calculation, the human may be assumed to have a weight of at least 40 kg or 50 kg, and the dose may be assumed to have a maximum volume of 10 mL for liquid dosage forms. The dose may be for administration as a bolus (e.g., an injection) or over an amount of time of up to 1 hour (e.g., an infusion). Accordingly, single dosage forms according to the invention can comprise less than 250 endotoxin units; less than 200 endotoxin units; less than 10 endotoxin units; less than 8 endotoxin units; less than 25 endotoxin units per mL; less than 20 endotoxin units per mL; less than 1 endotoxin unit per mL; or less than 0.8 endotoxin units per mL. Multiple dosage forms according to the invention can comprise less than 250 endotoxin units per dose; less than 200 endotoxin units per dose; less than 10 endotoxin units per dose; less than 8 endotoxin units per dose; less than 25 endotoxin units per mL per dose; less than 20 endotoxin units per mL per dose; less than 1 endotoxin unit per mL per dose; or less than 0.8 endotoxin units per mL per dose.

Further embodiments of the invention include:
a composition comprising filamentous bacteriophage according to the invention and an endotoxin that when administered to a human provides less than 5.0 endotoxin units per kilogram body weight per dose, wherein the human has a body weight of at least 40 kg and the dose has a maximum volume of 10 mL;
a composition comprising filamentous bacteriophage according to the invention and an endotoxin that when administered to a human provides less than 0.2 endotoxin units per kilogram body weight per dose, wherein the human has a body weight of at least 40 kg and the dose has a maximum volume of 10 mL;
a composition comprising filamentous bacteriophage according to the invention and an endotoxin that when administered to a human provides less than 5.0 endotoxin units per kilogram body weight per dose, wherein the human has a body weight of at least 50 kg and the dose has a maximum volume of 10 mL; and
a composition comprising filamentous bacteriophage according to the invention and an endotoxin that when administered to a human provides less than 0.2 endotoxin units per kilogram body weight per dose, wherein the human has a body weight of at least 50 kg and the dose has a maximum volume of 10 mL.

Another aspect of the invention includes methods for preparing a pharmaceutical composition of the invention wherein the method comprises subjecting the large scale composition or the pharmaceutical composition to tabletting, lyophilization, direct compression, melt methods, or spray drying to form tablets, granulates, nano-particles, nano-capsules, micro-capsules, micro-tablets, pellets, or powders.

Formulating the large-scale composition or the pharmaceutical composition into nano-particles, nano-capsules, micro-capsules, micro-tablets, pellets, or powders that are subsequently put into capsules is likewise encompassed.

In some embodiments, compositions according to the invention are wild-type filamentous bacteriophage or filamentous bacteriophage which do not display an antibody or a non-filamentous bacteriophage antigen on its surface. The filamentous bacteriophage can be any filamentous bacteriophage such as M13, f1, or fd. Any filamentous bacteriophage is expected to behave and function in a similar manner as they have similar structure and as their genomes have greater than 95% genome identity. In some embodiments, the compositions according to the invention do not comprise a filamentous bacteriophage which displays an antibody on its surface. In some embodiments, the compositions according to the invention do not comprise a filamentous bacteriophage which displays a non-filamentous bacteriophage antigen on its surface.

Purification Methods

Purification methods for obtaining the compositions of the invention are also encompassed and are described in detail below. Utilizing these methods allows for a percent recovery of bacteriophage of at least 10%, preferably 30, 40, 50, 60, or 70%.

Exemplary Purification Procedures

Filamentous bacteriophage to be purified according purification methods according to the invention are obtained in solution, for example, in culture media, after growth in gram-negative bacteria. In some aspects of the invention, the filamentous bacteriophage are obtained according to the exemplary processes described in U.S. application Ser. No. 61/512,169, filed Jul. 27, 2011, incorporated herein in its entirety.

As a general matter, the purification methods according to the invention can comprise a series of chromatography steps. Exemplary steps and combinations of steps are provided below.

In some embodiments, the methods comprise providing bacteriophage material that has been subjected to one or more steps such as centrifugation, nuclease treatment, an/or filtration.

In some embodiments, nuclease treatment was or can be performed before or during the filtration step, for example as described in Examples 10 and 11 below, respectively.

In some embodiments, the methods comprise at least one hydrophobic interaction chromatography step.

In some embodiments, the methods comprise at least one anion exchange chromatography step, which may be a reductive or binding-type step. (In reductive steps, the bacteriophage material is not retained on the column for a wash step but rather progresses through the column; this type of step is commonly run isocratically until the product has been collected. In binding type-steps, the bacteriophage material is loaded onto the column and is eluted by a buffer that tends to reduce the interaction of the bacteriophage material with the column matrix relative to the strength of interaction in loading buffer.) In some embodiments, the methods comprise at least two anion exchange chromatography steps. When at least two anion exchange chromatography steps are used, it is possible for one step to be a binding anion exchange step and the other to be a reductive anion exchange step.

In some embodiments, the material loaded onto a column for one or more of the chromatography steps comprises detergent. For an exemplary list of detergents compatible with bacteriophage, see Example 13. In some embodiments, the column loaded with material comprising detergent is an anion exchange column. The bacteriophage can be incubated with the detergent for a period before column loading, for example, 1 hour. The chromatography step following loading with material comprising detergent can be a binding-type step or reductive-type step.

In some embodiments, the methods comprise at least one chromatography step using a cationically charged polyamine-based resin that binds endotoxin. The resin for this step can be Etoxiclear resin (available from ProMetic BioSciences Ltd., Rockville, Md., USA).

Etoxiclear columns are characterized by the manufacturer as follows:

Mean particle size of 100±10 μm

Cross-linked 6% near-monodisperse agarose (PuraBead 6XL)

Dynamic binding capacity >500,000 EU/mL of adsorbent (loading at 120 cm/hr, 5 minute residence time)

Maximum operational flow rate of up to 400 cm/hr (5 mL Pre-Packed EtoxiClear Column)

Recommended operational flow rate of up to 200 cm/hr

Operational pH range of pH 4.0 to pH 8.0.

Centrifugation

A starting volume of filamentous bacteriophage in solution are centrifuged for a time and speed sufficient to separate the filamentous bacteriophage from bacterial cells and bacterial cell by-products in the starting solution, such as, for example, cellular material from the E. coli cells in which the bacteriophage are grown. In one exemplary embodiment, a starting solution of filamentous bacteriophage is centrifuged at about 4000 rpm for 40 minutes at between 2 and 8° C. in a Sorvall RC-3 centrifuge, or the like, using a Sorvall HG 4 L rotor, or the like. After centrifugation, the supernatant is collected and the pellet is discarded.

DNase Treatment

The supernatant may next be treated with a DNase enzyme for a time and at a concentration sufficient to degrade any E. coli cellular DNA that may be present. In one exemplary embodiment, 0.5-1 L of supernatant from the centrifuge step above is incubated with the DNase enzyme Benzonase at a concentration of 10 units/mL in the presence of 5 mM MgCl₂. The supernatant and DNase enzyme are incubated in a shake flask at room temperature for about 60 minutes and agitated at a speed of 95 rpm. The benzonase step can be performed before or directly after the centrifugation step, or in some embodiments after the depth filtration step.

Depth Filtration

The DNase-treated supernatant is next subjected to depth filtration, which involves passing the supernatant across at least three filters containing various filter media in series and collecting the flow through, which comprises the filamentous bacteriophage. Depth filtration (in contrast to surface filtration) generally refers to a “thick” filter that captures particulate matter and contaminating organisms based on size, hydrodynamic diameter and structure that are greater than the nominal cut-off of the membrane or membranes (for multiple filters operated in series). Depth filtration materials and methods are well known to one of skill in the art. For example, the filter material is typically composed of a thick and fibrous structure made of, for example, Poly Ether Sulfone (PES) or Cellulose Acetate (CA) with inorganic filter aids such as diatomaceous earth particles embedded in the openings of the fibers. This filter material has a large internal surface area, which is key to particle capture and filter capacity. Such depth filtration modules contains pores of from 1.0 μm to 4.5 μm, including filter sizes of at least 1.0, 1.5, 2.0, 2.5, 3.0, 3.5, 4.0 and 4.5 μm, and fractional filter sizes between. Exemplary depth filtration modules include, but are not limited to, Whatman Polycap HD modules (Whatman Inc.; Florham Park, N.J.), Sartorius Sartoclear P modules (Sartorius Corp.; Edgewood, N.Y.) and Millipore Millistak HC modules (Millipore; Billerica, Mass.). In one particular embodiment, the cell culture fluid is clarified via depth filtration (performed at room temperature) and the filamentous bacteriophage are recovered in the filtrate.

In some embodiments, depth filtration is carried out before DNAse treatment.

In one exemplary embodiment, depth filtration of 0.5-1 L occurs across three filters in series. The solution from the centrifugation step or DNase treatment step is passed over each filter with a peristaltic pump. In each case the flow through is collected. The filters may be as follows:

TABLE 1
Exemplary Depth Filtration Filters

| | |
|---|---|
| Sartopure GF + 1.2 μm | Filter is operated according to the |
| (Sartorius), 500 cm2 | manufacturers recommendations |
| | (50-150 mL/min) |
| Sartopure GF + 0.65 μm, | Filter is operated according to the |
| 1000 cm2 | manufacturers recommendations |
| | (100-150 mL/min) |
| Sartopore 2 XLG, 2000 cm2 | Filter is operated according to the |
| | manufacturers recommendations |
| | (100-150 mL/min) |

This series of filtration sub-steps serves to clarify and reduce bioburden. An increase in scale can be achieved by increasing the membrane surface area (e.g., larger filters) or a greater number of smaller filters.

Ultrafiltration and Diafiltration

After the final depth filtration step, the flow through is applied to an ultrafiltration/diafiltration step, where the filamentous bacteriophage are retained by the membrane (500 or 750 KD NMWCO). The goal of diafiltration is to complete buffer exchange, and the goal of the ultrafiltration is purification, or removal of components having a molecular weight lower than 500 or 750 KDa. In one exemplary embodiment, 500 mL of clarified supernatant+/−benzonase treatment is diafiltered using a Poly Ether Sulfone (“PES”) 500 or 750 KD Net Molecular Weight Cut Off (“NMWCO”) against 5-10 volumes of 25 mM Tris, 100 mM NaCl, pH 8.0. Alternatively, the clarified supernatant is diafiltered against 5-10 volumes of 25 mM Tris, 100 mM NaCl, pH 7.4. The cross flow, or transmembrane pressure (dP) is about 5 psi. The permeate rate is set at about 100 mL/min. Filamentous bacteriophage, such as, for example, M13, are retained by the membrane (“the retentate fraction”), and the permeate passes across the membrane.

The ultrafiltration/diafiltration step may also be referred to as “ultrafiltration (UF)”, or “tangential flow filtration (TFF)”.

In some embodiments, the material coming off of the TFF step (i.e., the ultrafiltration/diafiltration step) is depth filtered using, for example, a Sartoguard PES Capsule 0.2 μm (Sartorius), 0.021 m² at a manufacturers recommended flowrate of 150 mL/min.

HIC Phenyl

Material derived from the TFF step is loaded in a high salt buffer (e.g., 2-2.1 M NaCl) onto a 3 L column containing Toyopearl Phenyl 650M (Tosoh Bioscience) with a bed height about 21 cm. This is achieved by diluting 2 fold (1:1 dilution) with 25 mM Tris-HCl 4M NaCl pH 7.4 or the like. The column is pre-equilibrated with about 3 column volumes (“CV”) of 25 mM Tris-HCl pH 7.4, 2M NaCl or the like at a linear flowrate of 97.5 cm/h. Typically, 300-500 mL of filamentous bacteriophage in solution at a concentration of at least 4×10¹² phage/mL are loaded onto the column at a linear flowrate of 48.7 cm/h. This is followed by a wash step of about 3 CV of 25 mM Tris-HCl pH 7.4, 2 M NaCl at a linear flowrate of 97.5 cm/h. The phage fraction is eluted in 3 CV of 25 mM Tris-HCl pH 7.4 250 mM NaCl or the like at a linear flowrate of 97.5 cm/h. The filamentous bacteriophage peak is collected (typically 2-2.5 L) based on inline detection. Filamentous bacteriophage are eluted in a step or linear gradient. When using a step gradient, there is a sharp decrease to 250 mM NaCl rather than a gradual linear gradient to change the NaCl concentration. The column step yield is typically 90% or greater for M13. Similar yields are expected with other filamentous bacteriophage. The purpose of this step is to increase product purity by decreasing host cell contaminants through hydrophobic interaction chromatography (Functional group Phenyl) run in bind and elute mode. In other embodiments a linear gradient may be used.

In order to ensure consistent collection of the peak and to provide a starting point and end point for peak collection, peak collection criteria is based on fluorescence or absorbance (this is also useful when transferring the process step between sites to ensure that the same peak collection parameters are applied). The absorbance is typically detected in real time after flowing through the column. Further analysis on peak fractions can provide further (more specific and supplemental) information regarding where and how much of the product has eluted from the column (e.g. off line ELISA). The product enriched fraction can also be tested off line for contaminants such as endotoxin.

Fluorescence detection (excitation wavelength—242 nm, emission wavelength—334 nm) provides a sensitive method to detect filamentous bacteriophage such as M13. Alternatively, filamentous bacteriophage can also be detected by absorbance using a wavelength of 254 nm or 280 nm (A269 nm). For fluorescence detection, the peak is usually collected starting at 0.1 U (fluorescence units) or 0.05 AU (absorbance units) at A254 nm or 0.01 AU (absorbance units) on the leading edge (upward slope) and collection is stopped on the trailing edge (downside slope) of the peak. In one embodiment, collection is started upon observing a peak, an increase that can be less or greater than 1% of the peak height at the expected retention time or volume and collection is stopped when the signal drops to about 5% of the maximum peak height. In a further embodiment, peak collection is started at a defined process time (based on the expected elution time or elution volume). In one exemplary embodiment, collection may begin and end at an absorbance unit of 0.05 to 0.05 U (254 nm) and 0.01 to 0.01 U (280 nm). Other absorbance wavelengths and emission wavelengths may also used.

The column is stripped with 3 CV of 25 mM Tris-HCl pH 7.4 2M NaCl or the like followed by a NaOH wash of the matrix (CIP).

Weak Anion Exchange Resin (e.g., DEAE AEX)

Next, the eluate fraction from the preceding Phenyl HIC step is diluted with about 5 volumes of 25 mM Phosphate pH 6.5 buffer or the like and filtered through a weak anion exchange resin, such as, for example, a Sartopore 2, 150, 0.45 μm/0.2 μm filter or the like. The pH is typically pH 6.0-7.0, including 6.5, and the conductivity 16.8 mS/cm. In one exemplary embodiment, the 3 L column (bed height circa 22 cm) is equilibrated with 3 CV of 25 mM Phosphate 100 mM NaCl pH 6.5 at a linear flowrate of 97.5 cm/h. The filamentous bacteriophage fraction from the previous step (diluted and filtered as described above) is loaded at a flowrate of 97.5 cm/h. The column is washed with 2 CV of 25 mM Phosphate 150 mM NaCl pH 6.5 followed by 4 CV with 25 mM Phosphate 250 mM NaCl pH 6.5, the wash steps are run at a flowrate of 97.5 cm/h. Filamentous bacteriophage are eluted with 3 CV of 25 mM Phosphate 300mM NaCl pH 6.5 at a flowrate of 97.5 cm/h or the like. The phage peak is collected (typically 3-3.5 L) based on in-line detection of fluorescence and/or absorbance. In-line detection is detection in real time after flowing through the column. Further analysis on peak fractions can provide further (more specific and supplemental) information regarding where and how much of the product has eluted from the column (e.g. off line ELISA). The product enriched fraction can also be tested off line for contaminants such as endotoxin.

Fluorescence detection (excitation wavelength—242 nm, emission wavelength—334 nm) provides a sensitive method to detect filamentous bacteriophage such as M13. Alternatively, filamentous bacteriophage can also be detected by absorbance using a wavelength of 254 nm or 280 nm (A269 nm). For fluorescence detection, the peak is usually collected starting at 0.1 U (fluorescence units) or 0.05 AU (absorbance units) at A254 nm or 0.01 AU (absorbance units) on the leading edge (upward slope) and collection is stopped on the trailing edge (downside slope) of the peak. In one embodiment, collection is started upon observing a peak, an increase that can be less or greater than 1% of the peak height at the expected retention time or volume, and collection is stopped when the signal drops to about 5% of the maximum peak height. In a further embodiment, peak collection is started at a defined process time (based on the expected elution time or elution volume). In one exemplary embodiment, collection may begin and end at an absorbance unit of 0.05 to 0.05 U (254 nm) and 0.01 to 0.01 U (280 nm). Other absorbance wavelengths and emission wavelengths may also used.

The column is stripped with 3 CV of 25 mM Phosphate 1M NaCl pH 6.5 or the like followed by a NaOH wash of the matrix (CIP).

Filamentous bacteriophage are eluted in a step or linear gradient. When using a step gradient, the column step yield is typically 55% or greater for M13. Other filamentous bacteriophage are expected to have similar yields. The purpose of this step is to increase product purity by decreasing host cell contaminants through weak anion exchange (functional group diethylaminoethyl (DEAF)) chromatography run in bind and elute mode.

Strong Anion Exchange Resin (e.g., AEX Q)

The M13 eluate from the weak anion exchange resin (e.g., DEAE) is diluted with an equal volume (1:1) of 25 mM Tris pH 7.4 or the like, and filtered across a suitable filter, such as, for example, a Sartopore 300 0.45+0.2 μm filter (Sartorius). The pH is typically 7.3 and the conductivity 15.8 mS/cm. In one embodiment, a Source 15Q (GE Healthcare) column is equilibrated with 3 CV of 20 mM Tris-HCl pH 7.4 or the like at a linear flowrate of 169.5 cm/h. Filamentous bacteriophage, such as, for example, M13, is loaded at 169.5 cm/h. The column is washed with 3 CV of 25 mM Tris 200 mM NaCl pH 7.4 or the like. Filamentous bacteriophage, such as, for example, M13, are eluted with 5 CV of 25 mM Tris-HCl pH 7.4, 280 mM or 300 mM NaCl (or the like) at a flowrate of 169.5 cm/hr. The phage peak is collected (typically 0.5 L) based on in-line detection. The absorbance or fluorescence is typically detected in real time after flowing through the column. Further analysis on peak fractions can provide further (more specific and supplemental) information regarding where and how much of the product has eluted from the column (e.g. off line ELISA). The product enriched fraction can also be tested off line for contaminants such as endotoxin.

Fluorescence detection (excitation wavelength—242 nm, emission wavelength—334 nm) provides a sensitive method to detect filamentous bacteriophage such as M13. Alternatively, filamentous bacteriophage can also be detected by absorbance using a wavelength of 254 nm or 280 nm. For fluorescence detection, the peak is usually collected starting at 0.1 U (fluorescence units) or 0.05 AU (absorbance units) at A254 nm or 0.01 AU (absorbance units) on the leading edge (upward slope), and collection is stopped on the trailing edge (downside slope) of the peak. In one embodiment, collection is started upon observing a peak, an increase that can be less or greater than 1% of the peak height at the expected retention time or volume and collection is stopped when the signal drops to about 5% of the maximum peak height. In a further embodiment, peak collection is started at a defined process time (based on the expected elution time or elution volume). In one exemplary embodiment, collection may begin and end at an absorbance unit of 0.05 to 0.05 U (254 nm) and 0.01 to 0.01 U (280 nm). Other absorbance wavelengths (e.g., A269 nm) and emission wavelengths may also used.

The column is stripped with 3 CV of 25 mM Phosphate 1M NaCl pH 7.4 followed by a NaOH wash of the matrix (CIP).

Filamentous bacteriophage are eluted in a step or linear gradient. When using a step gradient, the column step yield is typically 80% or greater for M13. Other filamentous bacteriophage are expected to have similar yields. The purpose of this step is to increase product purity by decreasing host cell contaminants through strong anion exchange (Functional group Quaternary Ammonium (Q)) chromatography run in bind and elute mode.

Mustang Q/Clearance Filter

The eluate from the previous step (strong anion exchange resin; AEX Q) is loaded directly onto one or more 10 mL Mustang Q (Pall) membrane at a flowrate of about 150 mL/min. “Mustang Q” may also be referred to herein as “clearance filter,” or “final clearance filter.” A Sartobind filter (Sartorious) may be used in place of a Mustang Q filter. The charged filter (functional group Q) is operated in “flow through” mode. The filamentous bacteriophage product (e.g., M13) containing flow through fraction is collected. This step serves to remove remaining negatively charged contaminants, which are primarily endotoxin, but may also remove host cell DNA and negatively charged host cell proteins.

Ultrafiltration

Filamentous bacteriophage, such as, for example, M13, are concentrated and diafiltered into PBS (155 mM NaCl, 1.06 mM KH2PO4, 2.97 mM Na2HPO4.7H2O—pH7.4) using a 500 kD NMWCO PES hollow fiber filter.

The system is washed with approximately 5 system volumes (25 mL) of water followed by 5 system volumes (25 mL) of 0.5 M NaOH (50° C.). 0.5 NaOH is re-circulated over the filter for about 20 to 40 minutes. The NaOH is removed by a 5 system volume wash with Water for Injection (WFI) water or the like followed by a five system volume wash with 25 mM Tris 280 mM NaCl pH 7.4 or the like. The product (M13 flow through from the previous Mustang Q process step) is added to the system and concentrated to target concentration of about 1.0-1.5×10¹⁴ phage/mL, circulated and diafiltered by the addition of 5-10 volumes of Phosphate Buffered Saline (PBS) pH 7.4.

Typically, the yield for M13 after this step is 70% or greater. Other filamentous bacteriophage are expected to have similar yields.

Sterile Filtration

The supernatant recovered from the ultrafiltration step is filtered across one or more Whatman PURADISC 25 filters or Sartoscale Sartopore 2, 0.2 μm (or the like) at an approximate rate of 2 mL/min, or any other suitable flow rate. The concentration post filtration is adjusted to the target concentration of, for example, 4×10¹² phage/mL, or in some embodiments 1.0×10¹⁴ phage/mL, or 1.0×10¹³ phage/mL with Phosphate Buffered Saline pH 7.4.

TABLE 2
Exemplary Process Steps

| Step | Short | |
|---|---|---|
| # | Description | Details |
| 1 | Centrifugation | Centrifuging culture media comprising |
| | | filamentous bacteriophage for a time |
| | | and speed sufficient to separate cellular |
| | | material from the supernatant. |
| | | Example: 4000 rpm, 40 minutes, 2-8° C. |
| | | in a Sorvall RC-3 with a Sorvall HG 4 L |
| | | rotor. |
| | | supernatant is collected and cell pellet |
| | | is discarded. |
| 2 | DNase | treating the supernatant with a DNase |
| | Treatment* | enzyme thereby facilitating DNA |
| | *steps 2 and | removal by generating smaller |
| | 3 may be | fragments and nucleotides. In the event |
| | reversed. | that DNase treatment precedes depth |
| | | filtration, facilitates passage across |
| | | depth filters. |
| | | Example: 0.5-1 L of culture |
| | | supernatant or TFF centrate (where |
| | | steps 2 and 3 are reversed) is incubated |
| | | in a 2 L flask with Benzonase at a |
| | | concentration of 10 units/mL in the |
| | | presence of 5 mM MgCl₂. This is |
| | | incubated at a shaker speed of 95 rpm |
| | | at room temperature for 60 minutes. |
| 3 | Depth | Applying the DNase-treated supernatant |
| | Filtration* | to depth filtration, and collecting the |
| | *steps 2 and | flow through comprising the filamentous |
| | 3 may be | bacteriophage. Further purposes of |
| | reversed. | clarification/particulate reduction. |
| | | Example: Depth filtration of 0.5-1 L |
| | | (as an example) occurs across three |
| | | filters in series, material is passed over |
| | | each filter with a peristaltic pump. In |
| | | each case the flow through is collected. |
| | | The filters may be as follows: |
| | | (1) Sartopure GF + 1.2 μm (Sartorius), |
| | | 500 cm² |
| | | Filter is operated according to the |
| | | manufacturers recommendations (50- |
| | | 150 mL/min) |
| | | (2) Sartopure GF + 0.65 μm, 1000 cm² |
| | | Filter is operated according to the |
| | | manufacturers recommendations (100- |
| | | 150 mL/min) |
| | | (3) Sartopore 2 XLG, 2000 cm² |
| | | Filter is operated according to the |
| | | manufacturers recommendations (100- |
| | | 150 mL/min) |
| 4 | Ultra- | Ultrafiltering to reduce any low |
| | filtration | molecular weight contaminants such as |
| | and Dia- | host cell proteins, spent fermentation |
| | filtration | media contaminants, digested host cell |
| | | DNA, and DNase enzyme using 500 |
| | | KDa or 750 KDa net molecular weight |
| | | cut off membrane (“NMWCO”); and |
| | | diafiltering to exchange the buffer. |
| | | (<500 KD/<750 KD) and buffer |
| | | exchange (diafiltration). |
| | | Example: Ultrafiltration - 500 mL of |
| | | clarified supernatant +/− benzonase |
| | | treatment is diafiltered using a 500 or |
| | | 750 KD NMWCO Poly Ether Sulfone |
| | | (PES) filter against 5-10 volumes of 25 |
| | | mM Tris, 100 mM NaCl, pH 8.0. The |
| | | cross flow pressure dP is 5 psi. The |
| | | permeate rate is set at 100 mL/min. |
| | | M13 is retained by the membrane (the |
| | | retentate fraction), and the permeate |
| | | passes across the membrane. |
| | | Diafiltration - the retentate fraction |
| | | is depth filtered using a Sartoguard |
| | | PES Capsule 0.2 μm (Sartorius), 0.021 |
| | | m² at a manufacturers recommended flowrate |
| | | of 150 mL/min. |
| 5 | Phenyl | Applying the diafiltered retentate |
| | 650M HIC | fraction from step 4 to a |
| | | chromatography column comprising HIC |
| | | Phenyl in order to purify phage from |
| | | contaminants. This step is based on |
| | | hydrophobic interaction chemistry. |
| | | Example: Phenyl HIC |
| | | Material derived from the diafiltration of |
| | | step 4 is loaded in a high salt buffer (2- |
| | | 2.1M NaCl) onto a 3 L column (bed |
| | | height circa 21 cm) containing Toyopearl |
| | | Phenyl 650M, Tosoh Bioscience). This |
| | | is achieved by diluting 2 fold (1:1 |
| | | dilution) with 25 mM Tris-HCl 4M NaCl |
| | | pH 7.4. The column is pre-equilibrated |
| | | with 3 column volumes (CV) of 25 mM |
| | | Tris-HCl pH 7.4, 2M NaCl at a linear |
| | | flowrate of 97.5 cm/h. Typically 400-500 |
| | | mL of material at a concentration of at |
| | | least 4 × 10¹² phage/mL are loaded onto |
| | | the column at a linear flowrate of 48.7 |
| | | cm/h. This is followed by a wash step of |
| | | 3 CV of 25 mM Tris-HCl pH 7.4, 2M |
| | | NaCl at a linear flowrate of 97.5 cm/h. |
| | | The phage fraction is eluted in 3 CV of |
| | | 25 mM Tris-HCl pH 7.4 250 mM NaCl at |
| | | a linear flowrate of 97.5 cm/h. The |
| | | phage peak is collected (typically 2- |
| | | 2.5 L) based on fluorescence and/or |
| | | absorbance. The column is stripped |
| | | with 3 CV of 25 mM Tris-HCl pH 7.4 2M |
| | | NaCl followed by a NaOH wash of the |
| | | matrix (CIP). |
| | | M13 is eluted in a step gradient, i.e., for |
| | | example, there is a sharp increase to |
| | | 250 mM NaCl rather than a gradual |
| | | linear gradient to change the NaCl |
| | | concentration. |
| 6 | DEAE | applying the collected material from |
| | AEX | step 5 to DEAE AEX, to purify phage |
| | | from contaminants based on anion |
| | | exchange chemistry |
| | | Example: The eluate fraction from the |
| | | preceding Phenyl 650M HIC Step is |
| | | diluted with 5 volumes of 25 mM |
| | | Phosphate pH 6.5 buffer and filtered |
| | | through a Sartopore 2 150 .45 μm/.2 |
| | | μm filter. The pH of the sample fraction |
| | | is typically pH 6.3-6.4 and the |
| | | conductivity 16.8 mS/cm. The 3 L |
| | | column (bed height circa 22 cm) is |
| | | equilibrated with 3 CV of 25 mM |
| | | Phosphate 100 mM NaCl pH 6.5 at a |
| | | linear flowrate of 97.5 cm/h. The phage |
| | | fraction from the previous step (diluted |
| | | and filtered as described above) is |
| | | loaded at a flowrate of 97.5 cm/h. The |
| | | column is washed with 2 CV of 25 mM |
| | | Phosphate 150 mM NaCl pH 6.5 |
| | | followed by 4 CV with 25 mM Phosphate |
| | | 250 mM NaCl pH 6.5, the wash steps |
| | | are run at a flowrate of 97.5 cm/h. M13 |
| | | is eluted with 3 CV of 25 mM Phosphate |
| | | 300 mM NaCl pH 6.5 at a flowrate of |
| | | 97.5 cm/h. The phage peak is collected |
| | | (typically 3-3.5 L) based on in-line |
| | | detection. The column is stripped with 3 |
| | | CV of 25 mM Phosphate 1M NaCl pH |
| | | 6.5 followed by a NaOH wash of the |
| | | matrix (CIP). |
| 7 | AEX | applying the collected material from |
| | Q | step 6 to strong anion exchange |
| | | (Functional group Quaternary |
| | | Ammonium (Q)) chromatography run in |
| | | bind and elute mode, in order to purify |
| | | phage from contaminants based on |
| | | anion exchange chemistry |
| | | Example: Circa 1.5 L of DEAE eluate is |
| | | diluted with an equal volume (1:1) of |
| | | 25 mM Tris pH 7.4 is filtered across a |
| | | Sartopore 300 .45 + .2 μm filter |
| | | (Sartorius). The pH is of the load is |
| | | typically 7.3 and the conductivity 15.8 |
| | | mS/cm. The 200 mL Source 15Q (GE |
| | | Healthcare) column is equilibrated with |
| | | 3 CV of 25 mM Tris-HCl pH 7.4 at a |
| | | linear flowrate of 169.5 cm/h. M13 is |
| | | loaded also at 169.5 cm/h. The column |
| | | is washed with 3 CV of 20 mM Tris 250 |
| | | mM NaCl pH 7.4. M13 is eluted with 5 |
| | | CV of 25 mM Tris-HCl pH 7.4, 280 or |
| | | 300 mM NaCl at a flowrate of 169.5 |
| | | cm/hr. The phage peak is collected |
| | | (typically 0.5 L) based on in-line |
| | | detection The column is stripped with 3 |
| | | CV of 25 mM Phosphate 1M NaCl pH |
| | | 7.4 followed by a NaOH wash of the |
| | | matrix (CIP). |
| 8 | Mustang | applying the collected material from |
| | Q | step 7 to a Mustang Q Filter, in order to |
| | | purify phage from contaminants based |
| | | on anion exchange chemistry |
| | | Example: The eluate from the previous |
| | | step (circa 0.5 L) is loaded directly onto |
| | | one or more 10 mL Mustang Q (Pall) |
| | | membrane at a flowrate of 150 mL/min. |
| | | The charged filter (functional group Q) |
| | | is operated in “flow through” mode. The |
| | | product (M13) containing flow through |
| | | fraction is collected. This step serves to |
| | | remove remaining negatively charged |
| | | contaminants, this is primarily endotoxin |
| | | (but also has the potential to take out |
| | | host cell DNA and negatively charged |
| | | host cell proteins) |
| 10 | Ultra- | Ultrafiltration, to concentrate and buffer |
| | filtration | exchange (diafilter) into the final |
| | | formulation buffer at a target |
| | | concentration at or above the final |
| | | desired product concentration |
| | | Example: M13 is concentrated and |
| | | diafiltered into PBS using a 500 kD |
| | | NMWCO PES hollow fiber filter. |
| | | The system is washed with 5 system |
| | | volumes (25 mL) of water followed by |
| | | system volumes (25 mL) of 0.5M NaOH |
| | | (50° C.). 0.5 NaOH is re-circulated over |
| | | the filter for 30 min. The NaOH is |
| | | removed by a 5 system volume wash |
| | | with Hi-clone water followed by a five |
| | | system volume wash with 25 mM Tris |
| | | 280 mM NaCl pH 7.4. the product is |
| | | added to the system and circulated, and |
| | | followed by concentration to 1.0-1.5 × |
| | | 10¹⁴ phage/mL and then diafiltered by |
| | | the addition of 5-10 volumes of |
| | | Phosphate Buffered Saline (PBS) pH |
| | | 7.4. The concentration is checked by |
| | | measuring the absorbance at 269 nm, |
| | | further concentration as needed can be |
| | | applied at this stage to reach the |
| | | required final target concentration of |
| | | 1.0-1.5 × 10¹⁴ phage/mL. |
| | | The filamentous bacteriophage from the |
| | | Mustang Q step may be split into |
| | | batches for this phage, i.e., the |
| | | bacteriophage product may be equally |
| | | divided in three sub-lots and run |
| | | through the ultrafiltration process in |
| | | parallel. |
| 11 | Sterile | sterile filter the supernatant |
| | Filtration | Example: The supernatant is filtered |
| | | across one or more Whatman |
| | | PURADISC 25, 0.22 μm or Sartoscale |
| | | Sartopore 2, 0.22 μm (Sartorius) filter |
| | | at an approximate rate of 2 mL/min. The |
| | | concentration post filtration is adjusted |
| | | to the target concentration of 1.0 × 10¹⁴ |
| | | phage/mL or between 1.0 × 10¹⁴ and |
| | | 1.5 × 10¹⁴ phage/mL with Phosphate |
| | | Buffered Saline (PBS) pH 7.4. |

The following is a list of exemplary embodiments of phage purification methods according to the invention.
1. A method for preparing a composition comprising filamentous bacteriophage and less than 1×10⁻¹⁰ endotoxin units per filamentous bacteriophage comprising;
   a) providing a first loading buffer comprising filamentous bacteriophage, wherein the filamentous bacteriophage were centrifuged, treated with a nuclease, and filtered after the filamentous bacteriophage were grown;
   b) performing a first chromatography step comprising contacting a first chromatography resin with the first loading buffer comprising the filamentous bacteriophage, contacting the resin with fresh buffer, and collecting a first elution fraction comprising the filamentous bacteriophage;
   c) performing a second chromatography step comprising contacting a second chromatography resin with a second loading buffer comprising the previously collected filamentous bacteriophage, contacting the resin with fresh buffer, and collecting a second elution fraction comprising the filamentous bacteriophage;
   d) performing a final chromatography step comprising contacting a final chromatography resin with a final loading buffer comprising the filamentous bacteriophage, contacting the resin with fresh buffer, and collecting a final elution fraction comprising the filamentous bacteriophage and less than 1×10⁻¹⁰ endotoxin units per filamentous bacteriophage,
      wherein at least one of the chromatography steps is an anion exchange step.
2. The method of embodiment 1 above, wherein the nuclease treatment of the preparation occurred prior to a filtration step.
3. The method of embodiment 1 above, wherein the nuclease treatment of the preparation occurred during a filtration step.
4. The method of any one of embodiments 1 to 3 above, wherein the final elution fraction comprises less than 1×10⁻¹¹ endotoxin units per filamentous bacteriophage.
5. The method of any one of embodiments 1 to 4 above, wherein the final elution fraction comprises less than 1×10⁻¹² endotoxin units per filamentous bacteriophage.
6. The method of any one of embodiments 1 to 5 above, wherein the final elution fraction comprises less than 1×10⁻¹³ endotoxin units per filamentous bacteriophage.
7. The method of any one of embodiments 1 to 6 above, wherein the final elution fraction comprises less than 5×10⁻¹⁴ endotoxin units per filamentous bacteriophage.
8. The method of any one of embodiments 1 to 7 above, wherein the filamentous bacteriophage comprise phage that do not display an antibody or a non-filamentous bacteriophage surface antigen.
9. The method of any one of embodiments 1 to 8 above, wherein the filamentous bacteriophage comprise wild-type phage.
10. The method of any one of embodiments 1 to 9 above, wherein the filamentous bacteriophage comprise M13 phage.
11. The method of any one of embodiments 1 to 10 above, wherein the first chromatography resin comprises a hydrophobic interaction chromatography resin or an anion exchange resin.
12. The method of any one of embodiments 1 to 11 above, wherein the second chromatography resin comprises an anion exchange resin.
13. The method of any one of embodiments 1 to 12 above, wherein the first or second chromatography resin comprises a weak anion exchange resin.
14. The method of embodiment 13 above, wherein, before contacting the weak anion exchange resin with a loading buffer, a detergent is added to the loading buffer.
15. The method of embodiment 14 above, wherein the detergent is chosen from Triton X-100 and Zwittergent Z3-12.
16. The method of any one of embodiments 13 to 14 above, wherein the detergent is present at a concentration ranging from 0.05% to 2%.
17. The method of any one of embodiments 1 to 16 above, wherein the final chromatography resin comprises an anion exchange resin.
18. The method of any one of embodiments 1 to 17 above, wherein the final chromatography resin comprises a cationically charged polyamine-based resin that binds endotoxin.
19. The method of embodiment 18 above, wherein the affinity resin is Etoxiclear resin.
20. The method of any one of embodiments 1 to 19 above, wherein the first chromatography resin comprises a weak anion exchange resin and the first chromatography step is performed as a reductive chromatography step in the presence of a detergent, the second chromatography resin comprises a weak anion exchange resin and the second chromatography step is performed as a binding chromatography step, and the final chromatography resin comprises a cationically charged polyamine-based resin that binds endotoxin.
21. The method of any one of embodiments 1 to 19 above, wherein the first chromatography resin comprises a hydrophobic interaction chromatography resin, the second chromatography resin comprises a weak anion exchange resin and the second chromatography step is performed as a binding chromatography step, and the final chromatography resin comprises a cationically charged polyamine-based resin that binds endotoxin.
22. The method of any one of embodiments 1 to 19 above, further comprising, between the second and final chromatography steps, performing an additional chromatography step comprising contacting an additional chromatography resin with an additional loading buffer comprising the previously collected filamentous bacteriophage, contacting the additional resin with fresh buffer, and collecting a second elution fraction comprising the filamentous bacteriophage.
23. The method of embodiment 22 above, wherein the first chromatography resin comprises a hydrophobic interaction chromatography resin, the second chromatography resin comprises a weak anion exchange resin and the second chromatography step is performed as a reductive chromatography step in the presence of a detergent, the additional chromatography resin comprises a weak anion exchange resin and the additional chromatography step is performed as a binding chromatography step, and the final chromatography resin comprises a cationically charged polyamine-based resin that binds endotoxin.
24. The method of embodiment 22 above, wherein the first chromatography resin comprises a hydrophobic interaction chromatography resin, the second chromatography resin comprises a weak anion exchange resin and the second chromatography step is performed as a binding chromatography step, the additional chromatography resin comprises a strong anion exchange resin and the additional chromatography step is performed as a binding chromatography step, and the final chromatography resin comprises a strong anion exchange resin and the final chromatography step is performed as a reductive chromatography step.
25. The method of any one of embodiments 1 to 19 above, wherein the method yields phage particles in an amount of at least 10% relative to input as measured by OD or ELISA.
26. The method of any one of embodiments 1 to 25 above, wherein the final elution fraction comprises at least 10¹³ phage particles.
27. The method of any one of embodiments 1 to 26 above, wherein the final elution fraction comprises phage particles at a concentration of at least 10¹² per mL as measured by OD or ELISA.
28. The method of any one of embodiments 1 to 27 above, further comprising formulating the bacteriophage obtained from the final chromatography step and at least one pharmaceutical excipient into a pharmaceutical composition.
29. A method for purifying a culture of filamentous bacteriophage comprising:
   a) centrifuging culture media comprising filamentous bacteriophage for a time and speed sufficient to separate cellular material from the supernatant;
   b) treating the supernatant of the centrifuged media with a DNase enzyme;
   c) applying the supernatant from step b) to depth filtration;
   d) ultrafiltering the depth filtered supernatant with a 500 or 700 KDa molecular weight cut off membrane and diafiltering the retentate to exchange the buffer;
   e) applying the diafiltered retentate to a chromatography column comprising HIC Phenyl;
   f) applying the M13 elution fraction from the HIC Phenyl column to a chromatography column comprising a weak anion exchange resin;
   g) applying the M13 elution fraction from the weak anion exchange resin to a strong anion exchange resin;
   h) applying the M13 elution fraction from the strong anion exchange resin to a filter clearance step;
   i) ultrafiltering the flow through, concentrating and diafiltering against the final formulation buffer; and
   j) sterile filtering the retentate.

Formulations

Techniques for formulation of drugs may be found, for example, in “Remington's Pharmaceutical Sciences,” Mack Publishing Co., Easton, Pa., latest edition, which is incorporated herein by reference in its entirety.

Suitable routes of administration for the pharmaceutical compositions of the invention may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternatively, one may administer a pharmaceutical composition in a local rather than systemic manner, for example, via injection of the pharmaceutical composition directly into the brain of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into compositions which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological compositions for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose compositions such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG) . If desired, disintegrating agents may be added, such as cross-linked polyvinyl pyrrolidone, agar, or alginic acid or a salt thereof such as sodium alginate.

In one embodiment, tablets, granulates, nano-particles, nano-capsules, micro-capsules, micro-tablets, pellets, or powders are encompassed, either uncoated or enterically coated. The nano-particles, nano-capsules, micro-capsules, micro-tablets, pellets, or powders may be put into capsules.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the filamentous bacteriophage of the present invention are conveniently delivered in the form of an aerosol spray from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The compositions described herein may be formulated for parenteral administration, e.g., by bolus injection or continuous infusion. Formulations for injection may be presented in unit dosage form, e.g., in vials, ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the filamentous bacteriophage in water-soluble form. Additionally, suspensions of the active ingredients may be prepared as oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents (e.g., surfactants such as polysorbate (Tween 20)) which increase the solubility of the active ingredients to allow for the preparation of highly concentrated solutions. A protein based agent such as, for example, albumin may be used to prevent adsorption of M13 to the delivery surface (i.e., IV bag, catheter, needle, etc.).

Alternatively, the filamentous bacteriophage may be in powder form for constitution with a suitable vehicle, e.g., sterile, pyrogen-free water based solution, before use.

The pharmaceutical compositions of the present invention may also be formulated in rectal compositions such as suppositories or retention enemas, using, e.g., conventional suppository bases such as cocoa butter or' other glycerides.

### Filamentous Bacteriophage for Use in Treatment and Methods of Treatment Comprising Administering Filamentous Bacteriophage

In some embodiments, the invention provides a filamentous bacteriophage composition according to the invention for use in treating a plaque-forming disease, for reducing the amount of amyloid plaque in a patient suffering from a plaque-forming disease, for inhibiting the formation of amyloid deposits or for disaggregating pre-formed amyloid deposits, or for reducing susceptibility to a plaque-forming disease.

In some embodiments, the invention provides methods for treating a plaque-forming disease, for inhibiting the formation of amyloid deposits or for disaggregating pre-formed amyloid deposits in a patient, for reducing the amount of amyloid plaque in a patient suffering from a plaque-forming disease, or for reducing susceptibility to a plaque-forming disease, each of which comprise administering a filamentous bacteriophage composition according to the invention to a patient in need thereof.

In certain aspects of these embodiments, the filamentous bacteriophage provided in the uses and methods according to the invention does not display any non-filamentous bacteriophage antigen on its surface. In certain aspects of these embodiments, the filamentous bacteriophage provided in the uses and methods according to the invention is a wild-type bacteriophage. In a more specific aspect, the bacteriophage is a wild-type bacteriophage. In an even more specific aspect, the filamentous bacteriophage is selected from M13, f1, or fd. Each of these filamentous bacteriophage is expected to behave and function in a similar manner as they have similar structure and their genomes have greater than 95% genome identity. In an even more specific embodiment, the filamentous bacteriophage used in the methods and compositions for the uses described above according to the present invention is wild-type M13.

In certain aspects of these embodiments, the plaque-forming disease is selected from early onset Alzheimer's disease, late onset Alzheimer's disease, presymptomatic Alzheimer's disease, SAA amyloidosis, hereditary Icelandic syndrome, senility, multiple myeloma, to prion diseases that are known to affect humans (such as for example, kuru, Creutzfeldt-Jakob disease (CJD), Gerstmann-Straussler-Scheinker disease (GSS), and fatal familial insomnia (FFI)) or animals (such as, for example, scrapie and bovine spongiform encephalitis (BSE)), Parkinson's Disease, Argyrophilic grain dementia, Corticobasal degeneration, Dementia pugilistica, diffuse neurofibrillary tangles with calcification, Down's syndrome, Frontotemporal dementia with parkinsonism linked to chromosome 17, Hallervorden-Spatz disease, Myotonic dystrophy, Niemann-Pick disease type C, Non-Guamanian motor neuron disease with neurofibrillary tangles, Pick's disease, Postencephalitic parkinsonism, Progressive subcortical gliosis, Progressive supranuclear palsy, Subacute sclerosing panencephalitis, and Tangle only dementia. In more specific aspects of these embodiments, the plaque-forming disease is selected from early onset Alzheimer's disease, late onset Alzheimer's disease or pre-symptomatic Alzheimer's disease.

Methods involving disaggregating pre-formed amyloid deposits may comprise directly contacting any of the filamentous bacteriophage compositions of the invention with the pre-formed amyloid deposits.

In one aspect of methods according to the invention, the bacteriophage is administered to the patient as part of a pharmaceutically acceptable composition additionally comprising a pharmaceutically acceptable carrier. For example, the pharmaceutically acceptable carrier can be saline.

In one embodiment of methods according to the invention, the filamentous bacteriophage composition is administered intranasally. In one embodiment of compositions for the uses described above, the filamentous bacteriophage composition is formulated for intranasal administration.

In another embodiment of methods according to the invention, the filamentous bacteriophage are administered directly to the brain of the subject. Administration “directly to the brain” includes injection or infusion into the brain itself, e.g., intracranial administration, as well as injection or infusion into the cerebrospinal fluid. In one aspect of this embodiment, administration is by intrathecal injection or infusion, intraventricular injection injection or infusion, intraparenchymal injection or infusion, or intracerebroventricular injection or infusion. In more specific aspects, administration is by intraparenchymal injection; intracerebroventricular injection; or intracerebroventricular infusion. In one embodiment of compositions for the uses described above, the filamentous bacteriophage composition is formulated for administration directly to the brain of a subject, such as by intracranial administration, as well as injection or infusion into the cerebrospinal fluid, intrathecal injection or infusion, intraventricular injection injection or infusion, intraparenchymal injection or infusion, or intracerebroventricular injection or infusion.

Methods delineated herein also include those wherein the patient is identified as in need of a particular stated treatment. Identifying a patient in need of such treatment can be in the judgment of a patient or a health care professional and can be subjective (e.g., opinion) or objective (e.g., measurable by a test or diagnostic method).

It is to be understood that both the foregoing and following description are exemplary and explanatory only and are not restrictive of the invention, as claimed.

### EXAMPLE 1

### Exemplary Production Process Steps of the Invention

Tables 3 through 13 show in table format exemplary specifications for purification processes according to the invention. Those of skill in the art will know where modifications may be made without compromising the novel methods described herein.

TABLE 3
Upstream Process
Centrifugation Step

| Function | Parameter | Requirements |
|---|---|---|
| Centrifuge | Speed (rpm) | 4,000 |
| (1 L Bottles) | Time (min) | 40 |
| | Temperature (° C.) | |

Sorvall RC-3 with a Sorvall HG 4 L rotor

TABLE 4
Exemplary Filtration Specifications

| | | |
|---|---|---|
| Function | Parameter | Requirements |

Supernatant Filtration: Sartopure GF + 1.2 μm

| | | |
|---|---|---|
| Maximum scale | Membrane areas | 500 |
| performed | (cm²) | |
| previously | | |
| Filter | Filter type | Glass Fiber Fleeces |
| | | (Prefilter) |
| | Throughput L/m² | 95.3 |
| | Flowrate mL/min | 50-150 |
| | Pressure | |

Supernatant Filtration: Sartopure GF + 0.65 μm

| | | |
|---|---|---|
| Maximum scale | Membrane areas | 1000 |
| performed | (cm²) | |
| previously | | |
| Filter | Filter type | Glass Fiber Fleeces |
| | | (Prefilter) |
| | Capacity L/m² | 45.2 |
| | Flowrate mL/min | 100-150 |
| | Pressure | |

Supernatant Filtration: Sartopore 2 XLG

| | | |
|---|---|---|
| Maximum scale | Membrane areas | 2000 |
| performed | (cm²) | |
| previously | | |
| Filter | Filter type | Polyethersulfone |
| | | (Final Filter) |
| | Capacity L/m² | 24.4 |
| | Flowrate mL/min | 30-150 |
| | Pressure | |
| Other | | The filter clogs if the |
| | | cells do not pellet during |
| | | centrifugation. |

TABLE 5
Exemplary DNase Treatment
Benzonase Step

| Function | Parameter | Requirements |
|---|---|---|
| DNA | Benzonase Concentration | 10 |
| Digestion | (units/mL) | |
| | Magnesium Chloride | 5 |
| | Concentration (mM) | |
| DNA | Shake Speed (rpm) | 95 |
| Digestion | Time (min) | 60 |
| | Flask size (L) | 2 |
| | Sample Volume (L) | 1 |
| | Temperature (C.) | Ambient (room |
| | | temperature) |

The benzonase step can be performed before or directly after the centrifugation step, or after the three stage depth filtration.

TABLE 6
Exemplary Diafiltration Steps
Diafiltration Step

| Function | Parameter | Requirements |
|---|---|---|
| Buffer | Tris (mM) | 25 |
| | Sodium Chloride (mM) | 100 |
| | pH | 8.0 or 7.4 |
| Cross | Pressure (psi) | 5 |
| Flow | Permeate Rate (mL/min) | 100 |
| | Diafiltration Volumes | 5-10 |
| | Membrane type (hollow fibre | Hollow Fiber |
| | or flat sheet cassette) | |
| | Filter material (PES, | Poly Sulfone |
| | Reg Cellulose) | |
| | Filter MW cut-off (e.g. | 750 kDa or 500 kDa |
| | 30 kDa) | |
| | Membrane Area (cm²) | 280 |

TABLE 7
Exemplary Filtration Steps
Filtration: Sartoguard PES Capsule 0.2 μm

| Function | Parameter | Requirements |
|---|---|---|
| Maximum scale | Membrane area | .021 |
| performed | (m²) | |
| previously | | |
| Filter | Filter type | Polyethersulfone |
| | Capacity L/m² | |
| | Flowrate mL/min | 150 |
| | Pressure | |

TABLE 8
Exemplary Chromatography Step
Chromatography Step 1 - Phenyl HIC Column

| Function | Parameter | Requirements | |
|---|---|---|---|
| Maximum scale | Column size | 3 | L |
| performed | | | |
| previously | | | |
| Column | Media type | Phenyl-650M | |
| | Binding capacity (phage/mL) | 5.5 × 10¹² | |
| | Bed height (cm) | 21 | |
| | Net pore size (microns) | | |
| | Linear flowrate (cm/hr) | 97.46 | cm/hr |
| | or residence time (min) | | |

| | | |
|---|---|---|
| Packing | Pressure or | Flow |
| technique | flow pack | |

| | | |
|---|---|---|
| Flowrate | 250 | mL/min |

| | |
|---|---|
| Pressures | Less than 50 psi. |
| Packing buffer | 25 mM Tris-HCl |
| | pH 7.4 2M NaCl |

| | | | |
|---|---|---|---|
| Pre run column | Sanitization solution | 0.5M | NaOH |

| CIP | CIP method (including number | | 3 column volumes with a 30 |
|---|---|---|---|
| | of CV's/hold times) | | minute hold time after 1 and |
| | | | ½ column volumes. |
| | | | Wash with MilliQ water until |
| | | | the pH is below pH 8.0. |
| | | | Charge column with 2 CV |
| | | | Tris-HCl pH 7.4 2M NaCl and 4 |
| | | | CV 25 mM Tris-HCl pH 7.4 |
| Load preparation | | Setup | Sample is diluted 1:1 with |
| (insert pH adjustment/ | | | 25 mM Tris-HCl 4M NaCl |
| dilution step table | | | pH 7.4 |
| if required) | | Capacity | 400 mL of 10¹³ M13 |
| | | (g/m² or L/m²) | as determined by ELISA |

| | | |
|---|---|---|
| Flowrate | 125 | mL/min |

| | |
|---|---|
| Sample pH | 7.42 |
| Sample Conductivity | 150 |
| (mS/cm) | |

| Column run | Equilibration (CV) | 3 CV of 25 mM Tris-HCl | |
|---|---|---|---|
| (include contingency | | pH 7.4 2M NaCl | |
| with CV stated) | Linear flowrate (cm/hr) | 97.46 | cm/hr |
| | or residence time (min) | | |
| | Sample loading linear flowrate | 48.73 | cm/hr |
| | (cm/hr) or residence time (min) | | |
| | Wash Out unbound (CV) | 3 CV of 25 mM Tris-HCl | |
| | | pH 7.4, 2M NaCl | |
| | Wash Out unbound linear flowrate | 97.46 | cm/hr |
| | (cm/hr) or residence time (min) | | |
| | Elution gradient or step | Step | |
| | Elution details (CV) | 3 CV of 25 mM Tris-HCl | |
| | | pH 7.4 250 mM NaCl | |
| | Elution linear flowrate (cm/hr) | 97.46 | cm/hr |
| | or residence time (min) | | |
| | Strip (CV) | 3 CV of 25 mM Tris-HCl | |
| | | pH 7.4 2M NaCl | |
| | Strip linear flowrate (cm/hr) | 97.46 | cm/hr |
| | or residence time (min) | | |
| | Product collection criteria | Fluorescence: | |
| | (include start and stop | Ex. 242 Em. 334 | |
| | mAU where required) | 0.1-0.1 | |
| | | Absorbance(254 nm): | |
| | | 0.05-0.05 | |
| | | Absorbance(280 nm): | |
| | | 0.01-0.01 | |
| | All cell flow path length | 16 μL flow cell | |
| | Method Duration | 175 | minutes |
| Post run column | Sanitization Solution | 0.5 | NaOH |
| CIP | CIP Method (including number | 3 column volumes with a 30 | |
| | of CV's/hold times) | minute hold time after 1 and | |
| | | ½ column volumes. | |
| | | Wash with MilliQ water until | |
| | | the pH is at 6.5-7.0. Then | |
| | | store column in 20% ethanol. | |
| Processing | Step Yield | ~90% | |
| | Approximate volume of eluate | 2-2.5 | L |
| | (CV) | | |
| Hold step | Temperature | 4° | C. |

| | |
|---|---|
| Duration (include range where | tbd |
| possible) | |

TABLE 9
Exemplary Fractogel DEAE Column
Chromatography Step 2 - Fractogel DEAE Column

| Function | Parameter | Requirements | |
|---|---|---|---|
| Maximum scale | Column size | 3 | L |
| performed | | | |
| previously | | | |
| Column | Media type | Fractogel | |
| | Binding capacity (g/L) | 4.67 × 10¹² | |
| | Bed height (cm) | 22 | |
| | Net pore size (microns) | | |
| | Linear flowrate (cm/hr) | 97.46 | cm/hr |
| | or residence time (min) | | |

| | | |
|---|---|---|
| Packing | Pressure or | Flow |
| technique | flow pack | |

| | | |
|---|---|---|
| Flowrate | 250 | mL/min |

| | |
|---|---|
| Pressures | Less than 50 psi. |
| Packing buffer | 25 mM Phosphate |
| | 100 mM NaCl pH 6.5 |

| | Pack test details | | |
|---|---|---|---|
| | Asymmetry specification | | |
| | Plate number specification | | |
| Pre run column | Sanitisation solution | 0.5M | NaOH |

| CIP | CIP method (including number | | 3 column volumes with a 30 |
|---|---|---|---|
| | of CV's/hold times) | | minute hold time after 1 and |
| | | | ½ column volumes. |
| | | | Wash with MilliQ water until |
| | | | the pH is below 8.0. |
| Load preparation | HIC elution is | Setup | Sample is diluted with 5 |
| (insert pH adjustment/ | filtered before | | volumes of 25 mM Phosphate |
| dilution step table | dilution. | | pH 6.5 buffer. |
| if required) | | Capacity | |
| | | (g/m² or L/m²) | |
| | Filter: | Flowrate | 150 |
| | Sartopore 2 150 | (mL/min) | |
| | .45 μm/.2 μm | Sample pH | ~6.36 |
| | | Sample conductivity | 16.8 |
| | | (mS/cm) | |

| Column run | Equilibration (CV) | 3 CV of 25 mM Phosphate | |
|---|---|---|---|
| (include contingency | | 100 mM NaCl pH 6.5 | |
| with CV stated) | Linear flowrate (cm/hr) | 97.46 | cm/hr |
| | or residence time (min) | | |
| | Sample loading linear flowrate | 97.46 | cm/hr |
| | (cm/hr) or residence time (min) | | |
| | Wash Out unbound (CV) | 2 CV Wash with 25 mM | |
| | | Phosphate 150 mM NaCl | |
| | | pH 6.5. 4 column volume | |
| | | was with 25 mM Phosphate | |
| | | 250 mM NaCl pH 6.5 | |
| | Wash Out unbound linear flowrate | 97.46 | cm/hr |
| | (cm/hr) or residence time (min) | | |
| | Elution gradient or step | Step | |
| | Elution details (CV) | 3 CV of 25 mM | |
| | | Phosphate 300 mM | |
| | | NaCl pH 6.5. | |
| | Elution linear flowrate (cm/hr) | 97.46 | cm/hr |
| | or residence time (min) | | |
| | Strip (CV) | 3 CV of 25 mM | |
| | | Phosphate 1M NaCl | |
| | | pH 6.5. | |
| | Strip linear flowrate (cm/hr) | 97.46 | cm/hr |
| | or residence time (min) | | |
| | Product collection criteria | Fluorescence: | |
| | (include start and stop | Ex. 242 Em. 334 | |
| | mAU where required) | .1-.1 | |
| | | Absorbance(254 nm): | |
| | | .05-.05 | |
| | | Absorbance(280 nm): | |
| | | .01-.01 | |
| | AU cell flowpath length | 16 μL Flow cell | |
| | Method Duration | 150 | minutes |
| Post run column | Sanitization Solution | 0.5 | NaOH |
| CIP | CIP Method (including number | 3 column volumes with a 30 | |
| | of CV's/hold times) | minute hold time after 1 and | |
| | | ½ column volumes. | |
| | | Wash with MilliQ water until | |
| | | the pH is at 6.5-7.0. Then | |
| | | store column in 20% ethanol. | |
| Processing | Step Yield | ~55% | |
| | Approximate volume of eluate | 3-3.5 | L |
| | (CV) | | |
| Hold step | Temperature | 4° | C. |

| | | |
|---|---|---|
| | Duration (include range where | tbd |
| | possible) | |
| Other | | |
| Insert example | | |
| chromatogram | | |

TABLE 10
Exemplary 15Q Column Specifications
Chromatography Step 3 - Source 15Q Column

| Function | Parameter | Requirements | |
|---|---|---|---|
| Maximum scale | Column size | 200 | mL |
| performed | | | |
| previously | | | |
| Column | Media type | Source 15Q | |
| | Binding capacity (g/L) | 1.86 × 10¹³ | |
| | Bed height (cm) | | |
| | Net pore size (microns) | | |
| | Linear flowrate (cm/hr) | 169.5 | cm/hr |
| | or residence time (min) | | |

| | | |
|---|---|---|
| Packing | Pressure or | Flow |
| technique | flow pack | |

| | | |
|---|---|---|
| Flowrate | 15 | mL/min |

| | |
|---|---|
| Pressures | Less than 50 psi. |
| Packing buffer | 25 mM Tris-HCl |
| | pH 7.4 |

| | Pack test details | | |
|---|---|---|---|
| | Asymmetry specification | | |
| | Plate number specification | | |
| Pre run column | Sanitisation solution | 0.5M | NaOH |

| CIP | CIP method (including number | | 3 column volumes with a 30 |
|---|---|---|---|
| | of CV's/hold times) | | minute hold time after 1 and |
| | | | ½ column volumes. |
| | | | Wash with MilliQ water until |
| | | | the pH is at below 8.0. |
| Load preparation | DEAE Elution | Setup | 1500 mL of DEAE sample is |
| (insert pH adjustment/ | material is | | diluted with 1500 mL of 25 |
| dilution step table | filtered with the | | mM Tris pH 7.4 |
| if required) | Sartopore 300 | Capacity | |
| | .45 + .2 μm | (g/m² or L/m²) | |
| | filter. | Flowrate | 150 |
| | | (mL/min) | |
| | | Sample pH | ~7.3 |
| | | Sample Conductivity | 15.8 |
| | | (mS/cm) | |

| Column run | Equilibration (CV) | 3 CV of 25 mM Tris-HCl | |
|---|---|---|---|
| (include contingency | | pH 7.4 | |
| with CV stated) | Linear flowrate (cm/hr) | 169.5 | cm/hr |
| | or residence time (min) | | |
| | Sample loading linear flowrate | 169.5 | cm/hr |
| | (cm/hr) or residence time (min) | | |
| | Wash Out unbound (CV) | 3 CV wash with 25 mM | |
| | | Tris 200 mM NaCl pH 7.4 | |
| | Wash Out unbound linear flowrate | 169.5 | cm/hr |
| | (cm/hr) or residence time (min) | | |
| | Elution gradient or step | Step | |
| | Elution details (CV) | 5 CV of 25 mM Tris-HCl | |
| | | pH 7.4 280 mM (or 300 mM) | |
| | | NaCl | |
| | Elution linear flowrate (cm/hr) | 169.5 | cm/hr |
| | or residence time (min) | | |
| | Strip (CV) | 3 CV with 25 mM Tris HCl, | |
| | | 1M NaCl, pH 7.4 | |
| | Strip linear flowrate (cm/hr) | 169.5 | cm/hr |
| | or residence time (min) | | |
| | Product collection criteria | Fluorescence: | |
| | (include start and stop | Ex. 242 Em. 334 | |
| | mAU where required) | .1-.1 | |
| | | Absorbance(254 nm): | |
| | | .05-.05 | |
| | | Absorbance(280 nm): | |
| | | .01-.01 | |
| | AU cell flowpath length | 16 μL Flow cell | |
| | Method Duration | 240 | minutes |
| Post run column | Sanitisation Solution | 0.5 | NaOH |
| CIP | CIP Method (including number | 3 column volumes with a 30 | |
| | of CV's/hold times) | minute hold time after 1 and | |
| | | ½ column volumes. | |
| Processing | Step Yield | 80.06% | |
| | Approximate volume of eluate | 500 | mL |
| | (CV) | | |
| Hold step | Temperature | 4° | C. |

| | |
|---|---|
| Duration (include range where | Overnight |
| possible) | |

TABLE 11
Exemplary Mustang Q Filtration Steps
Mustang Q Filtration Step

| Function | Parameter | Requirements |
|---|---|---|
| Maximum scale | Membrane volume | 10 |
| performed | (mL) | |
| previously | | |
| Filter | Filter Material | Hydrophillic polyethersulfone |
| | Capacity g/m² | |
| | Flowrate mL/min | 150 mL/min |
| | Pressure | Less than 50 psi |
| Other | | The Source 15Q elution |
| | | material is over the mustang Q |
| | | filter in the exclusion mode. |

TABLE 12
Exemplary Ultrafiltration Steps
Ultra Filtration Step for concentrating the sample and diafiltration

| Function | Parameter | Requirements | |
|---|---|---|---|
| Maximum scale | Membrane area | 41 | cm² |
| performed | | | |
| previously | | | |
| Membrane | Membrane type (hollow | Hollow Fiber | |
| | fibre or flat sheet cassette) | | |
| | Filter material (PES, | Poly Sulfone | |
| | Reg Cellulose) | | |
| | Filter MW cut-off (e.g. | 500 | kDa |
| | 30 kDa) | | |
| | Loading g/m² | | |
| Pre CIP | Sanitisation solution | 25 mM Tris 280 mM NaCl pH 7.4 | |
| | CIP method (include | System is washed with 5 system | |
| | any hold times) | volumes (25 mL) of WFI (Water | |
| | | for Injection) followed by 5 | |
| | | system volumes (25 mL) of 0.5M | |
| | | NaOH (50° C.). 0.5 NaOH is | |
| | | re-circulated over the filter for | |
| | | 30 min. The NaOH is removed | |
| | | by a 5 system volume wash with | |
| | | WFI followed by a five system | |
| | | volume wash with 25 mM Tris | |
| | | 280 mM NaCl pH 7.4. | |
| Processing | TMP | | |
| | Inlet pressure | Less than 50 psi | |
| | Inlet flowrate | 65 | mL/min |
| | Permeate flowrate | 5 | mL/min |
| | Flux (L/m²/hr) | .731 | |
| | Target diafiltration | 1.4 × 10¹⁴ phage/mL, max. | |
| | Concentration (phage/mL) | 1.5 × 10¹⁴ phage/mL | |
| | Diafiltration buffer | 5x sample volume diafiltration | |
| | volume (TOV's) | | |
| | Diafiltration Buffer | 1x Phosphate Buffered Saline | |
| | | pH 7.4 | |
| | Target concentration | 1.4 × 10¹⁴ phage/mL, max. | |
| | (phage/mL) | 1.5 × 10¹⁴ phage/mL | |
| | Buffer flush requirement | System is flushed with 10 mL of 1x | |
| | | PBS to remove any excess phage. | |
| | Final Target | 1 × 10¹⁴ phage/mL, max, | |
| | concentration (g/L) | 1.5 × 10¹⁴ phage/mL | |
| | Processing time | 180 | min |

| | | |
|---|---|---|
| Post CIP | Sanitization solution | 0.5M Sodium Hydroxide |
| | CIP method (include | System is washed with 5 system |
| | any hold times) | volumes (25 mL) of Hi-clone water |
| | | followed by system volumes (25 |
| | | mL) of 0.5M NaOH (50° C.). 0.5 |
| | | NaOH is re-circulated over the filter |
| | | for 30 min. The NaOH is removed |
| | | by a 5 system volume wash with |
| | | Hi-clone water. |
| Processing | Step yield | ~70% |
| Hold Step | Temperature (° C.) | 4 |
| | Duration | Overnight |
| Other | | |

TABLE 13
Exemplary Sterile Filtration Step
Whatman PURADISC 25

| Function | Parameter | Requirements |
|---|---|---|
| Maximum scale | Membrane area | 490 |
| performed | (mm²) | |
| previously | | |
| Filter | Filter type | Polyethersulfone |
| | Capacity g/m² | |
| | Flowrate mL/min | 2 |
| | Pressure | Less than 50 psi |
| Other | | After the sample is filtered |
| | | absorbance concentration |
| | | calculations are determined and |
| | | the sample is diluted with 1x |
| | | PBS buffer pH 7.4 to reach a |
| | | concentration of 1.0 × 10¹⁴ |
| | | phage/mL or other desired |
| | | target concentration. |

### EXAMPLE 2

### Exemplary Buffers and Solutions

TABLE 14
Exemplary buffers

| | | | | | | |
|---|---|---|---|---|---|---|
| Media/ | | | | | Guide pH & | |
| Buffer/Feed | Chemical | | | | Conductivity | Specific |
| Name | Constituents | MW | g/L | Titrant | Range | Requirements |

| | | | | | |
|---|---|---|---|---|---|
| Benzonase | MgCl₂*H₂O | 203.3 | 1.0165 | | |
| MgCl₂ | | | | | |
| Solution | | | | | |
| Fermentation | Tris Base | 121.14 | 3.0285 | HCl | 8.0 |
| Diafiltration | Sodium | 58.44 | 5.844 | | |
| Buffer | Chloride | | | | |
| HIC Column | Tris Base | 121.14 | 3.0285 | HCl | 7.4 |
| Sample | Sodium | 58.44 | 233.76 | | |
| Preparation | Chloride | | | | |
| Buffer | | | | | |
| HIC Column | Tris Base | 121.14 | 3.0285 | HCl | 7.4 |
| Equilibration | Sodium | 58.44 | 116.88 | | |
| Buffer | Chloride | | | | |
| HIC Column | Tris Base | 121.14 | 3.0285 | HCl | 7.4 |
| Wash Buffer | | | | | |
| HIC Column | Tris Base | 121.14 | 3.0285 | HCl | 7.4 |
| Elution Buffer | Sodium | 58.44 | 14.61 | | |
| | Chloride | | | | |
| DEAE | Dibasic Sodium | 268.03 | 2.0 | NaOH | 6.5 |
| Column | Phosphate | | | | |
| Equilibration | Heptahydrate | | | | |
| Buffer | Monobasic | 137.99 | 2.4 | | |
| | Sodium | | | | |
| | Phosphate | | | | |
| | Sodium | 58.44 | 5.844 | | |
| | Chloride | | | | |
| DEAE | Dibasic Sodium | 141.96 | 2.0 | NaOH | 6.5 |
| Column | Phosphate | | | | |
| Wash Buffer | Heptahydrate | | | | |
| 1 | Monobasic | 137.99 | 2.4 | | |
| | Sodium | | | | |
| | Phosphate | | | | |
| | Sodium | 58.44 | 8.766 | | |
| | Chloride | | | | |
| DEAE | Dibasic Sodium | 268.03 | 2.0 | NaOH | 6.5 |
| Column | Phosphate | | | | |
| Wash Buffer | Heptahydrate | | | | |
| 2 | Monobasic | 137.99 | 2.4 | | |
| | Sodium | | | | |
| | Phosphate | | | | |
| | Sodium | 58.44 | 14.61 | | |
| | Chloride | | | | |
| DEAE | Dibasic Sodium | 268.03 | 2.0 | NaOH | 6.5 |
| Column | Phosphate | | | | |
| Wash Buffer | Monobasic | 137.99 | 2.4 | | |
| 3 | Sodium | | | | |
| | Phosphate | | | | |
| | Sodium | 58.44 | 58.44 | | |
| | Chloride | | | | |
| Source 15Q | 25 mM Tris | | | HCl | 7.4 |
| equilibration | | | | | |
| buffer | | | | | |
| Source 15Q | 25 mM Tris, | | | HCl | 7.4 |
| wash buffer | 200 mm NaCl | | | | |
| Source 15Q | 25 mM Tris, | | | HCl | 7.4 |
| elution/ | 280 mm NaCl | | | | |
| Mustang Q | | | | | |
| Buffer | | | | | |
| Source 15Q | 25 mM Tris | | | HCl | 7.4 |
| Strip Buffer | 1M NaCl | | | | |
| Final | 155 mM NaCl, | | | | |
| Formulation | 1.06 mM | | | | |
| Buffer | KH₂PO₄, | | | | |
| (1x PBS pH | 2.97 mM | | | | |
| 7.4) | Na₂HPO₄•7H₂O - | | | | |
| | pH 7.4 | | | | |

### EXAMPLE 3

### Representative Purification Process for M13—Batch 1

A purification process according to the invention was followed according to the steps provided in Table 2 and Example 1 for 0.32 Liters of M13 at a starting concentration of 2.45×10¹³ phage/ml. For Batch 1, the hollow fiber was equilibrated with 1× PBS. Subsequent batches were equilibrated with 25 mM Tris 280 mM NaCl pH 7.4.

Table 15 shows the phage recovery results from this experimental purification, including, for example, the total number of phage recovered after each step of the purification process, as well as the % recoveries.

TABLE 15
Phage Recovery for Batch 1

| | Load | Total | Column | |
|---|---|---|---|---|
| | Total | Phage | Recovery | Total |
| | Phage | Recovered | A269 | Recovery |
| Column | (A269) | (A269) | (%) | (%) |
| 3 L Phenyl-HIC | *7.85E+15 | 6.58E+15 | 83.8 | 83.8 |
| 3 L DEAE | 6.51E+15 | 4.27E+15 | 65.5 | 54.4 |
| AEX Q (Source15Q) | 4.15E+15 | 3.97E+15 | 95.8 | 50.5 |
| Mustang Q | 3.85E+15 | 3.45E+15 | 89.7 | 43.9 |
| Ultrafiltration (UF) | 1.50E+15 | 7E+14 | 46.6 | 8.9 |
| Batch 1 (½ of the | | | | |
| flow through from | | | | |
| the Mustang Q filter) | | | | |
| Ultrafiltration (UF) | 1.50E+15 | 9.5E+14 | 63.2 | 12.1 |
| Batch 2 (½ of the | | | | |
| flow through from | | | | |
| the Mustang Q filter) | | | | |
| Ultrafiltration (UF) | | | | 21 |
| Total | | | | |

Table 16 shows the removal of endotoxin after each step of the purification process for Batch 1. Purified (post second UF step) materials from Batch 1 contain 4.8×10⁻¹³ EU/phage.

TABLE 16
Endotoxin Removal for Batch 1

| | Total | Total | Column | Total |
|---|---|---|---|---|
| | EU | EU | Removal | Removal |
| Column | Load | Elution | (%) | (%) |
| 3 liter Phenyl HIC | 9.98E+09 | 3.86E+08 | 96.1 | 96.1 |
| 3 liter DEAE | 3.82E+08 | 9.00E+04 | 99.9 | 99.99909 |
| 200 ml SOURCE Q | 9.00E+04 | 1.31E+04 | 85.4 | 99.99987 |
| Mustang Q | 1.27E+04 | 5.38E+02 | 95.8 | 99.99999 |
| Ultrafiltration (UF) | 2.43E+02 | 3.5E+02 | −44 | |
| Batch 1 | | | | |
| Ultrafiltration (UF) | 2.43E+02 | 4.43E+2 | −82 | |
| Batch 2 | | | | |
| Ultrafiltration (TFF) | | | | 99.99999 |
| Total | | | | |

### EXAMPLE 4

### Representative Purification Process for M13—Batch 2

A purification process according to the invention was followed according to the steps provided in Table 2 and Example 1 for 0.35 Liters of M13 at a starting concentration of 2.4×10¹³ phage/ml. Table 17 shows the phage recovery results from this experimental purification, including, for example, the total number of phage recovered after each step of the purification process, as well as the % recoveries.

TABLE 17
Phage Recovery for Batch 2

| | | | Step | |
|---|---|---|---|---|
| | Load | Recovery | Recovery | Total |
| | Total Phage | Total Phage | A269 | Recovery |
| Column | (A269) | (A269) | (%) | (%) |
| 3 L Phenyl-HIC | *8.38E+15 | 5.09E+15 | 61 | 61 |
| 3 L DEAE | 4.99E+15 | 4.29E+15 | 86 | 51.3 |
| Source15Q | 4.22E+15 | 3.66E+15 | 86.8 | 43.7 |
| Mustang Q | 3.59E+15 | 3.29E+15 | 91.5 | 39.3 |
| UF | 3.29E+15 | 2.4E+15 | 72.8 | 28.6 |
| Concentration | | | | |

Table 18 shows the removal of endotoxin after each step of the purification process for Batch 2. Purified (post UF step) M13 material from Batch 2 contains 9.2×10⁻¹³ EU/phage. The purity after the HIC Phenyl step is 5.8×10⁻⁸ EU/phage. A 6.3×10⁴ increase in purity (EU/phage) is observed from the DEAE step to the final purified material.

TABLE 18
Endotoxin Removal for Batch 2

| | | | Step | Total |
|---|---|---|---|---|
| | Total EU | Total EU | Removal | Removal |
| Column | Load | Recovery | (%) | (%) |
| 3 liter Phenyl | 3.08E+09 | 2.97E+08 | 90.38 | 90.36883 |
| HIC | | | | |
| 3 liter DEAE | 2.97E+08 | 1.70E+05 | 99.94 | 99.99447 |
| 200 ml | 1.70E+05 | 9.57E+03 | 94.38 | 99.99969 |
| SOURCE Q | | | | |
| Mustang Q | 9.57E+03 | 8.10E+02 | 91.54 | 99.99997 |
| Ultrafiltration | 8.10E+02 | 2.2E+03 | −171 | 99.99992 |
| (TFF) | | | | |
| 3 liter Phenyl | 3.08E+09 | 2.97E+08 | 90.38 | 90.36883 |
| HIC | | | | |
| 3 liter DEAE | 2.97E+08 | 1.70E+05 | 99.94 | 99.99447 |
| 200 ml | 1.70E+05 | 9.57E+03 | 94.38 | 99.99969 |
| SOURCE Q | | | | |
| Mustang Q | 9.57E+03 | 8.10E+02 | 91.54 | 99.99997 |
| Ultrafiltration | 8.10E+02 | 2.2E+03 | −171 | 99.99992 |
| (TFF) | | | | |

Table 19 shows an exemplary certificate of analysis for Batch 2.

TABLE 19
Exemplary Certificate of Analysis - Batch 2

| Assay | Result |
|---|---|
| M13 Concentration (by ELISA) | 1.0 × 10¹⁴ phage/ml |
| M13 Concentration (by Absorbance) | 1.0 × 10¹⁴ phage/ml |
| Endotoxin (by Chromogenic LAL) | 92 EU/ml |
| AEX | 98.7% Main Peak |
| | 1.3% Pre Peak |
| | 0% Post Peak |
| AEX Peak Area | 36932240 UV*sec |
| AEX Peak Height | 1015.119 mV |
| SDS PAGE | Conforms to reference material. |
| | Major band at 5 kDa. |
| Bioburden | Pass (No growth after 5 days) |

### EXAMPLE 5

### Representative Purification Process for M13—Batch 3

A purification process according to the invention was followed according to the steps provided in Table 2 and Example 1 for 0.4 Liters of M13 at a starting concentration of 7.2×10¹³ phage/ml.

Table 20 shows the phage recovery results from this experimental purification, including, for example, the total number of phage recovered after each step of the purification process, as well as the % recoveries.

TABLE 20
Phage Recovery for Batch 3

| | | | | | | Elution | Step | |
|---|---|---|---|---|---|---|---|---|
| | Load | Elution | Load | Elution | Load Total | Total | Phage | Overall |
| | Volume | Volume | Concentration | Concentration | Phage | Phage | Recovery | Phage |
| Sample | (mL) | (mL) | ELISA | ELSA | ELISA | ELISA | ELISA (%) | Recovery |
| Diafiltered | | 410 | | 7.02E+13 | | | | |
| 100 mM NaCl | | | | | | | | |
| HIC Elution | 410 | 2470 | 7.02E+13 | 7.10E+12 | 2.88E+16 | 1.75E+16 | 60.9 | 60.9 |
| DEAE Elution | 2470 | 2300 | 7.10E+12 | 5.41E+12 | 1.75E+16 | 1.24E+16 | 70.9 | 43.2 |
| AEX Q | 2290 | 737.5 | 5.41E+12 | 1.09E+13 | 1.24E+16 | 8.04E+15 | 64.9 | 27.9 |
| (Source15-2) | | | | | | | | |
| Elution 280 1 | | | | | | | | |
| Mustang Q | 727.5 | 727.5 | 1.09E+13 | 2.09E+13 | 7.93E+15 | 1.52E+16 | 191.7 | 52.8 |
| Concentration | 717.5 | 56 | 2.09E+13 | 1.00E+14 | 1.50E+16 | 5.60E+15 | 37.3 | 19.4 |
| UF | | | | | | | | |

Table 21 shows the removal of endotoxin after each step of the purification process for Batch 3.

TABLE 21
Endotoxin Removal for Batch 3

| | | Load | Elution | Load | Elution | | ET |
|---|---|---|---|---|---|---|---|
| | Ave | Volume | Volume | Total | Total | Yield | removal |
| Sample | (EU/ml) | (mL) | (mL) | (EU) | (EU) | (%) | (%) |
| Diafiltered | | | 410 | | | | |
| 100 mM NaCl | | | | | | | |
| HIC Elution | 5.19E+05 | 410 | 2470 | | 1.28E+09 | | |
| DEAE Elution | 7.50E+01 | 2470 | 2300 | 1.28E+09 | 1.73E+05 | 0.013456273 | 99.9 |
| Source15-2 | 6.405 | 2290 | 737.5 | 1.72E+05 | 4.72E+03 | 2.750327511 | 97.2 |
| Elution 280 1 | | | | | | | |
| Mustang Q | 0.8775 | 727.5 | 727.5 | 4.66E+03 | 6.38E+02 | 13.70023419 | 86.3 |
| Concentration | 8.51 | 717.5 | 56 | 6.30E+02 | 4.77E+02 | 75.69175179 | 24.3 |
| UF | | | | | | | |

Table 22 shows an exemplary certificate of analysis for Batch 3. Purified (post UF step) M13 material from batch 3 contains 8.5×10⁻¹⁴ EU/phage. The purity after the HIC Phenyl step is 7.3×10⁻⁸ EU/phage. An 8.6×10⁵ increase in purity (EU/phage) is observed from the DEAE step to the final purified material.

TABLE 22
Exemplary Certificate of Analysis - Batch 3

| Assay | Result |
|---|---|
| M13 Concentration (by ELISA) | 1.1 × 10¹⁴ phage/ml |
| M13 Concentration (by Absorbance) | 1.0 × 10¹⁴ phage/ml |
| Endotoxin (by Chromogenic LAL) | 8.5 EU/ml |
| AEX | 88.4% Main Peak |
| | 8.1% Pre Peak |
| | 3.5% Post Peak |
| AEX Peak Area | 31741100 UV*sec |
| AEX Peak Height | 1015.968 mV |
| SDS PAGE | Conforms to reference material. |
| | Major band at 5 kDa. |
| Bioburden | Pass (No growth after 5 days) |

### EXAMPLE 6

### Representative Purification Process for M13—Batch 4

A purification process according to the invention was followed according to the steps provided in Table 2 and Example 1 for 0.4 Liters of M13 at a starting concentration of 2.2×10¹³ phage/ml.

Table 23 shows the phage recovery results from this experimental purification, including, for example, the total number of phage recovered after each step of the purification process, as well as the % recoveries.

TABLE 23
Phage Recovery for Batch 4

| | | | | Elution | Load | Elution | Step Phage | Overall |
|---|---|---|---|---|---|---|---|---|
| | Load | Elution | Load | Concentration | Total | Total | Recovery | Phage |
| | Vol. | Vol. | Concentration | O.D. 269 | Phage | Phage | A269 | Recovery |
| Sample | (mL) | (mL) | O.D. 269 | (Phage/mL) | (A269) | (A269) | (%) | (%) |
| Diafiltered | | 400 | | 2.2E+13 * | | | | |
| 100 mM NaCl | | | | | | | | |
| HIC Elution | 400 | 1920 | 2.20E+13 | 1.45E+12 | 8.80E+15 | 2.78E+15 | 31.6 | 31.6 |
| DEAE Elution | 1910 | 2020 | 1.45E+12 | 1.56E+12 | 2.77E+15 | 3.15E+15 | 113.8 | 35.8 |
| Source15-2 | 2010 | 600 | 1.56E+12 | 3.53E+12 | 3.14E+15 | 2.12E+15 | 67.5 | 24.1 |
| Elution 280 1 | | | | | | | | |
| Mustang Q | 590 | 590 | 3.53E+12 | 4.12E+12 | 2.08E+15 | 2.43E+15 | 116.7 | 27.6 |
| Concentration | 580 | 20 | 4.12E+12 | 1.00E+14 | 2.39E+15 | 2.00E+15 | 83.7 | 22.7 |
| UF | | | | | | | | |

Table 24 shows the removal of endotoxin after each step of the purification process for Batch 4. Purified (post UF step) M13 material from Batch 4 contains 2.2×10⁻¹² EU/phage. The purity after the HIC Phenyl step is 8.7×10⁻⁸ EU/phage. A 4.0×10⁴ increase in purity (EU/phage) is observed from the DEAE step to the final purified material.

TABLE 24
Endotoxin Removal for Batch 4

| | | | Elution | | Elution | | ET |
|---|---|---|---|---|---|---|---|
| | Ave | Load | Vol | Load Total | Total | Yield | removal |
| Sample | (EU/ml) | Volume | (ml) | (EU) | (EU) | (%) | (%) |
| Diafiltered | * | | 400 | | | | |
| 100 mM NaCl | | | | | | | |
| HIC Elution | 1.26E+05 | 400 | 1920 | | 2.42E+08 | | |
| DEAE Elution | 2.13E+02 | 1910 | 2020 | 2.41E+08 | 4.29E+05 | 0.2 | 99.8 |
| Source15-2 | 1.01E+01 | 2010 | 600 | 4.27E+05 | 6.04E+03 | 1.4 | 98.6 |
| Elution 280 1 | | | | | | | |
| Mustang Q | 6.24E+00 | 590 | 590 | 5.94E+03 | 3.68E+03 | 62 | 38 |
| Concentration UF | 220 | 580 | 20 | 3.62E+03 | 4.40E+03 | 121.6 | 21.6 |

### EXAMPLE 7

### Comparison Purification Process for M13—Utilizing CsCl Purification Methods and Not the Methods of the Invention

Table 25 shows the results for a purification process CsCl purification techniques, and not the inventive techniques described in Table 2 or Example 1. M13 material corresponding to the “CsCl” batch was produced by infection of E. coli JM109 grown in batch culture. M13 containing supernatants were harvested by centrifugation and PEG precipitated. Further purification was achieved by two successive rounds of Cesium Chloride (“CsCl”) density gradient purification (generated by ultracentrifugation).

In contrast to the purities observed for the batches described in Examples 1-6, a CsCl purified batch yielded a purity of only 2.6×10⁻¹⁰ EU/phage.

TABLE 25
Exemplary Certificate of Analysis - CsCl purification methods

| Assay | Result |
|---|---|
| M13 Concentration (by ELISA) | 2.0 × 10¹⁴ phage/ml |
| M13 Concentration (by Absorbance) | 1.3 × 10¹⁴ phage/ml |
| Endotoxin (by Chromogenic LAL) | 33,900 EU/ml |
| AEX | 100% Main Peak |
| | 0% Pre Peak |
| | 0% Post Peak |
| AEX Peak Area | 38522780 UV*sec |
| AEX Peak Height | >1000 mV |
| SDS PAGE | Conforms to reference material. |
| | Major band at 5 kDa. |
| Bioburden | Pass (No growth after 5 days) |

### EXAMPLE 8

### Set Points for Endotoxin Levels

Table 26 below outlines calculations that were made in order to set the draft target endotoxin release specifications for M13.

TABLE 26
Target endotoxin specifications
Max EU concentration permissible in solution = max total EU de-
liverable for lowest anticipated patient weight/volume delivered

| IC, single dose - Total per | | ICV - Total per | |
|---|---|---|---|
| patient/hour | | patient/day | |
| EU/hour | EU/hour | EU/day | EU/day |
| 150 Kg | 40 Kg | 150 Kg | 40 Kg |
| 30 | 8 | 720 | 192 |
| 30 | 8 | 720 | 192 |
| 30 | 8 | 720 | 192 |
| 30 | 8 | 720 | 192 |
| | | | Assumption |
| | | | that 0.2 |
| | | | EU/Kg/hour is |
| | | | permissible |
| | | | (over a 24 |
| | | | hour period) |
| Volume | Concentration | Amount | |
| (mL) | (phage/mL) | (phage) | |
| 0.1 | 1.00E+14 | 1.00E+13 | |
| 1 | 1.00E+14 | 1.00E+14 | |
| 10 | 1.00E+14 | 1.00E+15 | |
| 30 | 1.00E+14 | 3.00E+15 | |
| | EU/mL (single | EU/mg (single | |
| | dose administered | dose administered | |
| IC | in 1 hour or less | in 1 hour or less | |
| volume | for 40 Kg) | for 40 Kg) | |
| 0.1 | 80 | 34.8 | |
| 1 | 8 | 3.5 | Used to set |
| | | | specification |
| 5 | 1.6 | 0.7 | Provided for |
| | | | information |
| | | | purposes only |
| | EU/mL | EU/mg | |
| | (continuous | (continuous | |
| ICV | dose over 24 | dose over 24 | |
| volume | h for 40 Kg) | h for 40 Kg) | |
| 0.1 | 1920 | 834.8 | |
| 1 | 192 | 83.5 | |
| 5 | 38.4 | 16.7 | |
| 10 | 19.2 | 8.3 | |
| 30 | 6.4 | 2.8 | Used to set |
| | | | specification |

Specification based on max anticipated volume (or phage amount) delivered [IC 5 mL, 1 × 10¹⁴ phage/mL] for lowest projected potential patient weight (40 Kg)
Draft target specification set at 5 EU/mL based on the current estimation of potential routes of administration and estimated maximum amounts dosed (worst case)
Target specification may be modified at a later date subject to potential changes to route of administration and the maximum projected amount dosed

### EXAMPLE 9

### Exemplary Drug Substance Specification

Table 27 shows the attributes and specifications for an exemplary drug substance comprising M13 filamentous bacteriophage. This specification covers the purified bulk drug substance.

TABLE 27
Exemplary Drug Substance Specification

| | Testing site/ | |
|---|---|---|
| Attribute | Method | Specification |
| Physical Characteristics | | |
| Color, Appearance and | | Clear to opalescent, |
| Clarity | | colorless to straw yellow |
| | | liquid, no visible particles |
| pH | | 7.0-7.8 |
| Osmolality | | Report result |
| Concentration | | |
| Virus concentration by | | 1.0 × 10¹⁴ ± 0.1 × 10¹⁴ |
| Absorbance (A269 nm)^{a} | | phage/mL |
| Identity and Purity | | |
| Infectivity assay tbd | | Report result as |
| | | infectious units/mL |
| Identity by Western Blot | | Comparable to reference |
| Identity by ELISA | | Report result as |
| | | phage/mL |
| Identity by qPCR | | Report result as copy |
| | | number |
| Identity by SDS-PAGE | | Report Major bands, |
| (reduced, Coomassie | | Comparable to reference |
| stain) | | |
| Purity by AEX | | ≧90% monomer by peak |
| | | area, ≦10% aggregates |
| | | and fragments |
| Purity by Size Exclusion | | Report result as % peak |
| Chromatography (SEC) | | area of main peak, pre- |
| | | peaks and post peaks |
| Potency (activity/binding) | | |
| Subject to the development | | |
| of a suitable, robust and | | |
| reproducible assay | | |
| Impurities | | |
| Host Cell DNA by qPCR | | ≦20 ng/mL |
| Host cell protein | | Report result as ng/mL |
| (ELISA) | | |
| Safety | | |
| Endotoxin (LAL)^{b} | | ≦5 EU/mL |
| Extended bioburden by | | No growth detected after |
| direct transfer method | | 14 days |

^{a}UVabs (A269 nm) is currently the method of choice for determining concentration, other alternative include the product specific ELISA and qPCR, there is a possibility that one of these method replaces the ELISA prior to IND filing.
^{b}Specification subject to change dependent on amount dosed, route of administration and further regulatory input.

Table 28 shows the attributes and specifications for an exemplary drug product comprising M13 filamentous bacteriophage. This specification covers the filled drug product, derived from drug substance by passing over two sterile filters in series followed by filling into glass vials, for example.

TABLE 28
Exemplary Drug Product Specification

| | Testing Site/ | |
|---|---|---|
| Attribute | Method | Specification |
| Physical Characteristics | | |
| Color, Appearance and | | Clear to opalescent, |
| Clarity | | colorless to straw |
| | | yellow liquid, no |
| | | visible particles |
| pH | | 7.0-7.8 |
| Volume in Container | Per cUSP <1> | NLT 0.6 mL/vial |
| Concentration | | |
| Virus concentration by | | 1.0 × 10¹⁴ ± 0.1 × 10¹⁴ |
| Absorbance (A269 nm)^{a} | | phage/mL |
| Identity and Purity | | |
| Infectivity, assay tbd | | Report result as |
| | | infectious |
| | | particles/mL |
| Identity by Western Blot | | Report |
| | | result/comparable to |
| | | reference standard |
| Identity by ELISA | | Report result as |
| | | phage/mL |
| Identity by qPCR | | Report result as copy |
| | | number |
| Identity by SDS-PAGE | | Report Major bands, |
| (reduced, Coomassie | | Comparable to |
| stain) | | reference |
| Purity by SEC | | ≧90% monomer by |
| | | peak area, ≦10% |
| | | aggregates and |
| | | fragments |
| Purity by AEX | | Report % peak area |
| | | of main peak, pre- |
| | | peaks and post peaks |
| Potency (activity/binding) | | |
| Subject to the | | |
| development of a | | |
| suitable, robust and | | |
| reproducible assay | | |
| Impurities | | |
| Host Cell DNA by | | ≦20 ng/mL |
| qPCR | | |
| Host Cell Protein | | Report result as |
| (ELISA) | | ng/mL |
| Safety | | |
| Particulates | Per cUSP 28 | ≧10 μm: ≦6000/vial |
| | <788> | ≧25 μm: ≦600/vial |
| Sterility^{b} | <71> | No growth detected |
| | | after 14 day |
| | | incubation, passes |
| | | USP sterility test |
| Endotoxin by LAL^{c} | | ≦5 EU/mL |

^{a}UVabs (A269 nm) is currently the method of choice for determining concentration, other alternative include the product specific ELISA and qPCR, there is a possibility that one of these method replaces the ELISA prior to IND filing.
^{b}Bacteriostasis and fungistasis will be performed on the first cGMP lot released
^{c}Specification subject to change dependent on amount dosed

### EXAMPLE 10

### Alternative Production Process 70065

This example sets forth an exemplary process according to the invention for purification of filamentous bacteriophage having low endotoxin contamination.

Supernatant containing M13 phage from a 5 L fermentation was provided.

Benzonase treatment: Benzonase was added to the supernatant to achieve a final concentration of 10 units per mL and 1M MgCl₂ added to give a final concentration of 5 mM; the material was incubated for 60 minutes at room temperature. The material was then clarified by depth filtration using 0.6 μm, 0.6/0.2 μm and 0.2 μm ULTA Prime capsules (GE). Only 1993.8 g of material was carried forward at this point due to blockage of the filters.

TFF1 step: The clarified material was diafiltered for 10 turnover volumes (TOV), using a 500 kDa MWCO hollow fibre cartridge (0.48 m²) until the pH and conductivity of the permeate was comparable to the diafiltration buffer (25 mM Tris, 100 mM NaCl, pH 8.0). The inlet pressure (˜5 psi) was maintained throughout the diafiltration. The recovered retentate (1864.6 g) was 0.45/0.2 μm filtered (1795.6 g) and sampled for analysis with the remaining bulk stored at 2-8° C.

HIC step: The post TFF1 material (1792.3 g) was diluted 1:1 with 25 mM Tris, 4M NaCl, pH 7.4, 0.2 μm filtered (3739.5 g) and sampled for analysis. The material was at pH 8.0 and had a conductivity of 152.9 mS following dilution.

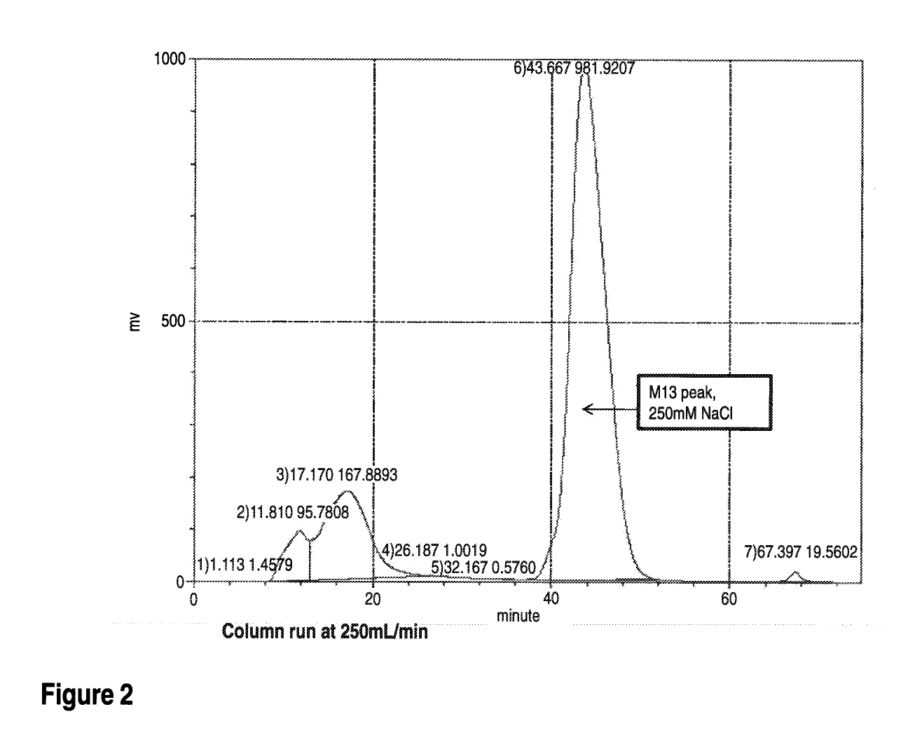

A Toyopearl Phenyl 650M Vantage 90 column (1144.5 mL column volume (CV)) was sanitised prior to use and equilibrated with 25 mM Tris-HCl, 2M NaCl, pH 7.4. The diluted sample (3739.5 g) was loaded onto the Toyopearl Phenyl 650M column at a flow rate of 48.7 cm/hr (50.5 mL/min). The flow through (F/T) unbound material was washed out with 3 CV of 25 mM Tris-HCl, 2M NaCl, pH 7.4, before the NPT002 material was eluted with 250 mM NaCl and the column striped with 2M NaCl. All steps were performed at a flow rate of 97.5 cm/hr (101 mL/min). The phage peak was collected as a single pool (776.1 g) starting from when the A254 increased from baseline and stopped when the peak decreased to baseline (FIG. 2). The product peak was sampled for analysis and stored at 2-8° C. overnight before performing the DEAE step.

DEAE Anion Exchange Step: The post HIC material (772.1 g) was removed from 2-8° C. storage diluted with 5 volumes of 25 mM phosphate pH6.5 and filtered through a 0.45/0.2 μm filter (4614.5 g). The material was at pH 6.05 and had a conductivity of 21.1 mS following dilution. A Fractogel EMD DEAE (M) Vantage 90 column (864.6 mL column volume (CV) was sanitised prior to use and equilibrated with 25 mM phosphate, 100 mM NaCl pH 6.5. The diluted post HIC material (4607.8 g) was loaded onto the column at a now rate of 97.5 cm/hr (101 mL/min). The column was then washed with buffer containing 150 mM NaCl followed a wash at 250 mM NaCl. It was noted that the 150 mM NaCl wash buffer had a conductivity of 17.7 mS which was lower than the conductivity of the sample (21.1 mS). The phage were eluted with 300 mM NaCl and collected as a single pool (1107.8 g) starting from when the absorbance at 254 nm (A₂₅₄) increased from baseline and stopped when the peak decreased to 5% of baseline. The product peak was sampled for analysis and stored at 2-8° C. overnight before performing the Source 15Q step.

Source 15Q step: Post DEAE material (1102.5 g) was removed from 2-8° C. storage, diluted 1:1 with 25 mM Tris-HCl pH7.4, and filtered through a 0.45/0.2 pm filter (2189.9 g). The material was at pH 6.79 and had a conductivity of 15.29 mS following dilution. A Source 15Q Fineline 35 column (182.4 mL column volume (CV)) was sanitised prior to use and equilibrated with 25 mM Tris-HCl, pH 7.4. The diluted post DEAE sample (2183.5 g) and 1 CV of wash buffer containing 200 mM NaCl was loaded onto the column at a flow rate of ˜60 cm/hr (9.5 mL/min). This reduced flow rate was used due to the small bead size of the media and the upper limit of pressure provided by the chromatography system.

The remaining wash step and elution of the phage (in buffer containing 280 mM NaCl) was performed at 169.5 cm/hr (27.1 mL/min) using the AKTA Pilot system pump with the manual system outlet flow path. The eluted material was collected as a single pool (295.9 g) starting from when the A₂₅₄ increased from baseline and stopped when the peak decreased to 5% of baseline. The product peak was sampled for analysis and stored at 2-8° C. overnight before performing the Mustang Q step.

Mustang Q step: A 10 mL Mustang Q capsule was prepared as per the manufacturer's instructions and equilibrated in 25 mM Tris, 280 mM NaCl, pH7.4. The post Source 15Q pool (291.3 g) was removed from 2-8° C. storage and loaded onto the Mustang Q capsule followed by a flush with −50 mL buffer at a flow rate of 150 mL/min. The material was collected as a single pool from start of loading until end of flush (333.5 g). The material was sampled for analysis with the samples stored.

TFF2 step: The initial filter cartridge was found to give a low flow rate, so after the post Mustang Q pool (330.5 g) was initially concentrated approximately 2.8-fold using the 500 kDa MWCO hollow fibre cartridge (0.0041 m²) was initially concentrated approximately 2.8-fold using a 500 kDa MWCO hollow fibre cartridge (0.0041 m²), the retentate (116.8 g) was recovered and the TFF system was rinsed with ˜47 mL of formulation buffer. The TFF retentate was filtered using a Sartopore 2 150 0.45/0.2 μm filter (108.01 g). The material was sampled and the TFF 2 intermediate bulk stored at 2˜8° C. for 7 days. A replacement hollow fiber was obtained, flushed, and wetted out so that its permeability was 474 LMH/barg; then it was sanitised and ready for use. The TFF 2 intermediate bulk material was concentrated to ˜1.1×10¹⁴ particles/mL (˜30 mL) as determined by UV analysis. The material was then buffer exchanged for 6 tum over volumes (TOV) into formulation buffer. The material was then further concentrated to ˜1.5×10¹⁴ particles/mL before being recovered from the system (20.21 g).

The material was sampled and then 0.2 μm filtered using 5×Whatman Puradisc 0.2 μm PES 25 mm syringe filters (Cat no 6780-2502). The material was then diluted with formulation buffer based on UV analysis to give 25 mL at a concentration of 9.93×10¹³ virions/mL by UV.

Data collected during the process are shown in the following table.

TABLE 29
Phage and endotoxin concentrations measured
at various stages of process 70065.

| | | | Endotoxin | |
|---|---|---|---|---|
| | | | (EU per 10¹⁴ | Endotoxin |
| | ELISA | Endotoxin | phage | fold reduction |
| Sample | (phage/mL) | (EU/mL) | particles) | by step |
| Post HIC | 8.2 × 10¹² | 1.92 × 10⁶ | 2.34 × 10⁷ | — |
| DEAE Load | 1.2 × 10¹² | — | — | — |
| Post DEAE | 3.1 × 10¹² | 9.67 × 10³ | 3.12 × 10⁵ | 75.0 |
| Source 15Q | 1.4 × 10¹² | 1.82 × 10³ | 1.30 × 10⁵ | 2.4 |
| Load | | | | |
| Post Source | 6.9 × 10¹² | 7.31 × 10³ | 1.06 × 10⁵ | 1.2 |
| 15Q | | | | |
| Mustang Q | 7.3 × 10¹² | — | — | — |
| Load | | | | |
| Post Mustang | 5.3 × 10¹² | 6.12 | 115.47 | 917.9 |
| Q | | | | |
| Post TFF 2 | 4.9 × 10¹² | — | — | — |
| Final Material | 5.6 × 10¹³ | 8.50 | 15.18 | 7.6 |

### EXAMPLE 11

### Alternative Production Process 70078

The steps in this process were similar to those of process 70065 (Example 10), but had the following changes. The process began with supernatant from two 5 L fermentations; as a general matter, in light of the amount of material, column chromatography was generally performed by splitting the material into two aliquots and performing two column runs.

TFF1 and Benzonase steps: Treatment with Benzonase occurred during the TFF1 filtration step. Specifically, after depth filtration using 4×1.2 μm and 2×0.65 μm Sartopure GF+ filters (Sartorius), 5011.5 g of clarified material was diafiltered for 5 turn-over volumes (TOV), using a 500 kDa MWCO hollow fibre cartridge (0.48 m², 60 cm path length, cat No RTPUFP-500-C-6S) into 25 mM Tris, 100 mM sodium chloride, pH 8.0. The inlet pressure (˜5 psi) was maintained throughout the diafiltration. The Benzonase treatment occurred in the TFF system used for the TFF1 step rather than before the TFF1 step. Specifically, the appropriate volume of benzonase solution to achieve a final concentration of 10 units per mL and 1M MgCl₂ solution to give a final concentration of 5 mM in the diafiltered material were mixed together and injected into the TFF system reservoir bag through the syringe port. The material was then mixed by agitation before being re-circulated in the TFF system at approximately 20% of the running flow rate with the permeate lines closed for 60 minutes at room temperature.

The material was further diafiltered for 5 turn-over volumes (TOV), until the pH and conductivity of the permeate was comparable to the diafiltration buffer (25 mM Tris, 100 mM NaCl, pH 8.0). The inlet pressure (˜5 psi) was maintained throughout the diafiltration.

The recovered retentate (5036.7 g) was 0.8/0.2 μm filtered using Sartopore 2 XLG MidiCap filters (3 filters used to give 4600.8 g (cat no 5445307G9-00)) and sampled for analysis with the remaining bulk stored at 2-8° C.

DEAE, Source 15Q, and Mustang Q steps were performed.

TFF 2 step: The post Mustang Q pool (646.7 g) was concentrated to ˜1.5×10¹⁴ particles/mL based on UV analysis (˜70 mL) using a 500 kDa MWCO hollow fibre cartridge (0.014 m², 30 cm path length (UFP-500C-3MA)).

The material was then buffer exchanged for 5 turn over volumes (TOV) into formulation buffer (1.06 mM potassium phosphate, 2.97 mM sodium phosphate, 155.17 mM sodium chloride, pH 7.4). The shear rate was maintained between 6500 and 8000 sec⁻¹ throughout processing. The retentate was recovered from the system to give 75.6 g.

The material was sampled and then 0.45/0.2 μm filtered using a sterile Sartopore 2 150 filter (Cat no 5441307H4-00-B), The material was then diluted with formulation buffer to target a titre of 1.05×10¹⁴ particles/mL based on UV analysis. Following dilution 74.79 g of final material was generated at a concentration of 9.24×10¹³ virions/mL by UV analysis. The final material had 58.2 EU per 10¹⁴ phage particles (i.e., less than 10⁻¹² EU per phage particle).

Data collected during the process are shown in the following table.

TABLE 30
Endotoxin data from the purification using process 70078.

| | Concentration | Endotoxin | | | | |
|---|---|---|---|---|---|---|
| | by ELISA | | EU/1 × 10¹⁴ | Fold | Total EU | Log |
| Sample | (phage/mL) | EU/mL | particles | Reduction | Load | Reduction |
| Post HIC | 4.09 × 10¹² | 6.96 × 10⁵ | 1.70 × 10⁷ | — | 1.36 × 10⁹ | — |
| Pool | | | | | | |
| Post DEAE | 2.65 × 10¹² | 1.21 × 10⁴ | 4.57 × 10⁵ | 37.2 | 2.98 × 10⁷ | 1.66 |
| Pool | | | | | | |
| Source | 1.70 × 10¹² | 3.94 × 10³ | 2.32 × 10⁵ | 2.0 | — | — |
| 15Q Load | | | | | | |
| Cycle 2 | | | | | | |
| Post | 1.70 × 10¹³ | 2.11 × 10⁴ | 1.24 × 10⁵ | 1.9 | 9.97 × 10⁶ | 0.47 |
| Source | | | | | | |
| 15Q Pool | | | | | | |
| Post | 1.10 × 10¹³ | 2.40 × 10³ | 2.18 × 10⁴ | 5.7 | 1.56 × 10⁶ | 0.81 |
| Mustang Q | | | | | | |
| Final | 6.60 × 10¹³ | 38.4 | 58.2 | 374.7 | 2872 | 2.73 |
| Material | | | | | | |

### EXAMPLE 12

### Processes 70101 and 70107

The steps in these process were similar to those of process 70078 (Example 11), including depth filtration, Benzonase treatment during the TFF1 step, a sequence of chromatography (HIC, DEAF, 15 Q), then Mustang Q filtration and a TFF2 step.

Data collected during the processes are shown in the following tables.

TABLE 31
Endotoxin data from the purification using process 70101.

| | Concentration | Endotoxin | | | | |
|---|---|---|---|---|---|---|
| | by ELISA | | EU/1 × 10¹⁴ | Fold | Total EU | Log |
| Sample | (phage/mL) | EU/mL | particles | Reduction | Load | Reduction |
| Post HIC | 6.7 × 10¹² | 2045.9 | 2.9 × 105 | 4.33 × 10⁶ | 5.93 × 10⁸ | — |
| Pool | | | | | | |
| Post DEAE | 2.1 × 10¹² | 2305.6 | 6380 | 3.04 × 10⁵ | 1.47 × 10⁷ | 1.61 |
| Pool | | | | | | |
| Source | 1.3 × 10¹² | 2242.3 | 3020 | 2.32 × 10⁵ | — | — |
| 15Q Load | | | | | | |
| Cycle 2 | | | | | | |
| Post | 9.6 × 10¹² | 552.8 | 9190 | 9.57 × 10⁴ | 5.08 × 10⁶ | 0.46 |
| Source | | | | | | |
| 15Q Pool | | | | | | |
| Post | 7.2 × 10¹² | 694.2 | 351 | 4875 | 2.44 × 10⁵ | 1.32 |
| Mustang Q | | | | | | |
| Final | 4.7 × 10¹³ | 52.16 | 211 | 449 | 1.10 × 10⁴ | 1.35 |
| Material | | | | | | |

TABLE 32
Endotoxin data from the purification using process 70107.

| | Concentration | Endotoxin | | | | |
|---|---|---|---|---|---|---|
| | by ELISA | | EU/1 × 10¹⁴ | Fold | Total EU | Log |
| Sample | (phage/mL) | EU/mL | particles | Reduction | Load | Reduction |
| Post HIC | 6.8 × 10¹² | 1852.1 | 7.98 × 10⁵ | 1.17 × 10⁷ | 1.48 × 10⁹ | — |
| Pool | | | | | | |
| Post DEAE | 3.4 × 10¹² | 2007.9 | 2.46 × 10⁴ | 7.24 × 10⁵ | 4.94 × 10⁷ | 1.48 |
| Pool | | | | | | |
| Source | 1.8 × 10¹² | 1996.8 | 1.15 × 10⁴ | 6.39 × 10⁵ | 2.30 × 10⁷ | — |
| 15Q Load | | | | | | |
| Cycle 2 | | | | | | |
| Post | 9.3 × 10¹² | 691.0 | 1.93 × 10⁴ | 2.08 × 10⁵ | 1.33 × 10⁷ | 0.57 |
| Source | | | | | | |
| 15Q Pool | | | | | | |
| Post | 6.4 × 10¹² | 874.7 | 8.95 × 10³ | 1.40 × 10⁵ | 7.83 × 10⁶ | 0.23 |
| Mustang Q | | | | | | |
| Final | 7.9 × 10¹³ | 56.6 | 2.71 × 10³ | 3430 | 1.53 × 10⁵ | 1.71 |
| Material | | | | | | |

Process 70107 was run later in time than the other processes in Examples 11 and 12, with much of the same equipment. The overall lower endotoxin reduction across process 70107 in comparison to the previous processes suggested that reuse of the columns may have impacted the contaminant removal efficiency.

### EXAMPLE 13

### Screening of Detergents for Use in Purification Processes

The following detergents were added to TFF1 buffer (25 mM Tris, 100 mM NaCl, pH 8.0) at 1% (w/v) concentration and analysed for interference in the endotoxin assay described above (QCSOP296) by preparing mock samples mimicking dilutions of a TFF1 sample containing 1×10⁵ EU/mL endotoxin:

1. Zwittergent 3-12

2. Zwittergent 3-14

3. Triton X-100

4. Triton X-114

5. Tween 20

The detergents were initially prepared as 5% (w/v) in TFF1 buffer and then diluted to 1% (w/v) in TFF1 buffer to mirror actual process steps. Interference in the Endotoxin assay was measured by the Positive Product Control (PPC) recovery of spiked-in Endotoxin added to each sample. No interference effect was observed, in that %PPC values were within an acceptable range (between 50% and 200% was considered acceptable; values were in the range of 83-117%; data not shown).

A partially processed phage preparation was provided which had been taken through the TFF 1 step in the order of Example 10 (“post TFF 1 material”). The detergents listed in the previous paragraph were added to the post TFF 1 material at two different final concentrations as detailed in Table 33. A run was also performed in the absence of detergent as a control (Run 1). The material was incubated at room temperature for 1 hr with continuous gentle mixing on a roller mixer. Eleven columns of approximately 30 mL Sepharose 6 Fast Flow (XK16 columns) with a 15-17 cm bed height were packed as per the manufacturer's instructions. Each column was sanitised, equilibrated and loaded to ˜20% of the column volume to run as a group separation. The detergents were observed to interfere with chromatographic profiles to varying degrees due to their absorbance at 280 nm.

TABLE 33
Columns Run with Various Detergents

| SEC | | | Volume Post | Volume 5% | VolumeSEC |
|---|---|---|---|---|---|
| Run | | % | TFF 1 | Detergent | Buffer |
| Num- | | Deter- | Material | Stock added | Added |
| ber | Detergent | gent | (mL) | (mL) | (mL) |
| 1 | None | None | 8.0 | 0.0 | 2.0 |
| 2 | Tween 20 | 0.1% | 8.0 | 0.2 | 1.8 |
| 3 | Tween 20 | 1% | 8.0 | 2.0 | 0.0 |
| 4 | Triton X-100 | 0.1% | 8.0 | 0.2 | 1.8 |
| 5 | Triton X-100 | 1% | 8.0 | 2.0 | 0.0 |
| 6 | Triton X-114 | 0.1% | 8.0 | 0.2 | 1.8 |
| 7 | Triton X-114 | 1% | 8.0 | 2.0 | 0.0 |
| 8 | Zwittergent | 0.1% | 8.0 | 0.2 | 1.8 |
| | Z3-12 | | | | |
| 9 | Zwittergent | 1% | 8.0 | 2.0 | 0.0 |
| | Z3-12 | | | | |
| 10 | Zwittergent | 0.1% | 8.0 | 0.2 | 1.8 |
| | Z3-14 | | | | |
| 11 | Zwittergent | 1% | 8.0 | 2.0 | 0.0 |
| | Z3-14 | | | | |

The endotoxin levels and titre as determined by ELISA for the post SEC material from runs 1-11 are shown in Table 34. The 0.1% Triton X-100 (Run 2) and 1% Zwittergent Z3-12 (Run 9) were shown to give the most significant reduction in endotoxin levels. No significant endotoxin removal was observed for the control and other detergents.

TABLE 34
Results for SEC Runs 1-11

| | | | | | Titre by | |
|---|---|---|---|---|---|---|
| | Volume | Result | | EU Log | ELISA | Total |
| Sample | (mL) | (EU/mL) | Total EU | Reduction | (particle/mL) | Particles |
| Post TFF1 | N/A | 1.08 × 10⁷ | — | — | 4.3 × 10¹² | — |
| SEC Load | 6.00 | 8.64 × 10⁶ | 5.18 × 10⁷ | — | 3.4 × 10¹² | 2.04 × 10¹³ |
| Post SEC | 9.99 | 3.52 × 10⁶ | 3.52 × 10⁷ | 0.17 | 2.2 × 10¹² | 2.20 × 10¹³ |
| Control | | | | | | |
| Run 1 | | | | | | |
| Post SEC | 7.91 | 7.86 × 10⁶ | 6.22 × 10⁷ | 0.00 | 2.8 × 10¹² | 2.21 × 10¹³ |
| Run 2 - 0.1% | | | | | | |
| Tween 20 | | | | | | |
| Post SEC | 9.95 | 5.91 × 10⁶ | 5.88 × 10⁷ | 0.00 | 2.5 × 10¹² | 2.49 × 10¹³ |
| Run 3 - 1.0% | | | | | | |
| Tween 20 | | | | | | |
| Post SEC | 9.66 | 1.73 × 10⁵ | 1.67 × 10⁶ | 1.49 | 2.7 × 10¹² | 2.61 × 10¹³ |
| Run 4 A2 - 0.1% | | | | | | |
| Triton X-100 | | | | | | |
| Post SEC | 22.95 | 2.24 × 10⁶ | 5.14 × 10⁷ | — | 1.3 × 10¹⁰ | 2.98 × 10¹¹ |
| Run 4 A3 - 0.1% | | | | | | |
| Triton X-1004 | | | | | | |
| Post SEC | 30.26 | 5.57 × 10⁵ | 1.69 × 10⁷ | 0.49 | 7.6 × 10¹¹ | 2.30 × 10¹³ |
| Run 5 A2 - 1.0% | | | | | | |
| Triton X-100 | | | | | | |
| Post SEC | 20.32 | 4.41 × 10⁶ | 8.96 × 10⁷ | 0.00 | 1.2 × 10¹² | 2.44 × 10¹³ |
| Run 6 A2 - 0.1% | | | | | | |
| Triton X-114 | | | | | | |
| Post SEC | 11.78 | 9.95 × 10⁴ | 1.17 × 10⁶ | — | 2.4 × 10⁹ | 2.83 × 10¹⁰ |
| Run 6 A3 - 0.1% | | | | | | |
| Triton X-1144 | | | | | | |
| Post SEC | 23.75 | 2.33 × 10⁶ | 5.53 × 10⁷ | 0.00 | 9.3 × 10¹¹ | 2.21 × 10¹³ |
| Run 7 - 1.0% | | | | | | |
| Triton X-114 | | | | | | |
| Post SEC | 9.94 | 1.60 × 10⁶ | 1.59 × 10⁷ | 0.51 | 2.2 × 10¹² | 2.19 × 10¹³ |
| Run 8 - 0.1% | | | | | | |
| Zwittergent | | | | | | |
| Z3-12 | | | | | | |
| Post SEC | 9.17 | 3.87 × 10⁵ | 3.55 × 10⁶ | 1.16 | 2.3 × 10¹² | 2.11 × 10¹³ |
| Run 9 - 1.0% | | | | | | |
| Zwittergent | | | | | | |
| Z3-12 | | | | | | |
| Post SEC | 11.04 | 3.12 × 10⁶ | 3.44 × 10⁷ | 0.18 | 2.4 × 10¹² | 2.65 × 10¹³ |
| Run 10 - 0.1% | | | | | | |
| Zwittergent | | | | | | |
| Z3-14 | | | | | | |
| Post SEC | 9.02 | 1.39 × 10⁶ | 1.25 × 10⁷ | 0.62 | 2.7 × 10¹² | 2.44 × 10¹³ |
| Run 11 - 1.0% | | | | | | |
| Zwittergent | | | | | | |
| Z3-14 | | | | | | |

Note:
the EU/mL value for the SEC Load is a theoretical result calculated using the post TFF 1 result and dividing by the 1.25 dilution factor performed during the detergent or buffer (control) addition.

### EXAMPLE 14

### Assessment of Use of Detergents in Column Chromatography Steps

A partially processed phage preparation was provided which had been taken through the TFF1 step in the order of Example 10, and HIC (Toyopearl Phenyl 650M) and DEAE (Fractogel EMD DEAE (M)) steps were performed in the presence of detergent.

In detail, a 21.3 mL HIC column (10.6 cm bed height) and a 21.1 mL DEAE column (10.5 cm bed height) were packed as per the manufacturer's instructions. The columns were re-used for the 4 runs with a cleaning-in-place (CIP) method performed between each run.

Post TFF 1 material was adjusted to the required detergent concentration, or diluted with the corresponding buffer without detergent for the control runs. The material was mixed gently for one hour at room temperature using a magnetic stirrer bar and platform (HIC runs 1-4) or using a roller mixer (DEAE Runs 1-4). The material was then adjusted to the required level of sodium chloride (Table 8) and 0.8/0.2 μm filtered before being immediately loaded onto the respective column. The HIC column was loaded at 5.5×10¹² particles/mL resin based on the theoretical titre as calculated using the Post TFF 1 titre (ELISA) and taking into account the total 2.5× dilution factors applied through adjustment of the material. The reductive DEAE column was loaded at 0.81 mL/mL resin which equates to 0.5 mL Post TFF 1 material/mL resin when taking into account the total 1.61× dilution factor applied through adjustment of the material. The phage material was collected as a single peak for the HIC runs and as 2 mL fractions for the DEAE runs. A small proportion of the reductive DEAE fractions were combined to generate a pool sample for subsequent endotoxin analysis. Endotoxin levels were measured in the samples and the results from the HIC and DEAE runs are shown in Table 35.

TABLE 35
Endotoxin reduction in HIC steps using detergent.

| | | | Endotoxin | | Log |
|---|---|---|---|---|---|
| Sample | Quantity | | (EU/mL) | Total EU | Reduction |
| Post TFF 1 | n/a | | 1.08 × 10⁷ | n/a | — |
| Theoretical HIC | 68.1 | mL | 4.32 × 10⁶ | 2.94 × 10⁸ | — |
| Load Runs 1-4 | | | | | |
| HIC Run 1 | 68.1 | mL | 1.89 × 10⁶ | 1.29 × 10⁸ | — |
| (control) Load | | | | | |
| HIC Run 1 | 18.13 | g | 9.41 × 10⁶ | 1.71 × 10⁸ | 0.2 |
| (control) Pool | | | | | |
| HIC Run 2 (0.1% | 68.1 | mL | 3.76 × 10⁶ | 2.56 × 10⁸ | — |
| Triton) Load | | | | | |
| HIC Run 2 (0.1% | 24.68 | g | 7.01 × 105 | 1.73 × 10⁷ | 1.2 |
| Triton) Pool | | | | | |
| HIC Run 3 (1% | 68.1 | mL | 3.42 × 10⁶ | 2.33 × 10⁸ | — |
| Triton) Load | | | | | |
| HIC Run 3 (1% | 19.29 | g | >5 × 10⁶ | >9.65 × 10⁷ | <0.5 |
| Triton) Pool | | | | | |
| HIC Run 4 (1% | 68.1 | mL | 1.22 × 10⁷ | 8.30 × 10⁸ | — |
| Zwittergent) | | | | | |
| Load | | | | | |
| HIC Run 4 (1% | 13.72 | g | 4.31 × 10⁴ | 5.91 × 10⁵ | 2.7 |
| Zwittergent) | | | | | |
| Pool | | | | | |

Note:
Log Reduction was calculated using the theoretical HIC endotoxin loading.

TABLE 36
Endotoxin reduction in DEAE steps using detergent.

| | Volume | Endotoxin | Total | Log |
|---|---|---|---|---|
| Sample | (mL) | (EU/mL) | Endotoxin | Reduction |
| Post TFF 1 | n/a | 1.08 × 10⁷ | n/a | — |
| Theoretical DEAE | 17.0 | 6.71 × 10⁶ | 1.14 × 10⁸ | — |
| Load Runs 1-4 | | | | |
| DEAE Run 1 | 17.0 | 3.37 × 10⁶ | 5.73 × 10⁷ | — |
| (Control) Load | | | | |
| DEAE Run 1 | 2.0 | 5.88 × 10⁵ | 1.18 × 10⁶ | — |
| (Control) | | | | |
| Fraction A2 | | | | |
| DEAE Run 1 | 2.0 | 1.10 × 10⁶ | 2.20 × 10⁶ | — |
| (Control) | | | | |
| Fraction A5 | | | | |
| DEAE Run 1 | 2.0 | 1.08 × 10⁶ | 2.16 × 10⁶ | — |
| (Control) | | | | |
| Fraction A8 | | | | |
| DEAE Run 1 | 18.0 | 8.39 × 10⁵ | 1.51 × 10⁷ | 0.88 |
| (Control) Pool | | | | |
| (Fractions A1-A9) | | | | |
| DEAE Run 2(0.1% | 17.0 | 1.05 × 10⁷ | 1.79 × 10⁸ | — |
| Triton) Load | | | | |
| DEAE Run 2 0.1% | 2.0 | <5.0 × 10³ | <1 × 10⁴ | — |
| Triton) | | | | |
| Fraction A2 | | | | |
| DEAE Run 2 (0.1% | 2.0 | <5.0 × 10³ | <1 × 10⁴ | — |
| Triton) | | | | |
| Fraction A5 | | | | |
| DEAE Run 2 (0.1% | 2.0 | <5.0 × 10³ | <1 × 10⁴ | — |
| Triton) | | | | |
| Fraction A8 | | | | |
| DEAE Run 2 (0.1% | 18.0 | 357 | 6426 | 4.25 |
| Triton) Pool | | | | |
| (Fractions A1-A9) | | | | |
| DEAE Run 3 (1% | 17.0 | 4.58 × 10⁶ | 7.79 × 10⁷ | — |
| Triton) Load | | | | |
| DEAE Run 3 (1% | 2.0 | 1.89 × 10⁵ | 3.78 × 10⁵ | — |
| Triton) | | | | |
| Fraction A2 | | | | |
| DEAE Run 3 (1% | 2.0 | 3.98 × 10⁶ | 7.96 × 10⁶ | — |
| Triton) | | | | |
| Fraction A5 | | | | |
| DEAE Run 3 (1% | 2.0 | >5 × 10⁶ | >1 × 10⁷ | — |
| Triton) | | | | |
| Fraction A8 | | | | |
| DEAE Run 3 (1% | 24.0 | >5 × 10⁶ | >1.20 × 10⁸ | 0.00 |
| Triton) Pool | | | | |
| (Fractions A1-A12) | | | | |
| DEAE Run 4 (1% | 17.0 | >5 × 10⁶ | >8.50 × 10⁷ | — |
| Zwittergent) Load | | | | |
| DEAE Run 4 (1% | 2.0 | 1.36 × 10⁵ | 2.72 × 10⁵ | — |
| Zwittergent) | | | | |
| Fraction A2 | | | | |
| DEAE Run 4(1% | 2.0 | 2.89 × 10⁴ | 5.78 × 10⁴ | — |
| Zwittergent) | | | | |
| Fraction A5 | | | | |
| DEAE Run 4 1% | 2.0 | 2.79 × 10⁵ | 5.58 × 10⁵ | — |
| Zwittergent) | | | | |
| Fraction A8 | | | | |
| DEAE Run 4(1% | 2.0 | >5 × 10⁶ | >1 × 10⁷ | — |
| Zwittergent) | | | | |
| Fraction A11 | | | | |
| DEAE Run 4(1% | 16.0 | 1.28 × 10⁵ | 2.05 × 10⁶ | 1.6 |
| Zwittergent) Pool | | | | |
| (Fractions A1-A8) | | | | |

Note:
Log Reduction was calculated using the theoretical HIC endotoxin loading.

The reductive DEAE step containing 0.1% Triton was shown to be most effective for endotoxin removal with a 4.4 fold reduction compared to the control where only a 0.5 log was observed. Run 4 containing 1% Zwittergent 3-12 demonstrated a 1.6 log reduction in endotoxin levels when a proportion of the flow though material was pooled (fractions A1-A8). However a later fraction (A11) of the flow through material was shown to contain higher levels of endotoxin.

Based on the above results, the use of Fractogel EMD DEAE (M) with buffer containing 0.1% Triton X-100 was investigated further (see below).

### EXAMPLE 15

### Characterization of DEAE Capacity to Bind Endotoxin

A new 10.9 mL DEAE column (13.9 cm bed height) was packed as per the manufacturer's instructions. A partially processed phage preparation was provided which had been taken through the TFF1 step in the order of Example 11.

This material was adjusted to a final concentration of 0.1% Triton X-100 and incubated for one hour at room temperature, with gentle mixing using a magnetic stirrer bar and platform. The sodium chloride concentration was adjusted to 300 mM and the material 0.8/0.2 μm filtered before being immediately loaded onto the column. The reductive DEAE column was loaded at 0.81 mL/mL resin which equates to 5 mL Post TFF 1 material/mL resin when taking into account the total 1.61× dilution factor applied through adjustment of the material. The chromatography run was performed as per stage 4a DEAE runs 1-4. Fractions were collected throughout the run at 3 mL intervals. Selected fractions were submitted for endotoxin analysis; the results are shown in Table 37.

TABLE 37
Endotoxin content of Fractions From Reductive DEAE Column

| | Column Loading | |
|---|---|---|
| | (mL Post TFF | Endotoxin |
| Fraction | 1/mL media) | (EU/mL) |
| A2 | 0.26 | 7.20E+03 |
| A3 | 0.43 | 8.53E+03 |
| A4 | 0.60 | 1.04E+04 |
| A5 | 0.77 | 2.07E+04 |
| A6 | 0.94 | 2.33E+05 |
| A7 | 1.11 | 2.27E+07 |
| A8 | 1.28 | 3.45E+07 |
| A12 | 1.62 | 8.51E+06 |
| A12 | 1.96 | 2.09E+06 |
| B10 | 2.48 | 2.35E+06 |
| B7 | 2.99 | 2.41E+06 |
| B4 | 3.50 | 3.43E+06 |
| B1 | 4.02 | 1.65E+06 |
| C3 | 4.53 | 5.27E+05 |
| C5 | 4.87 | 2.06E+06 |

The levels of endotoxin were observed to significantly increase above those observed at the start of column loading after fraction A5, corresponding to a loading of 0.77 mL post TFF 1 material/mL media. Based on this result, it was expected that a loading capacity of 80% (i.e., 0.6 mL of Post TFF 1 material/mL media) or less would provide consistent and optimal reduction in endotoxin levels across this step for the reductive DEAE column.

### EXAMPLE 16

### Studies on Alternative Chromatographic Resins

A partially processed phage preparation was provided which had been taken through the TFF1 step in the order of Example 10. This material was diluted with an equal volume of 4M NaCl buffer followed by 0.8/0.2 μm filtration. An HIC Toyopearl Phenyl 650M Column (446 mL CV, 22.8 cm bed height in an XK50 column, new resin) was sanitised and equilibrated prior to use. The column was loaded at 5.5×10¹² phage/mL resin based on a theoretical titre calculated using the post TFF1 titre (by ELISA) and taking into account the 1 in 2 dilution performed, assuming no loss on the filtration step. The column was run as follows:

Flow rate: 97.5 cm/hr (steps other than sample load)

Sample load flow rate: 48.7 cm/hr

Column Equilibration—25 mM Tris, 2M NaCl, pH 7.4

3CV Wash—25 mM Tris, 2 M NaCl, pH 7.4

3CV Elution—25 mM Tris, 250 mM NaCl, pH 7.4

Post HIC material was diluted with 5 volumes of DEAE dilution buffer followed by 0.45/0.2 μm filtration. A binding DEAE (Fractogel EMD DEAE) Column (421.4 mL CV, 21.5 cm bed height in an XK 50 column, new resin) was sanitised and equilibrated prior to use. The column was loaded at 5×10¹² phage/mL resin based on a theoretical DEAE load titre calculated using the post HIC titre (by OD) and taking into account the 1 in 6 dilution performed, assuming no loss on the filtration step. The column was run as follows:

Flow rate: 97.46 cm/hr (all steps)

Column Equilibration—25 mM phosphate, 100 mM NaCl, pH 6.5

2CV Wash—25 mM phosphate, 150 mM NaCl, pH 6.5

4CV Wash—25 mM phosphate, 250 mM NaCl, pH 6.5

3CV Elution—25 mM phosphate, 300 mM NaCl, pH 6.5

The post binding DEAE material thus produced was used to assess the efficacy of possible subsequent steps for further reduction of endotoxin levels. Unless otherwise indicated, for the columns described in the following paragraphs, the phage flow through peak was collected as 5 mL fractions when A256 started at and dropped down to 5% of the peak maximum.

Post binding DEAE material was loaded onto a reductive DEAE column based on a loading of 4 mL/mL resin. The reductive DEAE column (17.08 mL column volume, Fractogel EMD DEAE, 8.5 cm bed in an XK 16 column, new resin) was sanitised and equilibrated prior to use.

Post binding DEAE material was loaded onto a reductive Q Sepharose XL column based on a loading of 4 mL/mL resin. The reductive QXL column (14.67 mL column volume, 7.3 cm bed height in an XK 16 column, new resin) was sanitised and equilibrated prior to use.

5 mL pre-packed EtoxiClear columns (ProMetic BioSciences Ltd., Rockville, Md.) were sanitised and equilibrated in the appropriate buffer prior to use. A new EtoxiClear column was used for runs 1, 2 and 3 and the column used for run 1 was re-used for both runs 4 and 5 with a sanitisation step performed between runs. Post binding DEAE material was loaded without dilution for runs 1 and 4, diluted to give a final NaCl concentration of 200 mM NaCl for runs 2 and 5, and diluted to give a final NaCl concentration of 100 mM for run 3. Runs 4-5, performed at pH 5.0, utilised post binding DEAE material that had been buffer exchanged via dialysis to reduce pH using snakeskin tubing (10 kDa MWCO) at 2-8° C.

The columns were loaded at ˜40 mL post binding DEAE material/mL resin (see Table 38). The endotoxin loading was subsequently determined as 32,800 EU/mL resin and 27,200 EU/mL resin for runs 1 and 2 respectively and ˜15,000 EU/mL resin for runs 4 and 5. The flow through unbound material was washed out with the appropriate equilibration buffer. The phage peak was collected as multiple fractions when A₂₅₆ started at and dropped down to 20 mAU. The fraction size was adjusted to account for the dilution in the load material (Table 38). The column load and run in 100 mM NaCl (Run 3) showed partial binding of the phage material, which was eluted from the column using 25 mM phosphate, 300 mM NaCl, pH 6.5.

TABLE 38
EtoxiClear Run Conditions.

| | | | Volume | Volume | |
|---|---|---|---|---|---|
| | | | Dilution | Loaded | Volume |
| | Condition | Volume | Buffer | onto | Fractions |
| Etoxiclear | (Equilibration | Sample | Added | Column | Collected |
| Run | buffer) | (mL) | (mL) | (mL) | (mL) |
| Run 1 | 300 mM NaCl, | 40 | 0 | 40 | 2.5 |
| | 25 mM Phosphate, | | | | |
| | pH 6.5 | | | | |
| Run 2 | 200 mM NaCl, | 40 | 20 | 58 | 3.75 |
| | 25 mM Phosphate, | | | | |
| | pH 6.5 | | | | |
| Run 3 | 100 mM NaCl, | 40 | 80 | 118 | 7.5 |
| | 25 mM Phosphate, | | | | |
| | pH 6.5 | | | | |
| Run 4 | 300 mM NaCl, | 40 | 0 | 40 | 2.5 |
| | 50 mM Acetate, | | | | |
| | pH 5.0 | | | | |
| Run 5 | 200 mM NaCl, | 40 | 20 | 58 | 3.75 |
| | 50 mM Acetate, | | | | |
| | pH 5.0 | | | | |

The Endotoxin results for the reductive anion exchange (AEX) and EtoxiClear runs are shown in Table 39. As the capacity for both the reductive AEX and EtoxiClear for endotoxin was initially unknown, the flow through fractions were not pooled but selected fractions analysed separately for endotoxin and titre by OD to evaluate the performance of the column steps.

The reductive AEX (DEAE and QXL) showed less than 1 log reduction in endotoxin when comparing the endotoxin levels (EU/mL) in the load material to the flow through fractions analysed.

The EtoxiClear chromatography performed in the presence of 200-300 mM NaCl (Runs 1, 2, 3 and 4) demonstrated an approximate 3.4-4.9 log reduction in endotoxin comparing endotoxin levels (EU/mL) in the load material to the flow through fractions analysed. The titre measurements indicated that there was no significant loss in yield over the EtoxiClear step for runs 1, 2, 4 and 5.

Thus, the screen of the EtoxiClear resin demonstrated promising results for significant reductions in endotoxin levels and was selected for further investigation. It was noted that performing the EtoxiClear chromatography in 25 mM phosphate, 300 mM NaCl, pH 6.5 could be carried out following the DEAE chromatography step without an additional buffer exchange step.

TABLE 39
Endotoxin Results for Reductive AEX and EtoxiClear Steps

| | | | Result | Estimate Log | Titre by OD |
|---|---|---|---|---|---|
| Sample | Volume | | EU/mL | Reductions | (particles/mL) |
| Post HIC Pool | 452.46 | g | 1.22 × 10⁶ | — | 7.00 × 10¹² |
| Binding DEAE | 1747 | mL | 1.27 × 10⁵ | — | 1.21 × 10¹² |
| Load | | | | | |
| Post Binding | 421.7 | g | 4.20 × 10³ | — | 3.08 × 10¹² |
| DEAE Material | | | | | |
| Reductive | 79.0 | g | 4.73 × 10³ | — | 3.57 × 10¹² |
| DEAE Load | | | | | |
| Run 1 | | | | | |
| Post Reductive | 5 | mL | 2.23 × 10³ | 0.3 | 2.95 × 10¹² |
| DEAE Fraction | | | | | |
| A4 | | | | | |
| Post Reductive | 5 | mL | 1.83 × 10³ | 0.3 | 2.98 × 10¹² |
| DEAE Fraction | | | | | |
| A6 | | | | | |
| Post Reductive | 5 | mL | 2.20 × 10³ | 0.3 | 3.02 × 10¹² |
| DEAE Fraction | | | | | |
| A8 | | | | | |
| Reductive QXL | 79.8 | g | 4.73 × 10³ | — | 3.57 × 10¹² |
| Load Run 2 | | | | | |
| Post Reductive | 5 | mL | 1.80 × 10³ | 0.4 | 2.98 × 10¹² |
| QXL Fraction | | | | | |
| A4 | | | | | |
| Post Reductive | 5 | mL | 2.21 × 10³ | 0.3 | 2.97 × 10¹² |
| QXL Fraction | | | | | |
| A6 | | | | | |
| Post Reductive | 5 | mL | 2.14 × 10³ | 0.3 | 3.00 × 10¹² |
| QXL Fraction | | | | | |
| A8 | | | | | |
| EtoxiClear | 40 | mL | 4.10 × 10³ | — | 3.85 × 10¹² |
| Run 1 Load | | | | | |
| EtoxiClear | 2.5 | mL | 0.0738 | 4.7 | 2.60 × 10¹² |
| Pool Run 1 | | | | | |
| A5 | | | | | |
| EtoxiClear | 2.5 | mL | 0.108 | 4.6 | 2.94 × 10¹² |
| Pool Run 1 | | | | | |
| A8 | | | | | |
| EtoxiClear | 2.5 | mL | <0.05 | >4.9 | 3.03 × 10¹² |
| Pool Run 1 | | | | | |
| A11 | | | | | |
| EtoxiClear | 58 | mL | 2.34 × 10³ | — | 2.93 × 10¹² |
| Run 2 Load | | | | | |
| EtoxiClear | 3.75 | mL | <0.05 | >4.7 | 1.89 × 10¹² |
| Pool Run 2 | | | | | |
| A5 | | | | | |
| EtoxiClear | 3.75 | mL | <0.05 | >4.7 | 1.99 × 10¹² |
| Pool Run 2 | | | | | |
| A8 | | | | | |
| EtoxiClear | 3.75 | mL | 0.0718 | 4.5 | 1.99 × 10¹² |
| Pool Run 2 | | | | | |
| A11 | | | | | |
| EtoxiClear | 5.28 | g | 4.59 | — | 1.31 × 10¹² |
| Pool Run 3 | | | | | |
| Eluted | | | | | |
| EtoxiClear | 7.5 | mL | 19.2 | — | 6.04 × 10¹² |
| Pool Run 3 | | | | | |
| B11 | | | | | |
| EtoxiClear | 40 | mL | 1.89 × 10³ | — | 4.22 × 10¹² |
| Run 4 Load | | | | | |
| EtoxiClear | 2.5 | mL | <0.05 | >4.6 | 2.58 × 10¹² |
| Pool Run 4 | | | | | |
| A6 | | | | | |
| EtoxiClear | 2.5 | mL | 0.0929 | 4.3 | 2.82 × 10¹² |
| Pool Run 4 | | | | | |
| A9 | | | | | |
| EtoxiClear | 2.5 | mL | 0.150 | 4.1 | 2.87 × 10¹² |
| Pool Run 4 | | | | | |
| A12 | | | | | |
| EtoxiClear | 58 | mL | 1.34 × 10³ | — | 3.16 × 10¹² |
| Run 5 Load | | | | | |
| EtoxiClear | 3.75 | mL | 0.286 | 3.7 | 1.71 × 10¹² |
| Pool Run 5 | | | | | |
| A5 | | | | | |
| EtoxiClear | 3.75 | mL | 0.516 | 3.4 | 1.87 × 10¹² |
| Pool Run 5 | | | | | |
| A8 | | | | | |
| EtoxiClear | 3.75 | mL | 0.529 | 3.4 | 1.86 × 10¹² |
| Pool Run 5 | | | | | |
| A11 | | | | | |

### EXAMPLE 17

### Studies on Alternative Combinations of Purification Steps

A partially processed phage preparation (“post-TFF1 material”) was provided which had been taken through the TFF 1 step in the order of Example 11. Three combinations of purification steps were performed and the level of endotoxin removal was evaluated.

In the first combination of steps (Run 1 in Table 40 below), Triton X-100 was added to the post-TFF1 material to a final concentration of 0.1% and NaCl was added to a final concentration of 300 mM. After addition of Triton X-100 and NaCl, the material was incubated for 1 hour followed by 0.45/0.2 μm filtration (2× Sartopore 2 150 filters). Reductive DEAE chromatography was performed using 25 mM Tris, 300 mM NaCl, pH7.4 as the buffer conditions on a Fractogel EMD DEAE column (415 mL column volume (CV), 21.17 cm bed height in a XK50 column (new resin)) which was sanitized and equilibrated prior to use. The post filtered, NaCl and Triton X-100 adjusted material was loaded onto the DEAE column based on a loading of 0.5 mL Post TFF 1 material/mL resin (taking into account the total dilution factor of x1.31 following adjustment to generate the load material). The phage peak was collected as a single pool when A₂₅₄ started at and dropped down to 20mAU. Samples that required storage at ≦−65° C. were snap frozen with liquid nitrogen and stored at ≦−65° C. at the end of the processing day. All other samples and bulk material were held at 2-8° C.

The flowthrough containing phage was diluted with 5 volumes of 25 mM phosphate, pH 6.5 followed by 0.45/0.2 μm filtration (1× Sartopore 2 300 filter). This material was then loaded on a binding DEAE chromatography column (Fractogel EMD DEAE, 229 mL CV, 11.68cm bed height in a XK50 column (new resin)) at 5×10¹² phage/mL resin based on a theoretical titre calculated using the post TFF1 titre as determined by ELISA, taking into account the dilution of material through adjustment and also the increase in volume over the reductive DEAE step and assuming a 90% step yield for the reductive DEAE step. A sample of the DEAE load was taken and analysed retrospectively for titre as determined by ELISA. The binding DEAE column loading was retrospectively determined as 1.6×10¹² particles/mL resin by ELISA. The binding DEAE column was washed with buffer containing 250 mM NaCl and eluted with 25 mM phosphate, 300 mM NaCl, pH 6.5. The post binding DEAE material was analysed on-line (the same day) for endotoxin and determined to be at 1.17×10³ EU/mL and then passed through a 5 mL new, pre-packed, santised, equilibrated EtoxiClear column (without adjustment of buffer) at 10000 EU/mL resin based on the on-line measurement. A second sample of DEAE Pool material sampled the following day and termed EtoxiClear load was analysed retrospectively for endotoxin as 931 EU/mL giving a column loading of 7960 EU/mL resin. The difference in column loading determination between the two results is likely to be due to the variation of the assay. The phage product was loaded onto the column and collected as the flow through fraction when the A₂₅₄ increased to 20 mAU and dropped back down to 20 mAU following a wash step with equilibration buffer. The fractions collected were pooled at the end of the run. Samples requiring snap freezing were performed at the end of the processing day with liquid nitrogen and stored at ≦−65° C. Remaining samples were held at 2-8° C.

In the second combination of steps (Run 2 in Table 40 below), the post-TFF1 material was diluted 1:1 with 25 mM Tris, 4M NaCl, pH 7.4 followed by 0.45/0.2 μm filtration (1× Sartopore 2 150), and then loaded on a sanitised, equilibrated HIC column (Toyopearl Phenyl 650M, 440 mL CV, 22.4 cm bed height in an XK50 column, resin used for one cycle previously). The material was eluted with 25 mM Tris, 250 mM NaCl, pH 7.4. The phage peak was collected as a single pool when A₂₅₄ started at and dropped down to 20 mAU. It was observed that the phage peak began to elute shortly after the conductivity of the eluate began to drop, resulting in an NaCl concentration greater than 250 mM. Samples requiring snap freezing were performed at the end of the processing day with liquid nitrogen and stored at ≦−65° C. Remaining samples were held at 2-8° C.

Triton X-100 was added to a final concentration of 0.1% and NaCl was added to a calculated final concentration of 300 mM based on the assumption that the eluate from the previous step contained NaCl at 250 mM. The material was then back-diluted 2.5 fold with 25 mM Tris pH 7.4, giving a conductivity matching the column equilibration buffer for the next column (29.1 mS). Additional Triton X-100 was added as well to maintain a 0.1% concentration. This material was incubated for 1 hour followed by 0.45/0.2 μm filtration (1× Sartopore 2 150).

Reductive DEAE chromatography was performed using 25 mM Iris, 300 mM NaCl, pH7.4 as the buffer conditions; a reductive DEAE column (Fractogel EMD DEAE, 80 mL column volume (CV), 15 cm bed height in a XK26 column (new resin)) was loaded at 3.09 mL post-HIC material per mL resin (calculated taking into account the total dilution factor of x2.9 for the dilution and adjustment steps). The flow through phage material was collected as a single pool when A₂₅₄ started at and washed down to 20 mAU with equilibration buffer. Samples requiring snap freezing were performed at the end of the processing day with liquid nitrogen and stored at ≦−65° C. Remaining samples were held at 2-8° C.

The post-reductive DEAE material was diluted with 5 volumes of 25 mM phosphate, pH 6.5, followed by 0.45/0.2 μm filtration (1× Sartopore 2 300). This diluted material was then loaded on a sanitised, equilibrated binding DEAE chromatography column (Fractogel EMD DEAE, 372 mL CV, 19 cm bed height in an XK50 column (new resin)), washed with buffer containing 250 mM NaCl, and eluted with 25 mM phosphate, 300 mM NaCl, pH 6.5. The loading of the binding DEAE step could not be determined by OD due to the presence of Triton-X100 in the load sample and the low concentration at this point. Therefore the column loading was based on a theoretical titre calculated using the titre of the post HIC material as determined by OD and an assumption of a 90% reductive DEAE step yield whilst taking into account the material adjustment/dilution steps and the volume increase over the reductive DEAE step. Using this theoretical titre the column was loaded at 5×10¹² phage/mL resin. The actual binding DEAE column loading was retrospectively determined as 4.4×10¹² particles/mL resin by ELISA. The phage peak was collected as a single pool when A254 started at and dropped down to 20 mAU. Samples requiring snap freezing were performed at the end of the processing day with liquid nitrogen and stored at ≦65° C. Remaining samples were held at 2-8° C.

The post-reductive DEAE material was analysed on-line (the same day) for endotoxin and determined to be at 1.57 EU/mL. As the on-line endotoxin level was determined to be significantly lower than runs 1 and 3, the column could not be loaded at 10000 EU/mL resin. Therefore, all of the available post binding DEAE material was loaded onto the column to give a column loading of 63 EU/mL resin, then passed through a 5 mL pre-packed, sanitised, equilibrated EtoxiClear column (used previously for 1 cycle). Phage product was collected as the flow through fraction when the A254 increased to 20 mAU and dropped back down to 20 mAU following a wash step with equilibration buffer (25 mM phosphate, 300 mM NaCl, pH 6.5).

In the third combination of steps (Run 3 in Table 40 below), post-HIC material generated from Run 2 was used. It was diluted with 5 volumes of dilution buffer followed by 0.45/0.2 μm filtration (1× Sartopore 2 150). Post filtration material was loaded onto the binding DEAE column at 5×10¹² phage/mL resin based on a theoretical binding DEAE load titre calculated using the post HIC pool titre as determined by OD and taking into account the 1 in 6 dilution of the binding DEAE load material, assuming no loss on filtration. The binding DEAE column loading was retrospectively determined as 5.3×10¹² particles/mL resin by ELISA.

The binding DEAE column (Fractogel EMD DEAE, 34 mL CV, 16.9 cm bed height in an XK16 column (new resin)) was sanitised and equilibrated prior to use. The material was loaded onto the column and a wash step performed using wash buffer containing 250 mM NaCl, before the phage was eluted with 300 mM NaCl. The phage peak was collected as a single pool when A254 started at and dropped down to 20 mAU. Samples requiring snap freezing were performed at the end of the processing day with liquid nitrogen and stored at ≦−65° C. Remaining samples were held at 2-8° C.

The post binding DEAE material was analysed on-line (the same day) for endotoxin and determined to be at 1.34 E+4 EU/mL. The column was loaded at 10720 EU/mL resin based on the on-line endotoxin data. A second sample of DEAE Pool material sampled the following day and termed EtoxiClear load was analysed retrospectively for endotoxin as 7.03 E+3 EU/mL giving a column loading of 5624 EU/mL resin.

An EtoxiClear column (5 mL pre-packed new column) was sanitised and equilibrated prior to use. The phage material was loaded onto the column and collected as the flow through fraction when the A₂₅₄ increased to 20 mAU and dropped back down to 20 mAU following a wash step with equilibration buffer (25 mM phosphate, 300 mM NaCl, pH 6.5). Samples requiring snap freezing were performed at the end of the processing day with liquid nitrogen and stored at ≦−65° C. Remaining samples were held at 2-8° C.

General Notes Regarding Runs 1-3 of this Example: Analysis of post EtoxiClear material for the three process runs showed that there was no residual Benzonase detected and the infectivity as determined by plaque assay was comparable between runs (5.1-6.3×10¹¹ pfu/mL), within the error of the assay. There is a known inherent variation for the ELISA assay as the assay is non-specific. The ELISA assay uses a commercial G3 protein capture antibody which actually binds the G8 protein.

Results from Runs 1, 2, and 3 are shown in the following tables.

TABLE 40
Endotoxin Analysis for Runs 1, 2, and 3.

| | Process | | | Endotoxin | | Endotoxin Log | EU/1 × 10¹⁴ |
|---|---|---|---|---|---|---|---|
| Run | Step | Volume | | (EU/mL) | Total EU | Reduction | particles by ELISA |
| 1 | Reductive | 271.8 | mL | 8.50 × 10⁶ | 2.31 × 10⁹ | — | — |
| | DEAE Load | | | | | | |
| 1 | Reductive | 315.1 | g | 3.39 × 10⁴ | 1.07 × 10⁷ | 2.33 | — |
| | DEAE Pool | | | | | | |
| 1 | Binding | 773.4 | mL | 3.36 × 10³ | 2.60 × 10⁶ | — | — |
| | DEAE Load | | | | | | |
| 1 | Binding | 101.0 | g | 1.17 × 10³ | 1.18 × 10⁵ | 1.34 | — |
| | DEAE Pool | | | | | | |
| 1 | EtoxiClear | 42.7 | mL | 931 | 3.98 × 10⁴ | — | — |
| | Load | | | | | | |
| 1 | EtoxiClear | 44.6 | g | 0.63 | 28.1 | 3.15 | 33 |
| | Pool | | | | | | |
| 2 + 3 | HIC Load | 322.7 | mL | 2.60 × 10⁶ | 8.39 × 10⁸ | — | — |
| 2 + 3 | HIC Pool | 303.1 | g | 2.27 × 10⁵ | 6.88 × 10⁷ | 1.09 | — |
| 2 | Reductive | 717.9 | mL | 3.03 × 10⁴ | 2.18 × 10⁷ | — | — |
| | DEAE Load | | | | | | |
| 2 | Reductive | 767.8 | g | 5.29 × 10⁴ | 4.06 × 10⁷ | 0.00 | — |
| | DEAE Pool | | | | | | |
| 2 | Binding | 4319.5 | g | 4.58 × 10⁴ | 1.98 × 10⁸ | — | — |
| | DEAE Load | | | | | | |
| 2 | Binding | 209.4 | g | 1.57 | 328.8 | 5.78 | — |
| | DEAE Pool | | | | | | |
| 2 | EtoxiClear | 200.0 | mL | <5.00 | <1000 | — | — |
| | Load | | | | | | |
| 2 | EtoxiClear | 202.5 | g | <0.013 | <2.03 | >2.703 | <0.53 |
| | Pool | | | <0.01 | | | |
| 3 | Binding | 180.9 | mL | 4.29 × 10⁴ | 7.76 × 10⁶ | — | — |
| | DEAE Load | | | | | | |
| 3 | Binding | 19.3 | g | 1.34 × 10⁴ | 2.59 × 10⁵ | 1.48 | — |
| | DEAE Pool | | | | | | |
| 3 | EtoxiClear | 4.0 | mL | 7.03 × 10³ | 2.81 × 10⁴ | — | — |
| | Load | | | | | | |
| 3 | EtoxiClear | 5.3 | g | <0.013 | <0.053 | >5.73 | <0.53 |
| | Pool | | | | | | |

TABLE 41
Host Cell Protein Analysis for Runs 1, 2, and 3.

| | | | | | Total | | |
|---|---|---|---|---|---|---|---|
| | Process | | | HCP | HCP | HCP Log | HCP/1 × 10¹⁴ |
| Run | Step | Volume | | (ng/mL) | (ng) | Reduction | Particles by ELISA |
| 1 | Reductive | 271.8 | mL | 71286 | 1.94 × 10⁷ | — | — |
| | DEAE Load | | | | | | |
| 1 | Reductive | 315.1 | g | 24302 | 7.66 × 10⁶ | 0.4 | — |
| | DEAE Pool | | | | | | |
| 1 | Binding | 773.4 | mL | 40502 | 3.13 × 10⁶ | — | — |
| | DEAE Load | | | | | | |
| 1 | Binding | 101.0 | g | 6.35 | 641.4 | 3.7 | — |
| | DEAE Pool | | | | | | |
| 1 | EtoxiClear | 42.7 | mL | 6.35 | 271.1 | — | — |
| | Load | | | | | | |
| 1 | EtoxiClear | 44.6 | g | 4.40 | 196.2 | 0.1 | 232 |
| | Pool | | | | | | |
| 2 + 3 | HIC Load | 322.7 | mL | 5565 | 1.80 × 10⁶ | — | — |
| 2 + 3 | HIC Pool | 303.1 | g | 661 | 2.00 × 10⁵ | 1.0 | — |
| 2 | Reductive | 717.9 | mL | 2277 | 1.63 × 10⁵ | — | — |
| | DEAE Load | | | | | | |
| 2 | Reductive | 767.8 | g | 1429 | 1.10 × 10⁶ | 0.0 | — |
| | DEAE Pool | | | | | | |
| 2 | Binding | 4319.5 | g | 2387 | 1.03 × 10⁶ | — | — |
| | DEAE Load | | | | | | |
| 2 | Binding | 209.4 | g | 3.49 | 730.8 | 3.1 | — |
| | DEAE Pool | | | | | | |
| 2 | EtoxiClear | 200.0 | mL | 3.49 | 698.0 | — | — |
| | Load | | | | | | |
| 2 | EtoxiClear | 202.5 | g | 1.81 | 366.5 | 0.3 | 95 |
| | Pool | | | | | | |
| 3 | Binding | 180.9 | mL | 1107 | 1.99 × 10⁴ | — | — |
| | DEAE Load | | | | | | |
| 3 | Binding | 19.3 | g | 19.52 | 376.7 | 1.7 | — |
| | DEAE Pool | | | | | | |
| 3 | EtoxiClear | 4.0 | mL | 19.52 | 78.1 | — | — |
| | Load | | | | | | |
| 3 | EtoxiClear | 5.3 | g | 1.46 | 7.7 | 1.0 | 70 |
| | Pool | | | | | | |

TABLE 42
Titre and Step Yield Data for Runs 1, 2 and 3 (ELISA and OD).

| | | | | Titre by | Total | % Step | Titre by | Total | % Step |
|---|---|---|---|---|---|---|---|---|---|
| | Process | | | ELISA | Particles | Yield by | OD | Particles | Yield |
| Run | Step | Volume | | (particle/mL) | by ELISA | ELISA | (particles/mL) | by OD | by OD |
| 1 | Reductive | 271.8 | mL | 1.5 × 10¹³ | 4.08 × 10¹⁵ | — | Not determined due to | | |
| | DEAE | | | | | | potential Triton interference | | |
| | Load | | | | | | | | |
| 1 | Reductive | 315.1 | g | 3.6 × 10¹² | 1.13 × 10¹⁵ | ~28% | | | |
| | DEAE | | | | | | | | |
| | Pool | | | | | | | | |
| 1 | Binding | 773.4 | mL | 4.6 × 10¹¹ | 3.56 × 10¹⁴ | — | | | |
| | DEAE | | | | | | | | |
| | Load | | | | | | | | |
| 1 | Binding | 101.0 | g | 1.2 × 10¹² | 1.21 × 10¹⁴ | ~34% | | | |
| | DEAE | | | | | | | | |
| | Pool | | | | | | | | |
| 1 | EtoxiClear | 42.7 | mL | 1.3 × 10¹² | 5.55 × 10¹³ | — | | | |
| | Load | | | | | | | | |
| 1 | EtoxiClear | 44.6 | g | 1.9 × 10¹² | 8.47 × 10¹³ | >100% | | | |
| | Pool | | | | | | | | |
| 2 + 3 | HIC Load | 322.7 | mL | 1.5 × 10¹² | 4.84 × 10¹⁴ | — | 6.52 × 10¹² | 2.10 × 10¹⁵ | — |
| 2 + 3 | HIC Pool | 303.1 | g | 4.1 × 10¹² | 1.24 × 10¹⁵ | >100% | 5.59 × 10¹² | 1.69 × 10¹⁵ | ~80% |
| 2 | Reductive | 717.9 | mL | 1.4 × 10¹² | 1.00 × 10¹⁵ | — | Not determined due to | | |
| | DEAE | | | | | | potential Triton interference | | |
| | Load | | | | | | | | |
| 2 | Reductive | 767.8 | g | 6.4 × 10¹¹ | 4.91 × 10¹⁴ | ~49% | | | |
| | DEAE | | | | | | | | |
| | Pool | | | | | | | | |
| 2 | Binding | 4319.5 | g | 3.8 × 10¹¹ | 1.64 × 10¹⁵ | — | | | |
| | DEAE | | | | | | | | |
| | Load | | | | | | | | |
| 2 | Binding | 209.4 | g | 2.6 × 10¹² | 5.44 × 10¹⁴ | ~33% | | | |
| | DEAE | | | | | | | | |
| | Pool | | | | | | | | |
| 2 | EtoxiClear | 200.0 | mL | 3.2 × 10¹² | 6.40 × 10¹⁴ | — | | | |
| | Load | | | | | | | | |
| 2 | EtoxiClear | 202.5 | g | 1.9 × 10¹² | 3.85 × 10¹⁴ | ~60% | | | |
| | Pool | | | | | | | | |
| 3 | Binding | 180.9 | mL | 1.0 × 10¹² | 1.81 × 10¹⁴ | — | 2.45 × 10¹² | 4.43 × 10¹⁴ | — |
| | DEAE | | | | | | | | |
| | Load | | | | | | | | |
| 3 | Binding | 19.3 | g | 2.5 × 10¹² | 4.83 × 10¹³ | ~27% | 6.08 × 10¹² | 1.17 × 10¹⁴ | ~26% |
| | DEAE | | | | | | | | |
| | Pool | | | | | | | | |
| 3 | EtoxiClear | 4.0 | mL | 3.1 × 10¹² | 1.24 × 10¹³ | — | 5.59 × 10¹² | 2.24 × 10¹³ | — |
| | Load | | | | | | | | |
| 3 | EtoxiClear | 5.3 | g | 2.1 × 10¹² | 1.11 × 10¹³ | ~90% | 4.00 × 10¹² | 2.12 × 10¹³ | ~95% |
| | Pool | | | | | | | | |

TABLE 43
Summary of Results of Chromatography Steps in Runs 1, 2, and 3

| Process | Endotoxin Log Reduction | | | HCP Log Reduction | | | Step Yield by ELISA | | |
|---|---|---|---|---|---|---|---|---|---|
| Step | Run 1 | Run 2 | Run 3 | Run 1 | Run 2 | Run 3 | Run 1 | Run 2 | Run 3 |
| HIC | — | 1.09 | As run | — | 1.0 | As run | — | >100% | As run |
| | | | 2 | | | 2 | | | 2 |
| Reductive | 2.33 | 0.00 | — | 0.4 | 0.0 | — | ~28% | ~49% | — |
| DEAE | | | | | | | | | |
| Binding | 1.34 | 5.78 | 1.48 | 3.7 | 3.1 | 1.7 | ~34% | ~33% | ~27% |
| DEAE | | | | | | | | | |
| EtoxiClear | 3.15 | >2.7 | >5.70 | 0.1 | 0.3 | 1.0 | >100% | ~60% | ~90% |

Based on these results, the binding DEAE step was shown to give the greatest reduction in HCP levels, which appeared to be more effective when Triton X-100 was present in the load material for this column (runs 1 and 2 compared to run 3 without detergent). Process runs 2 and 3 with the inclusion of the HIC step were shown to generate post EtoxiClear material with the lowest levels of HCP at 1.5-1.8 ng/mL (Table 41) which standardised to 1×10¹⁴ particles gives 95 and 70 ng/1×10¹⁴ particles for runs 2 and 3 respectively (Table 42). The best performing steps for endotoxin reduction were indicated to be the binding DEAE when performed following a HIC step and with Triton X-100 present in the load material (run 2, 5.78 log reduction (Table 40)) and the EtoxiClear step with 2.7 to 5.7 log reduction (runs 1-3). The post EtoxiClear material from runs 2 and 3 achieved endotoxin levels of <0.01 EU/mL which standardised to 1×10¹⁴ particles gives <0.5 EU/1×10¹⁴ particles.

### EXAMPLE 18

### Additional Purification Protocols

The following protocol for purifying filamentous bacteriophage are also within the methods according to the invention. It is understood that one skilled in the art would carry out filtration of material, and sanitization and equilibration of columns at appropriate times.

According to Run 3b, post-TFF1 material is provided and diluted 1:1 with 25 mM Tris, 4M NaCl, pH 7.4. HIC chromatography is then performed with elution using 25 mM Tris, 250 mM NaCl, pH 7.4. The post-HIC material is then diluted with 5 volumes of 25 mM phosphate, pH 6.5. Next, the diluted material is subjected to binding DEAE chromatography with a wash step followed by elution at 25 mM phosphate, 300 mM NaCl, pH 6.5. The post-binding DEAE material is then passed through an EtoxiClear column, also using 25 mM phosphate, 300 mM NaCl, pH 6.5.

According to Run 4, post-TFF1 material is provided and diluted 1:1 with 25 mM Tris, 4M NaCl, pH 7.4. HIC chromatography is then performed with elution using 25 mM Tris, 250 mM NaCl, pH 7.4. The post-HIC material is then diluted with 5 volumes of 0.12% Triton-X100, 25 mM phosphate, pH 6.5 (such that the diluted material contains 0.1% Triton X-100). Next, the diluted material is subjected to binding DEAE chromatography with a wash step followed by elution at 25 mM phosphate, 300 mM NaCl, pH 6.5. The post-binding DEAE material is then passed through an EtoxiClear column, also using 25 mM phosphate, 300 mM NaCl, pH 6.5.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A pharmaceutical composition comprising wild-type filamentous bacteriophage or filamentous bacteriophage which does not display an antibody or a non-filamentous bacteriophage antigen on its surface, said composition comprising less than 1×10⁻¹⁰ endotoxin units per filamentous bacteriophage; and a pharmaceutically acceptable carrier.

2. The pharmaceutical composition of claim 1, comprising less than 1×10⁻¹¹ endotoxin units per filamentous bacteriophage.

3. The pharmaceutical composition of claim 1, comprising less than 1×10⁻¹² endotoxin units per filamentous bacteriophage.

4. The pharmaceutical composition of claim 1, comprising less than 1×10⁻¹³ endotoxin units per filamentous bacteriophage.

5. The pharmaceutical composition of claim 1, comprising less than 5×10⁻¹⁴ endotoxin units per filamentous bacteriophage.

6. The pharmaceutical composition of claim 1, wherein the composition is a liquid composition.

7. The pharmaceutical composition of claim 1, having at least 4×10¹⁷ filamentous bacteriophage.

8. The pharmaceutical composition of claim 1 wherein the filamentous bacteriophage are M13.

9. The pharmaceutical composition of claim 1 in a solid form.

10. The pharmaceutical composition of claim 9, formulated into tablets, granulates, nano-particles, nano-capsule micro-capsules, micro-tablets, pellets, or powders.

11. The pharmaceutical composition of claim 1 formulated into a single dosage form.

12. The pharmaceutical composition of claim 11, wherein the single dosage form is contained in a vial.

13. The pharmaceutical composition of claim 11, wherein the single dosage form is contained in an infusion bag or pump reservoir.

14. The pharmaceutical composition of claim 11, wherein the single dosage form is contained in one or more tablets or capsules.

15. The pharmaceutical composition of claim 1, comprising an amount of endotoxin that when administered to a human provides less than 5.0 endotoxin units per kilogram body weight per dose.

16. The pharmaceutical composition of claim 15, comprising an amount of endotoxin that when administered to a human provides less than 0.2 endotoxin units per kilogram body weight per dose.

17. A method of reducing the amount of amyloid plaque in a patient suffering from a plaque-forming disease, comprising the step of administering to the patient a pharmaceutical composition comprising filamentous bacteriophage; and a pharmaceutically acceptable carrier, wherein the composition comprises less than 1×10⁻¹⁰ endotoxin units per filamentous bacteriophage.

18. The method of claim 17, wherein the filamentous bacteriophage is selected from wild-type filamentous bacteriophage or filamentous bacteriophage which does not display an antibody or a non-filamentous bacteriophage antigen on its surface.

19. The method of claim 17, wherein the plaque-forming disease is selected from Alzheimer's disease, SAA amyloidosis, hereditary Icelandic Syndrome, senility, multiple myeloma, Kuru, Creutzfeldt-Jakob Disease (CJD), Gerstmann-Straussler-Scheinker disease (GSS), fatal familial insomnia (FFI), scrapie, bovine spongiform encephalitis (BSE), Parkinson's Disease, Amyotrophic lateral sclerosis/parkinsonism-dementia complex, Argyrophilic grain dementia, Corticobasal degeneration, Dementia pugilistica, diffuse neurofibrillary tangles with calcification, Down's syndrome, Frontotemporal dementia with parkinsonism linked to chromosome 17, Hallervorden-Spatz disease, Myotonic dystrophy, Niemann-Pick disease type C, Non-Guamanian motor neuron disease with neurofibrillary tangles, Pick's disease, Postencephalitic parkinsonism, Progressive subcortical gliosis, Progressive supranuclear palsy, Subacute sclerosing panencephalitis, and Tangle only dementia.

20. The method of claim 19, wherein the plaque-forming disease is selected from early onset Alzheimer's disease, late onset Alzheimer's disease or pre-symptomatic Alzheimer's disease.
